(19) United States
(12) Patent Application Publication
(10) Pub. No.: US 2005/0058990 A1
(43) Pub. Date: Mar. 17, 2005

(54) Biochip devices for ion transport measurement, methods of manufacture, and methods of use

(76) Inventors: Antonio Guia, San Diego, CA (US); Jia Xu, San Diego, CA (US); Lei Wu, San Diego, CA (US); Khachonesin Sithiphong, San Diego, CA (US); Maria Spassova, Bala Cynwyd, PA (US); Huimin Tao, San Diego, CA (US); George Walker, San Diego, CA (US); Mingxian Huang, San Diego, CA (US); Guoliang Tao, San Diego, CA (US); Steven Saya, San Diego, CA (US); Glenn Walker, San Marcos, CA (US); Zoya Zozulya, La Mesa, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/858,339

(22) Filed: Jun. 1, 2004

Related U.S. Application Data
(63) Continuation-in-part of application No. 10/760,866, filed on Jan. 20, 2004.
Application No. 10/760,866 is a continuation-in-part of application No. 10/428,565, filed on May 2, 2003, now abandoned.
(63) Continuation-in-part of application No. 10/642,014, filed on Aug. 16, 2003.
Application No. 10/642,014 is a continuation-in-part of application No. 10/351,019, filed on Jan. 23, 2003, now abandoned.
(63) Continuation-in-part of application No. 10/104,300, filed on Mar. 22, 2002.
(60) Provisional application No. 60/380,007, filed on May 4, 2002.
(60) Provisional application No. 60/351,849, filed on Jan. 24, 2002.
(60) Provisional application No. 60/311,327, filed on Aug. 10, 2001.
(60) Provisional application No. 60/278,308, filed on Mar. 24, 2001.
(60) Provisional application No. 60/474,508, filed on May 31, 2003.

(51) Int. Cl. G01N 15/06; C12Q 1/70

(52) U.S. Cl. 435/5; 422/68.1

(57) ABSTRACT

The present invention provides biochips for ion transport measurement, ion transport measuring devices that comprise biochips, and methods of using ion transport measuring devices and biochips that allow for the direct analysis of ion transport functions or properties. The present invention provides biochips, devices, apparatuses, and methods that allow for automated detection of ion transport functions or properties. The present invention also provides methods of making biochips and devices for ion transport measurement that reduce the cost and increase the efficiency of manufacture, as well as improve the performance of the biochips and devices. These biochips and devices are particularly appropriate for automating the detection of ion transport functions or properties, particularly for screening purposes.

This application is a continuation-in-part of U.S. patent application Ser. No. 10/760,866 (pending), filed Jan. 20, 2004, which is a continuation-in-part of U.S. patent application Ser. No. 10/428,565, filed May 2, 2003 (abandoned), which claims benefit of priority to U.S. patent application No. 60/380,007, filed May 4, 2002 (expired); a continuation-in-part of U.S. patent application Ser. No. 10/642,014, filed Aug. 16, 2003 (pending), which claims priority to U.S. patent application Ser. No. 10/351,019, filed Jan. 23, 2003 (abandoned), which claims priority to U.S. patent application No. 60/351,849 filed Jan. 24, 2002 (expired); and a continuation-in-part of U.S. patent application Ser. No. 10/104,300, filed Mar. 22, 2002 (pending), which claims priority to U.S. patent application No. 60/311,327 filed Aug. 10, 2001 (expired) and to U.S. patent application No. 60/278,308 filed Mar. 24, 2001 (expired). This application also claims priority to U.S. patent application No. 60/474,508 filed May 31, 2003. Each and every patent or patent application referred to in this paragraph is hereby incorporated by reference herein in its entirety.

### TECHNICAL FIELD

The present invention relates generally to the field of ion transport detection (“patch clamp”) systems and methods, particularly those that relate to the use of biochip technologies.

### BACKGROUND

Ion transports are channels, transporters, pore forming proteins, or other entities that are located within cellular membranes and regulate the flow of ions across the membrane. Ion transports participate in diverse processes, such as generating and timing of action potentials, synaptic transmission, secretion of hormones, contraction of muscles etc. Ion transports are popular candidates for drug discovery, and many known drugs exert their effects via modulation of ion transport functions or properties. For example, antiepileptic compounds such as phenytoin and lamotrigine which block voltage dependent sodium ion transports in the brain, anti-hypertension drugs such as nifedipine and diltiazem which block voltage dependent calcium ion transports in smooth muscle cells, and stimulators of insulin release such as glibenclamide and tolbutamine which block an ATP regulated potassium ion transport in the pancreas.

One popular method of measuring an ion transport function or property is the patch-clamp method, which was first reported by Neher, Sakmann and Steinback (Pflueger Arch. 375:219-278 (1978)). This first report of the patch clamp method relied on pressing a glass pipette containing acetylcholine (Ach) against the surface of a muscle cell membrane, where discrete jumps in electrical current were attributable to the opening and closing of Ach-activated ion transports.

The method was refined by fire polishing the glass pipettes and applying gentle suction to the interior of the pipette when contact was made with the surface of the cell. Seals of very high resistance (between about 1 and about 100 giga ohms) could be obtained. This advancement allowed the patch clamp method to be suitable over voltage ranges which ion transport studies can routinely be made.

A variety of patch clamp methods have been developed, such as whole cell, vesicle, outside-out and inside-out patches (Liem et al., Neurosurgery 36:382-392 (1995)). Additional methods include whole cell patch clamp recordings, pressure patch clamp methods, cell free ion transport recording, perfusion patch pipettes, concentration patch clamp methods, perforated patch clamp methods, loose patch voltage clamp methods, patch clamp recording and patch clamp methods in tissue samples such as muscle or brain (Boulton et al, Patch-Clamp Applications and Protocols, Neuromethods V. 26 (1995), Humana Press, New Jersey).

These and later methods relied upon interrogating one sample at a time using large laboratory apparatuses that require a high degree of operator skill and time. Attempts have been made to automate patch clamp methods, but these have met with little success. Alternatives to patch clamp methods have been developed using fluorescent probes, such as cumarin-lipids (cu-lipids) and oxonol fluorescent dyes (Tsien et al., U.S. Pat. No. 6,107,066, issued August 2000). These methods rely upon change in polarity of membranes and the resulting motion of oxonol molecules across the membrane. This motion allows for the detection of changes in fluorescence resonance energy transfer (FRET) between cu-lipids and oxonol molecules. Unfortunately, these methods do not measure ion transport directly but measure the change of indirect parameters as a result of ionic flux. For example, the characteristics of the lipid used in the cu-lipid can alter the biological and physical characteristics of the membrane, such as fluidity and polarizability.

Thus, what is needed is a simple device and method to measure ion transport directly. Preferably, these devices would utilize patch clamp detection methods because these types of methods represent a gold standard in this field of study. The present invention provides these devices and methods particularly miniaturized devices and automated methods for the screening of chemicals or other moieties for their ability to modulate ion transport functions or properties.

### BRIEF SUMMARY OF THE INVENTION

The present invention recognizes that the determination of one or more ion transport functions or properties using direct detection methods, such as patch-clamp, whole cell recording, or single channel recording, are preferable to methods that utilize indirect detection methods, such as fluorescence-based detection systems.

The present invention provides biochips for ion transport measurement, ion transport measuring devices that comprise biochips, and methods of using ion transport measuring devices and biochips that allow for the direct analysis of ion transport functions or properties. The present invention provides biochips, devices, apparatuses, and methods that allow for automated detection of ion transport functions or properties. The present invention also provides methods of making biochips and devices for ion transport measurement that reduce the cost and increase the efficiency of manufacture, as well as improve the performance of the biochips and devices. These biochips and devices are particularly appropriate for automating the detection of ion transport functions or properties, particularly for screening purposes.

A first aspect of the present invention is a biochip device for ion transport measurement. A biochip device comprises an upper chamber piece that comprises one or more upper chambers and a biochip that comprises at least one ion transport measuring means. In one preferred embodiment of this aspect of the present invention, a biochip device is part of an apparatus that also comprises at least one conduit that that can be positioned to engage the one or more upper chambers, where the conduit comprises an electrode or can provide an electrolyte bridge to an electrode.

A second aspect of the present invention is a biochip device having one or more flow-through lower chambers. The device comprises an upper chamber piece that comprises one or more upper chambers, a biochip that comprises at least one ion transport measuring means, and at least one lower chamber base piece that comprises one or more lower chambers and at least two conduits that connect with at least one of the one or more lower chambers.

A third aspect of the invention is biochip-based ion transport measurement devices that are adapted for microscope stages. The devices comprise an upper chamber piece that comprises one or more upper chambers, a biochip that comprises at least one ion transport measuring means, and at least one lower chamber base piece, in which the bottom surface of the lower chamber base piece is transparent. Preferably, the device also includes a baseplate adapted to a microscope stage into which a lower chamber base piece can fit.

A fourth aspect of the invention is methods of making an upper chamber piece for a biochip device for ion transport measurement. In one preferred embodiment of this aspect of the present invention, an upper chamber piece can be molded as two pieces, an upper well portion piece and a well hole portion piece. Preferably, a well hole portion piece comprises at least one groove into which at least one electrode can be inserted. After insertion of the electrode, the upper well portion piece and the well hole portion piece are attached to form an upper chamber piece. In another embodiment of this aspect, an upper chamber piece can be molded as a single piece, where an electrode, such as a wire electrode, can be positioned in a mold and then the upper chamber piece can be molded around it. In yet another preferred embodiment of this aspect, an upper chamber piece can be molded as a single piece without an electrode.

A fifth aspect of the invention is methods for making chips comprising ion transport measuring holes. An ion transport measuring hole can be fabricated by laser drilling one or more counterbores, and then laser drilling a through-hole through the one or more counterbores.

A sixth aspect of the invention is an ion transport measuring device that comprises an inverted chip comprising ion transport measuring holes. A chip used in inverted orientation can comprise one or more ion transport measuring holes that are fabricated by laser drilling of one or more counterbores and a through-hole through the one or more counterbores.

A seventh aspect of the invention is methods of treating ion transport measuring chips to enhance their sealing properties. In one aspect of the present invention, the chip or substrate comprising an ion transport measuring means is modified to become more electronegative, more smooth, or more electronegative and more smooth. In some aspects of the present invention, the chip or substrate comprising the ion transport measuring means is modified chemically, such as with acids, bases, or a combination thereof. Treatment of chips of the present invention with chemical solution can be performed using treatment racks that fit into vessels that hold the chemical solutions and can hold multiple glass chips while allowing access of the chemical solutions to the chip surfaces.

An eighth aspect of the invention is a method to measure surface energy on a surface, such as the surface of a chemically-treated ion transport measurement biochip. The surface energy measurement can be used to evaluate the hydrophilicity of a biochip biochip of the present invention that has been chemically treated to improve its electrical sealing properties, such as, for example, at chip that has been treated with base. The method can also be used for any surface characterization purpose where a measurement of surface energy or hydrophilicity is desired.

A ninth aspect of the invention is the substrates, biochips, devices, apparatuses, and/or cartridges comprising ion transport measuring means with enhanced electric seal properties. In preferred embodiments, at least a portion of at least one chip that comprises at least one ion transport measuring means has been modified to become more electronegative. In preferred embodiments, at least a portion of at least one chip that comprises at least one ion transport measuring means has been treated with at least one base, at least one acid, or both.

A tenth aspect of the present invention is a method for storing the substrates, biochips, cartridges, apparatuses, and/or devices comprising ion transport measuring means with enhanced electrical seal properties.

An eleventh aspect of the present invention is a method for shipping the substrates, biochips, cartridges, apparatuses, and/or devices comprising ion transport measuring means with enhanced electrical seal properties.

A twelfth aspect of the invention is methods for assembling devices and cartridges of the present invention. The methods include attaching an upper chamber piece to a biochip that comprises at least one ion transport measuring means using a UV adhesive. Preferably, the chip has been chemically treated to enhance its electrical sealing properties. During UV activation of the adhesive, at least a portion of the biochip is masked to prevent UV irradiation of ion transport measuring means on the chip.

A thirteenth aspect of the present invention is a method of producing biochips comprising ion transport measuring means by fabricating the biochips as detachable units of a large sheet. Ion transport measuring holes can be made by wet etching and laser drilling appropriate substrates, and the sheet can be scored with a laser such that portions of the sheet having a desired number of ion transport measuring holes can be separated along the score lines. In some embodiments, upper chamber pieces are attached to the substrate sheet after the fabrication of holes and before separation of sections of the sheet. In this case, the detachable units that are separated to produce devices comprise cartridges having upper chambers attached to an ion transport measuring chip.

A fourteenth aspect of the invention is a method of producing high density ion transport measuring chips. The ion transport measuring chips preferably have more than 16 ion transport measuring holes, and wells can be fabricated in a chip using wet etching, followed by laser drilling of ion transport measuring holes through the bottoms of the wells.

A fifteenth aspect of the invention is a biochip device for ion transport measurement comprising fluidic channel upper and lower chambers. The fluidic channels have apertures that are aligned with ion transport measuring holes on the chip. The fluidic channels can be connected to sources for generating or promoting fluid flow, such as pumps, pressure sources, and valves. The fluidic channels preferably provide electrolyte bridges to one or more electrodes that can be used in ion transport measurement.

A sixteenth aspect of the present invention is methods of preparing cells for ion transport measurement. The methods include the use of filters that can allow the passage of single cells through their pores and monitoring of cell health parameters important for electrophysiological measurements.

A seventeenth aspect of the present invention is a logic and program that uses a pressure control profile to direct an ion transport measurement apparatus to achieve and maintain a high-resistance electrical seal. The logic can follow decision pathways based on information from electrical measurements made by ion transport measuring electrodes in a feedback system.

### BRIEF DESCRIPTION OF THE DRAWINGS

FIG. 1 depicts four views of one example of an upper chamber piece of the present invention: A) top view; B) bottom view; C) side-on cross-sectional view; and D) end-on cross-sectional view.

FIG. 2 depicts a cross-sectional view of a single ion transport measuring unit of one example of an ion transport measuring device of the present invention. Figure is not necessarily to scale.

FIG. 3 provides photographs of a lower chamber piece of the present invention that is adapted to fit a microscope stage and has flow-through lower chambers. (A) view of a plastic lower chamber base piece with connectors for inflow and outflow tubes, B) a zoomed-in view of the lower chamber base piece showing inflow and outflow tubes C) the lower chamber piece installed in a base plate.

FIG. 4 provides photographs of one design of a base plate for adapting a biochip device to a microscope stage. (A) Top view and (B) bottom view of a base plate cut from aluminum stock. The holes (401) are threaded except for the four holes closest to the corners of the square-cut carve-out. The four unthreaded holes (402) are sized to accept a press-in 1 mm socket connector.

FIG. 5 depicts one device of the present invention having a lower chamber base piece fitted to a baseplate (54) by means of a clamp (53) which also attaches the upper chamber piece (51) to the lower chamber base piece (not visible). The clamp also comprises wire electrodes (55) that extend into upper wells. Electrode connectors (52) have wires extending into the fluidics of each lower chamber below.

FIG. 6 depicts a lower chamber piece of the present invention in the form of a gasket having multiple holes (601) that form the walls of lower chambers in an assembled device. In this design, the holes are formed by O-ring structures (602).

FIG. 7 provides photographs of a clamp part (A) upside down and (B) viewed from the top fitted over a cartridge.

FIG. 8 provides photographs of a cartridge device of the present invention (black item) shown in relation to the rest of the parts of a device adapted for a microscope (A) and after assembly into a baseplate (B).

FIG. 9 depicts an upper chamber piece of the present invention that is made from an upper well portion piece (91) and a well-hole portion piece (92). (A) the upper well portion piece (91) is shown above the well-hole portion piece (92). (B) the upper well portion piece (91) is shown fitted on the well-hole portion piece to form wells (93), with the groove (94) where an electrode can be inserted visible along the back of the wells (93).

FIG. 10 is a graph that illustrates that a decreasing hole depth (x-axis) and widening the exit hole (as for “K-configuration” chips) decreases Re (y-axis). On the left side (“K-configuration” chips): black circles, chips having 2.5 micron diameter holes with 6 micron entrance holes; black squares, chips having 2 micron diameter holes with 5 micron entrance holes; black double triangles, chips having 1.8 micron diameter holes with 4 and 6 micron entrance holes; and X's, chips having 1.5 micron diameter holes with 6 micron entrance holes. On the right side (“S-configuration chips) black triangles, chips having 2.5 micron diameter holes with 10 micron entrance holes; black squares, chips having 2 micron diameter holes with 9 micron entrance holes; open triangles, chips having 1.8 micron diameter holes with 7 micron entrance holes; and black diamonds, chips having 1.5 micron diameter holes with 8 micron entrance holes.

FIG. 11 is a graph illustrating that thinner chips (for example “K-configuration” chips of the present invention) have a lower Ra (“improved Ra”) than those with greater hole depth. Ra also decreases as hole diameter increases, however at a cost of lower Rm. Increased Rm (“improved Rm”) is found with increased hole depth.

FIG. 12 gives depictions of a laser drilled chip (123) having a first counterbore (126) and a second counterbore (127) and a through-hole (128). In A) the direction of laser drilling of the counterbores (126 and 127) and through-hole (128) is shown by the arrow. In B), the chip is used in inverted orientation with a cell (129) sealed to the hole (128) that connects the upper chamber (121) with the lower chamber (125) having walls formed by a gasket (124). Figure is not necessarily to scale.

FIG. 13 depicts treatment fixtures for chemically treating chips and devices. (A) shows a single layer treatment fixture that can fit into a glass jar containing acid, base, or other chemical solutions. (B) shows the stacked fixture.

FIG. 14 shows one design of a shipping fixture for cartridges of the present invention. In A), a blister pack having a plastic frame (141) and openings (142) for sealing cartridges (143) is viewed from the bottom. In B), the blister pack is viewed from the top side of the sealed-in cartridge (143).

FIG. 15 depicts a glass chip (151) with multiple ion transport holes (152) that can be attached to a multichamber upper chamber piece to form a multiunit sheet (154). The multiunit sheet (154) comprising upper chambers and a chip (151) has mark lines or perforations in the chip (153) where the sheet can be separated into sections. Cartridges with a smaller number of units (155) can be separated from the larger multiunit sheet (154). Not to scale.

FIG. 16 depicts one example of a high density array chip (161) of the present invention. The wells (162) of the chip can be made by wet etching followed by laser drilling through holes through the bottoms of the wells (162).

FIG. 17 shows an example of a high density array having upper chambers (171) that can be formed by a well plate (172) attached to the chip (173). Wells (174) in the chip (173) having laser drilled through-holes can be oriented in inverted orientation (top alternative) or standard orientation (bottom alternative).

FIG. 18 depicts the general format for pressure bonding, in which a chip (183) comprising a hole (182) is attached to an upper chamber piece (181) using a gasket (184) to form a seal between the upper chamber piece (181) and chip (183) when pressure (arrow) is applied. In this highly schematized depiction, a lower chamber piece (185) is also attached to the chip (183) using a second gasket (186) to form a seal between the lower chamber piece (185) and chip (183) when pressure (arrow) is applied.

FIG. 19 depicts a schematic view of one design a planar patch clamping chip (193) having an upper fluid channel (191) for extracellular solution (ES) and a lower fluidic channel (195) for intracellular solutions (IS 1, IS2). The upper and lower channels are interfaced at a point where the recording aperture (192) of the planar electrode resides. Separate fluidic pumps (P) drive the flow of fluids through the two (upper and lower) fluidic channels. Recording (196) and reference electrodes (197) external to the fluidic patch clamp chip are connected via an electrolyte solution bridge to the upper (191) and lower (195) fluidic channels. A pressure source such as a pump with pressure controller that can generate both positive and negative pressures is linked to the lower fluidic channels. A multi-way valve (194) is used to connect the lower fluidic channel (195) to different solution reservoirs (IS 1, IS2, etc), and a multi-way valve (198) is used to connect the upper fluidic channel (191) to cell reservoirs, compound plate (CP), wash buffers and other solutions. (Not to scale).

FIG. 20 provides graphs of the success rate of a test of patch clamp seals using cartridges of the present invention having chemically treated chips. A) gives the success duration of seals on 52 chips. B) plots the accumulative success rate of cells on 53 chips (achieved gigaseals and gave Ra<15MOhm and Rm>200MOhm throughout 15 min recording period).

FIG. 21 provides graphs of results of tests performed on 52 chips. A) gives Re values of the chips. B) gives break-in pressures during the quality control test.

FIG. 22 provides graphs of Rm (membrane resistance) and Ra (access resistance) at the beginning and at end of tests using devices of the present invention. A) shows Rm after break-in (wide diagonals slanting upward) and at the end of the test (narrow diagonals slanting downward). B) shows Ra after break-in (wide diagonals slanting upward) and at the end of the test (narrow diagonals slanting downward).

FIG. 23 provides typical patch clamp recordings immediately after break-in using a device of the present invention. A) uncorrected whole-cell recording, B) corrected whole cell recording, C) plot of corrected and uncorrected recording taken during the interval denoted by the arrowheads in A) and B).

FIG. 24 provides typical patch clamp recordings fifteen minutes after break-in using a device of the present invention. A) uncorrected whole cell recording, B) corrected whole cell recording, C) plot of corrected and uncorrected recording taken during the interval denoted by the arrowheads in A) and B).

FIG. 25 plots the Rm and Ra values for patch clamps of the experiment shown in FIGS. 23 and 24 beginning at break-in and continuing over a 15-minute period.

FIG. 26 is a flowchart of an overview of the pressure control profile program.

FIG. 27 is a flowchart of part 1 of Procedure Landing of the pressure control profile program.

FIG. 28 shows a flowchart of part 2 of Procedure Landing of the pressure control profile program.

FIG. 29 shows a flowchart of part 3 of Procedure Landing of the pressure control profile program.

FIG. 30 shows a flowchart of part 1 of Procedure FormSeal of the pressure control profile program.

FIG. 31 shows a flowchart of part 2 of Procedure FormSeal of the pressure control profile program.

FIG. 32 shows a flowchart of part 3 of Procedure FormSeal of the pressure control profile program.

FIG. 33 shows a flowchart of part 4 of Procedure FormSeal of the pressure control profile program.

FIG. 34 shows a flowchart of part 5 of Procedure FormSeal of the pressure control profile program.

FIG. 35 shows a flowchart of part 1 of Procedure BreakIn of the pressure control profile program.

FIG. 36 shows a flowchart of part 2 of Procedure BreakIn of the pressure control profile program.

FIG. 37 shows a flowchart of part 3 of Procedure BreakIn of the pressure control profile program.

FIG. 38 shows a flowchart of part 4 of Procedure BreakIn of the pressure control profile program.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Terms of orientation such as “up” and “down”, “top” and “bottom”, “upper” or “lower” and the like refer to orientation of parts during use of a device. Where a term is provided in the singular, the inventors also contemplate the plural of that term. Where there are discrepancies in terms and definitions used in references that are incorporated by reference, the terms used in this application shall have the definitions given herein. As employed throughout the disclosure, the following terms, unless otherwise indicated, shall be understood to have the following meanings:

“Ion transport measurement” is the process of detecting and measuring the movement of charge and/or conducting ions across a membrane (such as a biological membrane), or from the inside to the outside of a particle or vice versa. In most applications, particles will be cells, organelles, vesicles, biological membrane fragments, artificial membranes, bilayers or micelles. In general, ion transport measurement involves achieving a high resistance electrical seal of a membrane or particle with a surface that has an aperture, and positioning electrodes on either side of the membrane or particle to measure the current and/or voltage across the portion of the membrane sealed over the aperture, or “clamping” voltage across the membrane and measuring current applied to an electrode to maintain that voltage. However, ion transport measurement does not require that a particle or membrane be sealed to an aperture if other means can provide electrode contact on both sides of a membrane. For example, a particle can be impaled with a needle electrode and a second electrode can be provided in contact with the solution outside the particle to complete a circuit for ion transport measurement. Several techniques collectively known as “patch clamping” can be included as “ion transport measurement”.

An “ion transport measuring means” refers to a structure that can be used to measure at least one ion transport function, property, or a change in ion channel function, property in response to various chemical, biochemical or electrical stimuli. Typically, an ion transport measuring means is a structure with an opening that a particle can seal against, but this need not be the case. For example, needles as well as holes, apertures, capillaries, and other detection structures of the present invention can be used as ion transport measuring means. An ion transport measuring means is preferably positioned on or within a biochip or a chamber. Where an ion transport measuring means refers to a hole or aperture, the use of the terms “ion transport measuring means” “hole” or “aperture” are also meant to encompass the perimeter of the hole or aperture that is in fact a part of the chip or substrate (or coating) surface (or surface of another structure, for example, a channel) and can also include the surfaces that surround the interior space of the hole that is also the chip or substrate (or coating) material or material of another structure that comprises the hole or aperture.

A “hole” is an aperture that extends through a chip. Descriptions of holes found herein are also meant to encompass the perimeter of the hole that is in fact a part of the chip or substrate (or coating) surface, and can also include the surfaces that surround the interior space of the hole that is also the chip or substrate (or coating) material. Thus, in the present invention, where particles are described as being positioned on, at, near, against, or in a hole, or adhering or fixed to a hole, it is intended to mean that a particle contacts the entire perimeter of a hole, such that at least a portion of the surface of the particle lies across the opening of the hole, or in some cases, descends to some degree into the opening of the whole, contacting the surfaces that surround the interior space of the hole.

A “patch clamp detection structure” refers to a structure that is on or within a biochip or a chamber that is capable of measuring at least one ion transport function or property via patch clamp methods.

A “chip” is a solid substrate on which one or more processes such as physical, chemical, biochemical, biological or biophysical processes can be carried out. Such processes can be assays, including biochemical, cellular, and chemical assays; ion transport or ion channel function or activity determinations, separations, including separations mediated by electrical, magnetic, physical, and chemical (including biochemical) forces or interactions; chemical reactions, enzymatic reactions, and binding interactions, including captures. The micro structures or micro-scale structures such as for example, channels and wells, electrode elements, or electromagnetic elements, may be incorporated into or fabricated on the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips of the present invention can vary considerably, for example, from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include wells fabricated on the surfaces.

A “biochip” is a chip that is useful for a biochemical, biological or biophysical process. In this regard, a biochip is preferably biocompatible, in that it does not negatively affect cells or cell membranes.

A “chamber” is a structure that comprises or engages a chip and that is capable of containing a fluid sample. The chamber may have various dimensions and its volume may vary between 0.001 microliter and 50 milliliter. In devices of the present invention, an “upper chamber” is a chamber that is above a biochip, such as a biochip that comprises one or more ion transport measuring means. In the devices of the present invention, a chip that comprises one or more ion transport measuring means can separate one or more upper chambers from one or more lower chambers. During use of a device, an upper chamber can contain measuring solutions and particles or membranes. An upper chamber can optionally comprise one or more electrodes. In devices of the present invention, a “lower chamber” is a chamber that is below a biochip. During use of a device, a lower chamber can contain measuring solutions and particles or membranes. A lower chamber can optionally comprise one or more electrodes.

A lower chamber “has access to” or “accesses” an upper chamber via (or through) a hole in a chip when the chip separates or is between the upper and lower chambers and a hole in the chip provides fluid communication between the referenced lower chamber and the referenced upper chamber. An upper chamber “has access to” or “accesses” a lower chamber via (or through) a hole in a chip when the chip separates or is between the upper and lower chambers and a hole in the chip provides fluid communication between the referenced upper chamber and the referenced lower chamber. Similarly an upper chamber can be “connected to” a lower chamber (or vice versa) via a hole in a chip when the hole in the chip provides fluid communication between the referenced upper chamber and the referenced lower chamber.

A “lower chamber piece” is a part of a device for ion transport measurement that forms at least a portion of one or more lower chambers of the device. A lower chamber piece preferably comprises at least a portion of one or more walls of one or more lower chambers, and can optionally comprise at least a portion of a bottom surface of one or more lower chambers, and can optionally comprise one or more conduits that lead to one or more lower chambers, or one or more electrodes.

A “lower chamber base piece” or “base piece” is a part of a device for ion transport measurement that forms the bottom surface of one or more lower chambers of the device. A lower chamber base piece can also optionally comprise one or more walls of one or more lower chambers, one or more conduits that lead to one or more lower chambers, or one or more electrodes.

As used herein, a “platform” is a surface on which a device of the present invention can be positioned. A platform can comprises the bottom surface of one or more lower chambers of a device.

An “upper chamber piece” is a part of a device for ion transport measurement that forms at least a portion of one or more upper chambers of the device. An upper chamber piece can comprise one or more walls of one or more upper chambers, and can optionally comprise one or more conduits that lead to an upper chamber, and one or more electrodes.

An “upper chamber portion piece” is a part of a device for ion transport measurement that forms a portion of one or more upper chambers of the device. An upper chamber portion piece can comprise at least a portion of one or more walls of one or more upper chambers, and can optionally comprise one or more conduits that lead to an upper chamber, or one or more electrodes.

A “well” is a depression in a substrate or other structure. For example, in devices of the present invention, upper chambers can be wells formed in an upper chamber piece. The upper opening of a well can be of any shape and can be of an irregular conformation. The walls of a well can extend upward from the lower surface of a well at any angle or in any way. The walls can be of any shape and can be of an irregular conformation, that is, they may extend upward in a sigmoidal or otherwise curved or multi-angled fashion.

A “well hole” is a hole in the bottom of a well. A well hole can be a well-within-a well, having its own well shape with an opening at the bottom.

A “well hole piece” is a part of a device for ion transport measurement that comprises one or more well holes of the wells of the device.

When wells or chambers (including fluidic channel chambers) are “in register with” ion transport measuring means of a chip, there is a one-to-one correspondence of each of the referenced wells or chambers to each of the referenced ion transport measuring means, and an ion transport measuring means is positioned so that it is exposed to the interior of the well or chamber it is in register with, such that ion transport measurement can be performed using the chamber as a compartment for measuring current or voltage through or across the ion transport measuring means.

A “port” is an opening in a wall or housing of a chamber through which a fluid sample or solution can enter or exit the chamber. A port can be of any dimensions, but preferably is of a shape and size that allows a sample or solution to be dispensed into a chamber by means of a pipette, syringe, or conduit, or other means of dispensing a sample.

A “conduit” is a means for fluid to be transported into or out of a device, apparatus, or system for ion transport measurement of the present invention or from one area to another area of a device, apparatus, or system of the present invention. In some aspects, a conduit can engage a port in the housing or wall of a chamber. In some aspects, a part of a device, such as, for example, an upper chamber piece or a lower chamber piece can comprise conduits in the form of tunnels that pass through the upper chamber piece and connect, for example, one area or compartment with another area or compartment. A conduit can be drilled or molded into a chip, chamber, housing, or chamber piece, or a conduit can comprise any material that permits the passage of a fluid through it, and can be attached to any part of a device. In one preferred aspect of the present invention, a conduit extends through at least a portion of a device, such as a wall of a chamber, or an upper chamber piece or lower chamber piece, and connects the interior space of a chamber with the outside of a chamber, where it can optionally connect to another conduit, such as tubing. Some preferred conduits can be tubing, such as, for example, rubber, teflon, or tygon tubing. A conduit can be of any dimensions, but preferably ranges from 10 microns to 5 millimeters in internal diameter.

A “device for ion transport measurement” or an “ion transport measuring device” is a device that comprises at least one chip that comprises one or more ion transport measuring means, at least a portion of at least one upper chamber, and, preferably, at least a portion of at least one lower chamber. A device for ion transport measurement preferably comprises one or more electrodes, and can optionally comprise conduits, particle positioning means, or application-specific integrated circuits (ASICs).

A “cartridge for ion transport measurement” comprises an upper chamber piece and at least one biochip comprising one or more ion transport measuring means attached to the upper chamber piece, such that the one or more ion transport measuring means are in register with the upper chambers of the upper chamber piece.

An “ion transport measuring unit” is a portion of a device that comprises at least a portion of a chip having a single ion transport measuring means and a single upper chamber, where the ion transport measuring means is in register with the upper chamber. An ion transport measuring unit can further comprise at least a portion of a lower chamber that is in register with the ion transport measuring means an upper chamber.

A “measuring solution” is an aqueous solution containing electrolytes, with pH, osmolarity, and other physical-chemical traits that are compatible with conducting function of the ion transports to be measured.

An “intracellular solution” is a measuring solution used in the upper or lower chamber that is compatible with the electrolyte composition and physical-chemical traits of the intracellular content of a living cell.

An “extracellular solution” is a measuring solution used in the upper or lower chamber that is compatible with the electrolyte composition and physical-chemical traits of the extracellular content of a living cell.

To be “in electrical contact with” means one component is able to receive and conduct electrical signals (for example, voltage, current, or change of voltage or current) from another component.

An “ion transport” can be any protein or non-protein moiety that modulates, regulates or allows transfer of ions across a membrane, such as a biological membrane or an artificial membrane. Ion transport include but are not limited to ion channels, proteins allowing transport of ions by active transport, proteins allowing transport of ions by passive transport, toxins such as from insects, viral proteins or the like. Viral proteins, such as the M2 protein of influenza virus can form an ion channel on cell surfaces.

A “particle” refers to an organic or inorganic particulate that is suspendable in a solution and can be manipulated by a particle positioning means. A particle can include a cell, such as a prokaryotic or eukaryotic cell, or can be a cell fragment, such as a vesicle or a microsome that can be made using methods known in the art. A particle can also include artificial membrane preparations that can be made using methods known in the art. Preferred artificial membrane preparations are lipid bilayers, but that need not be the case. A particle in the present invention can also be a lipid film, such as a black-lipid film (see, Houslay and Stanley, Dynamics of Biological Membranes, Influence on Synthesis, Structure and Function, John Wiley & Sons, New York (1982)). In the case of a lipid film, a lipid film can be provided over a hole, such as a hole or capillary of the present invention using methods known in the art (see, Houslay and Stanley, Dynamics of Biological Membranes, Influence on Synthesis, Structure and Function, John Wiley & Sons, New York (1982)). A particle preferably includes or is suspected of including at least one ion transport or an ion transport of interest. Particles that do not include an ion transport or an ion transport of interest can be made to include such ion transport using methods known in the art, such as by fusion of particles or insertion of ion transports into such particles such as by detergents, detergent removal, detergent dilution, sonication or detergent catalyzed incorporation (see, Houslay and Stanley, Dynamics of Biological Membranes, Influence on Synthesis, Structure and Function, John Wiley & Sons, New York (1982)). A microparticle, such as a bead, such as a latex bead or magnetic bead, can be attached to a particle, such that the particle can be manipulated by a particle positioning means.

A “cell” refers to a viable or non-viable prokaryotic or eukaryotic cell. A eukaryotic cell can be any eukaryotic cell from any source, such as obtained from a subject, human or non-human, fetal or non-fetal, child or adult, such as from a tissue or fluid, including blood, which are obtainable through appropriate sample collection methods, such as biopsy, blood collection or otherwise. Eukaryotic cells can be provided as is in a sample or can be cell lines that are cultivated in vitro. Differences in cell types also include cellular origin, distinct surface markers, sizes, morphologies and other physical and biological properties.

A “cell fragment” refers to a portion of a cell, such as cell organelles, including but not limited to nuclei, endoplasmic reticulum, mitochondria or golgi apparatus. Cell fragments can include vesicles, such as inside out or outside out vesicles or mixtures thereof. Preparations that include cell fragments can be made using methods known in the art.

A “population of cells” refers to a sample that includes more than one cell or more than one type of cell. For example, a sample of blood from a subject is a population of white cells and red cells. A population of cells can also include a sample including a plurality of substantially homogeneous cells, such as obtained through cell culture methods for a continuous cell lines.

A “population of cell fragments” refers to a sample that includes more than one cell fragment or more than one type of cell fragments. For example, a population of cell fragments can include mitochondria, nuclei, microsomes and portions of golgi apparatus that can be formed upon cell lysis.

A “microparticle” is a structure of any shape and of any composition that is manipulatable by desired physical force(s). The microparticles used in the methods could have a dimension from about 0.01 micron to about ten centimeters. Preferably, the microparticles used in the methods have a dimension from about 0.1 micron to about several hundred microns. Such particles or microparticles can be comprised of any suitable material, such as glass or ceramics, and/or one or more polymers, such as, for example, nylon, polytetrafluoroethylene (TEFLON™), polystyrene, polyacrylamide, sepaharose, agarose, cellulose, cellulose derivatives, or dextran, and/or can comprise metals. Examples of microparticles include, but are not limited to, plastic particles, ceramic particles, carbon particles, polystyrene microbeads, glass beads, magnetic beads, hollow glass spheres, metal particles, particles of complex compositions, microfabricated free-standing microstructures, etc. The examples of microfabricated free-standing microstructures may include those described in “Design of asynchronous dielectric micromotors” by Hagedorn et al., in Journal of Electrostatics, Volume: 33, Pages 159-185 (1994). Particles of complex compositions refer to the particles that comprise or consists of multiple compositional elements, for example, a metallic sphere covered with a thin layer of non-conducting polymer film.

“A preparation of microparticles” is a composition that comprises microparticles of one or more types and can optionally include at least one other compound, molecule, structure, solution, reagent, particle, or chemical entity. For example, a preparation of microparticles can be a suspension of microparticles in a buffer, and can optionally include specific binding members, enzymes, inert particles, surfactants, ligands, detergents, etc.

“Coupled” means bound. For example, a moiety can be coupled to a microparticle by specific or nonspecific binding. As disclosed herein, the binding can be covalent or noncovalent, reversible or irreversible.

“Micro-scale structures” are structures integral to or attached on a chip, wafer, or chamber that have characteristic dimensions of scale for use in microfluidic applications ranging from about 0.1 micron to about 20 mm. Example of micro-scale structures that can be on chips of the present invention are wells, channels, scaffolds, electrodes, electromagnetic units, or microfabricated pumps or valves.

A “particle positioning means” refers to a means that is capable of manipulating the position of a particle relative to the X-Y coordinates or X-Y-Z coordinates of a biochip. Positions in the X-Y coordinates are in a plane. The Z coordinate is perpendicular to the plane. In one aspect of the present invention, the X-Y coordinates are substantially perpendicular to gravity and the Z coordinate is substantially parallel to gravity. This need not be the case, however, particularly if the biochip need not be level for operation or if a gravity free or gravity reduced environment is present. Several particle positioning means are disclosed herein, such as but not limited to dielectric structures, dielectric focusing structures, quadropole electrode structures, electrorotation structures, traveling wave dielectrophoresis structures, concentric electrode structures, spiral electrode structures, circular electrode structures, square electrode structures, particle switch structures, electromagnetic structures, DC electric field induced fluid motion structure, acoustic structures, negative pressure structures and the like. A “dielectric focusing structure” refers to a structure that is on or within a biochip or a chamber that is capable of modulating the position of a particle in the X-Y or X-Y-Z coordinates of a biochip using dielectric forces or dielectrophoretic forces.

A “horizontal positioning means” refers to a particle positioning means that can position a particle in the X-Y coordinates of a biochip or chamber wherein the Z coordinate is substantially defined by gravity.

A “vertical positioning means” refers to a particle positioning means that can position a particle in the Z coordinate of a biochip or chamber wherein the Z coordinate is substantially defined by gravity.

A “quadropole electrode structure” refers to a structure that includes four electrodes arranged around a locus such as a hole, capillary or needle on a biochip and is on or within a biochip or a chamber that is capable of modulating the position of a particle in the X-Y or X-Y-Z coordinates of a biochip using dielectrophoretic forces or dielectric forces generated by such quadropole electrode structures.

An “electrorotation structure” refers to a structure that is on or within a biochip or a chamber that is capable of producing a rotating electric field in the X-Y or X-Y-Z coordinates that can rotate a particle. Preferred electrorotation structures include a plurality of electrodes that are energized using phase offsets, such as 360/N degrees, where N represents the number of electrodes in the electroroation structure (see generally U.S. patent application Ser. No. 09/643,362 entitled “Apparatus and Method for High Throughput Electrorotation Analysis” filed Aug. 22, 2000, naming Jing Cheng et al. as inventors). A rotating electrode structure can also produce dielectrophoretic forces for positioning particles to certain locations under appropriate electric signal or excitation. For example, when N=4 and electrorotation structure corresponds to a quadropole electrode structure.

A “traveling wave dielectrophoresis structure” refers to a structure that is on or within a biochip or a chamber that is capable of modulating the position of a particle in the X-Y or X-Y-Z coordinates of a biochip using traveling wave dielectrophoretic forces (see generally U.S. patent application Ser. No. 09/686,737 filed Oct. 10, 2000, to Xu, Wang, Cheng, Yang and Wu; and U.S. application Ser. No. 09/678,263, entitled “Apparatus for Switching and Manipulating Particles and Methods of Use Thereof” filed on Oct. 3, 2000 and naming as inventors Xiaobo Wang, Weiping Yang, Junquan Xu, Jing Cheng, and Lei Wu).

A “concentric circular electrode structure” refers to a structure having multiple concentric circular electrodes that are on or within a biochip or a chamber that is capable of modulating the position of a particle in the X-Y or X-Y-Z coordinates of a biochip using dielectrophoretic forces.

A “spiral electrode structure” refers to a structure having multiple parallel spiral electrode elements that is on or within a biochip or a chamber that is capable of modulating the position of a particle in the X-Y or X-Y-Z coordinates of a biochip using dielectric forces.

A “square spiral electrode structure” refers to a structure having multiple parallel square spiral electrode elements that are on or within a biochip or a chamber that is capable of modulating the position of a particle in the X-Y or X-Y-Z coordinates of a biochip using dielectrophoretic or traveling wave dielectrophoretic forces.

A “particle switch structure” refers to a structure that is on or within a biochip or a chamber that is capable of transporting particles and switching the motion direction of a particle or particles in the X-Y or X-Y-Z coordinates of a biochip. The particle switch structure can modulate the direction that a particle takes based on the physical properties of the particle or at the will of a programmer or operator (see, generally U.S. application Ser. No. 09/678,263, entitled “Apparatus for Switching and Manipulating Particles and Methods of Use Thereof” filed on Oct. 3, 2000 and naming as inventors Xiaobo Wang, Weiping Yang, Junquan Xu, Jing Cheng, and Lei Wu.

An “electromagnetic structure” refers to a structure that is on or within a biochip or a chamber that is capable of modulating the position of a particle in the X-Y or X-Y-Z coordinates of a biochip using electromagnetic forces. See generally U.S. patent application Ser. No. 09/685,410 filed Oct. 10, 2000, to Wu, Wang, Cheng, Yang, Zhou, Liu and Xu and WO 00/54882 published Sep. 21, 2000 to Zhou, Liu, Chen, Chen, Wang, Liu, Tan and Xu.

A “DC electric field induced fluid motion structure” refers to a structure that is on or within a biochip or a chamber that is capable of modulating the position of a particle in the X-Y or X-Y-Z coordinates of a biochip using DC electric field that produces a fluidic motion.

An “electroosomosis structure” refers to a structure that is on or within a biochip or a chamber that is capable of modulating the position of a particle in the X-Y or X-Y-Z coordinates of a biochip using electroosmotic forces. Preferably, an electroosmosis structure can modulate the positioning of a particle such as a cell or fragment thereof with an ion transport measuring means such that the particle's seal (or the particle's sealing resistance) with such ion transport measuring means is increased.

An “acoustic structure” refers to a structure that is on or within a biochip or a chamber that is capable of modulating the position of a particle in the X-Y or X-Y-Z coordinates of a biochip using acoustic forces. In one aspect of the present invention, the acoustic forces are transmitted directly or indirectly through an aqueous solution to modulate the positioning of a particle. Preferably, an acoustic structure can modulate the positioning of a particle such as a cell or fragment thereof with an ion transport measuring means such that the particle's seal with such ion transport measuring means is increased.

A “negative pressure structure” refers to a structure that is on or within a biochip or a chamber that is capable of modulating the position of a particle in the X-Y or X-Y-Z coordinates of a biochip using negative pressure forces, such as those generated through the use of pumps or the like. Preferably, a negative pressure structure can modulate the positioning of a particle such as a cell or fragment thereof with an ion transport measuring means such that the particle's seal with such ion transport measuring means is increased.

“Dielectrophoresis” is the movement of polarized particles in electrical fields of nonuniform strength. There are generally two types of dielectrophoresis, positive dielectrophoresis and negative dielectrophoresis. In positive dielectrophoresis, particles are moved by dielectrophoretic forces toward the strong field regions. In negative dielectrophoresis, particles are moved by dielectrophoretic forces toward weak field regions. Whether moieties exhibit positive or negative dielectrophoresis depends on whether particles are more or less polarizable than the surrounding medium.

A “dielectrophoretic force” is the force that acts on a polarizable particle in an AC electrical field of non-uniform strength. The dielectrophoretic force \vec{F}_{DEP} acting on a particle of radius r subjected to a non-uniform electrical field can be given, under the dipole approximation, by:
\vec{F}_{DEP}=2πεₘr³χ_{DEP}∇E²ᵣₘₛ
where Eᵣₘₛ is the RMS value of the field strength, the symbol ∇ is the symbol for gradient-operation, εₘ is the dielectric permittivity of the medium, and χ_{DEP} is the particle polarization factor, given by:

$${χ}_{DEP} = Re ( \frac{{ɛ}_{p}^{*} - {ɛ}_{m}^{*}}{{ɛ}_{p}^{*} + 2 {ɛ}_{m}^{*}} ) ,$$

“Re” refers to the real part of the “complex number”. The symbol εₓ*=εₓ−jσₓ/2πf is the complex permittivity (of the particle x=p, and the medium x=m) and j=\sqrt{−1}. The parameters εₚ and σₚ are the effective permittivity and conductivity of the particle, respectively. These parameters may be frequency dependent. For example, a typical biological cell will have frequency dependent, effective conductivity and permittivity, at least, because of cytoplasm membrane polarization. Particles such as biological cells having different dielectric properties (as defined by permittivity and conductivity) will experience different dielectrophoretic forces. The dielectrophoretic force in the above equation refers to the simple dipole approximation results. However, the dielectrophoretic force utilized in this application generally refers to the force generated by non-uniform electric fields and is not limited by the dipole simplification. The above equation for the dielectrophoretic force can also be written as
\vec{F}_{DEP}=2πεₘr³χ_{DEP} V²∇p(x,y,z)
where p(x,y,z) is the square-field distribution for a unit-voltage excitation (Voltage V=1 V) on the electrodes, V is the applied voltage.

“Traveling-wave dielectrophoretic (TW-DEP) force” refers to the force that is generated on particles or molecules due to a traveling-wave electric field. An ideal traveling-wave field is characterized by the distribution of the phase values of AC electric field components, being a linear function of the position of the particle. In this case the traveling wave dielectrophoretic force \vec{F}_{TW-DEP} on a particle of radius r subjected to a traveling wave electrical field E=E cos(2π(ft−z/λ₀)\vec{a}ₓ (i.e., a x-direction field is traveling along the z-direction) is given, again, under the dipole approximation, by

$${{F}^{->}}_{TW - DEP} = - \frac{4 {π}^{2} {ɛ}_{m}}{{λ}_{0}} {r}^{3} {ζ}_{TW - DEP} {E}^{2} \cdot {{a}^{->}}_{z}$$

where E is the magnitude of the field strength, εₘ is the dielectric permittivity of the medium. ζ_{TW-DEP} is the particle polarization factor, given by

$${ζ}_{TW - DEP} = Im ( \frac{{ɛ}_{p}^{*} - {ɛ}_{m}^{*}}{{ɛ}_{p}^{*} + 2 {ɛ}_{m}^{*}} ) ,$$

“Im” refers to the imaginary part of the “complex number”. The symbol εₓ*=εₓ−jσₓ/2πf is the complex permittivity (of the particle x=p, and the medium x=m). The parameters εₚ and σₚ are the effective permittivity and conductivity of the particle, respectively. These parameters may be frequency dependent.

A traveling wave electric field can be established by applying appropriate AC signals to the microelectrodes appropriately arranged on a chip. For generating a traveling-wave-electric field, it is necessary to apply at least three types of electrical signals each having a different phase value. An example to produce a traveling wave electric field is to use four phase-quardrature signals (0, 90, 180 and 270 degrees) to energize four linear, parallel electrodes patterned on the chip surfaces. Such four electrodes may be used to form a basic, repeating unit. Depending on the applications, there may be more than two such units that are located next to each other. This will produce a traveling-electric field in the spaces above or near the electrodes. As long as electrode elements are arranged following certain spatially sequential orders, applying phase-sequenced signals will result in establishing traveling electrical fields in the region close to the electrodes.

“Electric field pattern” refers to the field distribution in space or in a region of interest. An electric field pattern is determined by many parameters, including the frequency of the field, the magnitude of the field, the magnitude distribution of the field, and the distribution of the phase values of the field components, the geometry of the electrode structures that produce the electric field, and the frequency and/or magnitude modulation of the field.

“Dielectric properties” of a particle are properties that determine, at least in part, the response of a particle to an electric field. The dielectric properties of a particle include the effective electric conductivity of a particle and the effective electric permittivity of a particle. For a particle of homogeneous composition, for example, a polystyrene bead, the effective conductivity and effective permittivity are independent of the frequency of the electric field at least for a wide frequency range (e.g. between 1 Hz to 100 MHz). Particles that have a homogeneous bulk composition may have net surface charges. When such charged particles are suspended in a medium, electrical double layers may form at the particle/medium interfaces. Externally applied electric field may interact with the electrical double layers, causing changes in the effective conductivity and effective permittivity of the particles. The interactions between the applied field and the electrical double layers are generally frequency dependent. Thus, the effective conductivity and effective permittivity of such particles may be frequency dependent. For moieties of nonhomogeneous composition, for example, a cell, the effective conductivity and effective permittivity are values that take into account the effective conductivities and effective permittivities of both the membrane and internal portion of the cell, and can vary with the frequency of the electric field. In addition, the dielectrophoretic force experience by a particle in an electric field is dependent on its size; therefore, the overall size of particle is herein considered to be a dielectric property of a particle. Properties of a particle that contribute to its dielectric properties include but are not limited to the net charge on a particle; the composition of a particle (including the distribution of chemical groups or moieties on, within, or throughout a particle); size of a particle; surface configuration of a particle; surface charge of a particle; and the conformation of a particle. Particles can be of any appropriate shape, such as geometric or non-geometric shapes. For example, particles can be spheres, non-spherical, rough, smooth, have sharp edges, be square, oblong or the like.

“Magnetic forces” refer to the forces acting on a particle due to the application of a magnetic field. In general, particles have to be magnetic or paramagnetic when sufficient magnetic forces are needed to manipulate particles. For a typical magnetic particle made of super-paramagnetic material, when the particle is subjected to a magnetic field \vec{B}, a magnetic dipole \vec{μ} is induced in the particle

$$\begin{matrix}{μ}^{->} = {V}_{p} ( {χ}_{p} - {χ}_{m} ) \frac{{B}^{->}}{{μ}_{m}} , \\ = {V}_{p} ( {χ}_{p} - {χ}_{m} ) {{H}^{->}}_{m}\end{matrix}$$

where Vₚ is the particle volume, χₚ and χₘ are the volume susceptibility of the particle and its surrounding medium, μm is the magnetic permeability of medium, \vec{H}ₘ is the magnetic field strength. The magnetic force \vec{F}_{magnetic} acting on the particle is determined, under the dipole approximation, by the magnetic dipole moment and the magnetic field gradient:
\vec{F}_{magnetic}=−0.5 Vₚ(χₚ−χₘ)\vec{H}ₘ●∇\vec{B}ₘ,
where the symbols “●” and “∇” refer to dot-product and gradient operations, respectively. Whether there is magnetic force acting on a particle depends on the difference in the volume susceptibility between the particle and its surrounding medium. Typically, particles are suspended in a liquid, non-magnetic medium (the volume susceptibility is close to zero) thus it is necessary to utilize magnetic particles (its volume susceptibility is much larger than zero). The particle velocity v_{particle} under the balance between magnetic force and viscous drag is given by:

$${v}_{particle} = \frac{{{F}^{->}}_{magnetic}}{6 π r {η}_{m}}$$

where r is the particle radius and ηₘ is the viscosity of the surrounding medium.

As used herein, “manipulation” refers to moving or processing of the particles, which results in one-, two- or three-dimensional movement of the particle, in a chip format, whether within a single chip or between or among multiple chips. Non-limiting examples of the manipulations include transportation, focusing, enrichment, concentration, aggregation, trapping, repulsion, levitation, separation, isolation or linear or other directed motion of the particles. For effective manipulation, the binding partner and the physical force used in the method should be compatible. For example, binding partner such as microparticles that can be bound with particles, having magnetic properties are preferably used with magnetic force. Similarly, binding partners having certain dielectric properties, for example, plastic particles, polystyrene microbeads, are preferably used with dielectrophoretic force.

A “sample” is any sample from which particles are to be separated or analyzed. A sample can be from any source, such as an organism, group of organisms from the same or different species, from the environment, such as from a body of water or from the soil, or from a food source or an industrial source. A sample can be an unprocessed or a processed sample. A sample can be a gas, a liquid, or a semi-solid, and can be a solution or a suspension. A sample can be an extract, for example a liquid extract of a soil or food sample, an extract of a throat or genital swab, or an extract of a fecal sample. Samples are can include cells or a population of cells. The population of cells can be a mixture of different cells or a population of the same cell or cell type, such as a clonal population of cells. Cells can be derived from a biological sample from a subject, such as a fluid, tissue or organ sample. In the case of tissues or organs, cells in tissues or organs can be isolated or separated from the structure of the tissue or organ using known methods, such as teasing, rinsing, washing, passing through a grating and treatment with proteases. Samples of any tissue or organ can be used, including mesodermally derived, endodermally derived or ectodermally derived cells. Particularly preferred types of cells are from the heart and blood. Cells include but are not limited to suspensions of cells, cultured cell lines, recombinant cells, infected cells, eukaryotic cells, prokaryotic cells, infected with a virus, having a phenotype inherited or acquired, cells having a pathological status including a specific pathological status or complexed with biological or non-biological entities.

“Separation” is a process in which one or more components of a sample is spatially separated from one or more other components of a sample or a process to spatially redistribute particles within a sample such as a mixture of particles, such as a mixture of cells. A separation can be performed such that one or more particles is translocated to one or more areas of a separation apparatus and at least some of the remaining components are translocated away from the area or areas where the one or more particles are translocated to and/or retained in, or in which one or more particles is retained in one or more areas and at least some or the remaining components are removed from the area or areas. Alternatively, one or more components of a sample can be translocated to and/or retained in one or more areas and one or more particles can be removed from the area or areas. It is also possible to cause one or more particles to be translocated to one or more areas and one or more moieties of interest or one or more components of a sample to be translocated to one or more other areas. Separations can be achieved through the use of physical, chemical, electrical, or magnetic forces. Examples of forces that can be used in separations include but are not limited to gravity, mass flow, dielectrophoretic forces, traveling-wave dielectrophoretic forces, and electromagnetic forces.

“Capture” is a type of separation in which one or more particles is retained in one or more areas of a chip. In the methods of the present application, a capture can be performed when physical forces such as dielectrophoretic forces or electromagnetic forces are acted on the particle and direct the particle to one or more areas of a chip.

An “assay” is a test performed on a sample or a component of a sample. An assay can test for the presence of a component, the amount or concentration of a component, the composition of a component, the activity of a component, the electrical properties of an ion transport protein, etc. Assays that can be performed in conjunction with the compositions and methods of the present invention include, but not limited to, biochemical assays, binding assays, cellular assays, genetic assays, ion transport assay, gene expression assays and protein expression assays.

A “binding assay” is an assay that tests for the presence or the concentration of an entity by detecting binding of the entity to a specific binding member, or an assay that tests the ability of an entity to bind another entity, or tests the binding affinity of one entity for another entity. An entity can be an organic or inorganic molecule, a molecular complex that comprises, organic, inorganic, or a combination of organic and inorganic compounds, an organelle, a virus, or a cell. Binding assays can use detectable labels or signal generating systems that give rise to detectable signals in the presence of the bound entity. Standard binding assays include those that rely on nucleic acid hybridization to detect specific nucleic acid sequences, those that rely on antibody binding to entities, and those that rely on ligands binding to receptors.

A “biochemical assay” is an assay that tests for the composition of or the presence, concentration, or activity of one or more components of a sample.

A “cellular assay” is an assay that tests for or with a cellular process, such as, but not limited to, a metabolic activity, a catabolic activity, an ion transport function or property, an intracellular signaling activity, a receptor-linked signaling activity, a transcriptional activity, a translational activity, or a secretory activity.

An “ion transport assay” is an assay useful for determining ion transport functions or properties and testing for the abilities and properties of chemical entities to alter ion transport functions. Preferred ion transport assays include electrophysiology-based methods which include, but are not limited to patch clamp recording, whole cell recording, perforated patch or whole cell recording, vesicle recording, outside out and inside out recording, single channel recording, artificial membrane channel recording, voltage gated ion transport recording, ligand gated ion transport recording, stretch activated (fluid flow or osmotic) ion transport recording, and recordings on energy requiring ion transporters (such as ATP), non energy requiring transporters, and channels formed by toxins such a scorpion toxins, viruses, and the like. See, generally Neher and Sakman, Scientific American 266:44-51 (1992); Sakmann and Heher, Ann. Rev. Physiol. 46:455-472 (1984); Cahalan and Neher, Methods in Enzymology 207:3-14 (1992); Levis and Rae, Methods in Enzymology 207:14-66 (1992); Armstrong and Gilly, Methods in Enzymology 207:100-122 (1992); Heinmann and Conti, Methods in Enzymology 207:131-148 (1992); Bean, Methods in Enzymology 207:181-193 (1992); Leim et al., Neurosurgery 36:382-392 (1995); Lester, Ann. Rev. Physiol 53:477-496 (1991); Hamill and McBride, Ann. Rev. Physiol 59:621-631 (1997); Bustamante and Varranda, Brazilian Journal 31:333-354 (1998); Martinez-Pardon and Ferrus, Current Topics in Developmental Biol. 36:303-312 (1998); Herness, Physiology and Behavior 69:17-27 (2000); Aston-Jones and Siggins, www.acnp.org/GA/GN40100005/CH005.html (Feb. 8, 2001); U.S. Pat. No. 6,117,291; U.S. Pat. No. 6,107,066; U.S. Pat. No. 5,840,041 and U.S. Pat. No. 5,661,035; Boulton et al., Patch-Clamp Applications and Protocols, Neuromethods V. 26 (1995), Humana Press, New Jersey; Ashcroft, Ion Channels and Disease, Cannelopathies, Academic Press, San Diego (2000); Sakmann and Neher, Single Channel Recording, second edition, Plenuim Press, New York (1995) and Soria and Cena, Ion Channel Pharmacology, Oxford University Press, New York (1998), each of which is incorporated by reference herein in their entirety.

An “electrical seal” refers to a high-resistance engagement between a particle such as a cell or cell membrane and an ion transport measuring means, such as a hole, capillary or needle of a chip or device of the present invention. Preferred resistance of such an electrical seal is between about 1 mega ohm and about 100 giga ohms, but that need not be the case. Generally, a large resistance results in decreased noise in the recording signals. For specific types of ion channels (with different magnitude of recording current) appropriate electric sealing in terms of mega ohms or giga ohms can be used.

An “acid” includes acid and acidic compounds and solutions that have a pH of less than 7 under conditions of use.

A “base” includes base and basic compounds and solutions that have a pH of greater than 7 under conditions of use.

“More electronegative” means having a higher density of negative charge. In the methods of the present invention, a chip or ion transport measuring means that is more electronegative has a higher density of negative surface charge.

An “electrolyte bridge” is a liquid (such as a solution) or a solid (such as an agar salt bridge) conductive connection with at least one component of the electrolyte bridge being an electrolyte so that the bridge can pass current with no or low resistance.

A “ligand gated ion transport” refers to ion transporters such as ligand gated ion channels, including extracellular ligand gated ion channels and intracellular ligand gated ion channels, whose activity or function is activated or modulated by the binding of a ligand. The activity or function of ligand gated ion transports can be detected by measuring voltage or current in response to ligands or test chemicals. Examples include but are not limited to GABA_{A}, strychnine-sensitive glycine, nicotinic acetylcholine (Ach), ionotropic glutamate (iGlu), and 5-hydroxytryptamine₃ (5-HT₃) receptors.

A “voltage gated ion transport” refers to ion transporters such as voltage gated ion channels whose activity or function is activated or modulated by voltage. The activity or function of voltage gated ion transports can be detected by measuring voltage or current in response to different commanding currents or voltages respectively. Examples include but are not limited to voltage dependent Na⁺ channels.

“Perforated patch clamp” refers to the use of perforation agents such as but not limited to nystatin or amphotericin B to form pores or perforations in membranes that are preferably ion-conducting, which allows for the measurement of current, including whole cell current.

An “electrode” is a structure of highly electrically conductive material. A highly conductive material is a material with conductivity greater than that of surrounding structures or materials. Suitable highly electrically conductive materials include metals, such as gold, chromium, platinum, aluminum, and the like, and can also include nonmetals, such as carbon, conductive liquids and conductive polymers. An electrode can be any shape, such as rectangular, circular, castellated, etc. Electrodes can also comprise doped semi-conductors, where a semi-conducting material is mixed with small amounts of other “impurity” materials. For example, phosphorous-doped silicon may be used as conductive materials for forming electrodes.

A “channel” is a structure with a lower surface and at least two walls that extend upward from the lower surface of the channel, and in which the length of two opposite walls is greater than the distance between the two opposite walls. A channel therefore allows for flow of a fluid along its internal length. A channel can be covered (a “tunnel”) or open.

“Continuous flow” means that fluid is pumped or injected into a chamber of the present invention continuously during an assay or separation process, or before or after an assay or separation process. This allows for components of a sample or solution that are not selectively retained on a chip to be flushed out of the chamber.

“Binding partner” refers to any substances that both bind to the moieties with desired affinity or specificity and are manipulatable with the desired physical force(s). Non-limiting examples of the binding partners include cells, cellular organelles, viruses, particles, microparticles or an aggregate or complex thereof, or an aggregate or complex of molecules.

A “specific binding member” is one of two different molecules having an area on the surface or in a cavity that specifically binds to and is thereby defined as complementary with a particular spatial and polar organization of the other molecule. A specific binding member can be a member of an immunological pair such as antigen-antibody, can be biotin-avidin or biotin streptavidin, ligand-receptor, nucleic acid duplexes, IgG-protein A, DNA-DNA, DNA-RNA, RNA-RNA, and the like.

A “nucleic acid molecule” is a polynucleotide. A nucleic acid molecule can be DNA, RNA, or a combination of both. A nucleic acid molecule can also include sugars other than ribose and deoxyribose incorporated into the backbone, and thus can be other than DNA or RNA. A nucleic acid can comprise nucleobases that are naturally occurring or that do not occur in nature, such as xanthine, derivatives of nucleobases, such as 2-aminoadenine, and the like. A nucleic acid molecule of the present invention can have linkages other than phosphodiester linkages. A nucleic acid molecule of the present invention can be a peptide nucleic acid molecule, in which nucleobases are linked to a peptide backbone. A nucleic acid molecule can be of any length, and can be single-stranded, double-stranded, or triple-stranded, or any combination thereof. The above described nucleic acid molecules can be made by a biological process or chemical synthesis or a combination thereof.

A “detectable label” is a compound or molecule that can be detected, or that can generate readout, such as fluorescence, radioactivity, color, chemiluminescence or other readouts known in the art or later developed. Such labels can be, but are not limited to, photometric, colorimetric, radioactive or morphological such as changes of cell morphology that are detectable, such as by optical methods. The readouts can be based on fluorescence, such as by fluorescent labels, such as but not limited to, Cy-3, Cy-5, phycoerythrin, phycocyanin, allophycocyanin, FITC, rhodamine, or lanthanides; and by fluorescent proteins such as, but not limited to, green fluorescent protein (GFP). The readout can be based on enzymatic activity, such as, but not limited to, the activity of beta-galactosidase, beta-lactamase, horseradish peroxidase, alkaline phosphatase, or luciferase. The readout can be based on radioisotopes (such as ³³P, ³H , ¹⁴C, ³⁵S, ¹²⁵I, ³²P or ¹³¹I). A label optionally can be a base with modified mass, such as, for example, pyrimidines modified at the C5 position or purines modified at the N7 position. Mass modifying groups can be, for examples, halogen, ether or polyether, alkyl, ester or polyester, or of the general type XR, wherein X is a linking group and R is a mass-modifying group. One of skill in the art will recognize that there are numerous possibilities for mass-modifications useful in modifying nucleic acid molecules and oligonucleotides, including those described in Oligonucleotides and Analogues: A Practical Approach, Eckstein, ed. (1991) and in PCT/US94/00193.

A “signal producing system” may have one or more components, at least one component usually being a labeled binding member. The signal producing system includes all of the reagents required to produce or enhance a measurable signal including signal producing means capable of interacting with a label to produce a signal. The signal producing system provides a signal detectable by external means, often by measurement of a change in the wavelength of light absorption or emission. A signal producing system can include a chromophoric substrate and enzyme, where chromophoric substrates are enzymatically converted to dyes, which absorb light in the ultraviolet or visible region, phosphors or fluorescers. However, a signal producing system can also provide a detectable signal that can be based on radioactivity or other detectable signals.

The signal producing system can include at least one catalyst, usually at least one enzyme, and can include at least one substrate, and may include two or more catalysts and a plurality of substrates, and may include a combination of enzymes, where the substrate of one enzyme is the product of the other enzyme. The operation of the signal producing system is to produce a product that provides a detectable signal at the predetermined site, related to the presence of label at the predetermined site.

In order to have a detectable signal, it may be desirable to provide means for amplifying the signal produced by the presence of the label at the predetermined site. Therefore, it will usually be preferable for the label to be a catalyst or luminescent compound or radioisotope, most preferably a catalyst. Preferably, catalysts are enzymes and coenzymes that can produce a multiplicity of signal generating molecules from a single label. An enzyme or coenzyme can be employed which provides the desired amplification by producing a product, which absorbs light, for example, a dye, or emits light upon irradiation, for example, a fluorescer. Alternatively, the catalytic reaction can lead to direct light emission, for example, chemiluminescence. A large number of enzymes and coenzymes for providing such products are indicated in U.S. Pat. No. 4,275,149 and U.S. Pat. No. 4,318,980, which disclosures are incorporated herein by reference. A wide variety of non-enzymatic catalysts that may be employed are found in U.S. Pat. No. 4,160,645, issued Jul. 10, 1979, the appropriate portions of which are incorporated herein by reference.

The product of the enzyme reaction will usually be a dye or fluorescer. A large number of illustrative fluorescers are indicated in U.S. Pat. No. 4,275,149, which is incorporated herein by reference.

Other technical terms used herein have their ordinary meaning in the art that they are used, as exemplified by a variety of technical dictionaries.

Introduction

The present invention recognizes that using direct detection methods to determine an ion transport function or property, such as patch-clamps, is preferable to using indirect detection methods, such as fluorescence-based detection systems. The present invention provides biochips and methods of use that allow for the direct detection of one or more ion transport functions or properties using chips and devices that can allow for automated detection of one or more ion transport functions or properties. These biochips and methods of use thereof are particularly appropriate for automating the detection of ion transport functions or properties, particularly for screening purposes.

As a non-limiting introduction to the breath of the present invention, the present invention includes several general and useful aspects, including:
1) a biochip device for ion transport measurement that comprises at least one upper chamber piece and at least one biochip that comprises at least one ion transport measuring means. The device can comprise one or more conduits that provide an electrolyte bridge to at least one electrode.
2) a biochip ion transport measuring device having one or more flow-through lower chambers.
3) a biochip devices adapted for a microscope stage.
4) methods of making an upper piece for a biochip device for ion transport measurement.
5) methods for making chips comprising ion transport measurement holes using laser drilling techniques.
6) devices that include an inverted chip for ion transport measurement.
7) methods of treating ion transport measuring chips to enhance their sealing properties.
8) a method to measure surface energy, such as on the surface of a chemically-treated ion transport measurement biochip.
9) substrates, biochips, cartridges, apparatuses, and/or devices comprising ion transport measuring means with enhanced electric seal properties.
10) methods for storing the substrates, biochips, cartridges, apparatuses, and/or devices comprising ion transport measuring means with enhanced electrical seal properties.
11) methods for shipping the substrates, biochips, cartridges, apparatuses, and/or devices comprising ion transport measuring means with enhanced electrical seal properties.
12) methods for assembling devices and cartridges of the present invention using UV adhesives.
13) a method of producing ion transport measuring chips by fabricating them as detachable units of a larger sheet.
14) a method of producing high density ion transport measuring chips.
15) a biochip device for ion transport measurement comprising fluidic channel upper and lower chambers.
16) methods of preparing cells for ion transport measurement.
17) a software program logic that controls a pressure control profile to direct an ion transport measurement apparatus to achieve and maintain a high-resistance electrical seal.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

I. Device for Ion Transport Measurement

The present invention comprises devices for ion transport measurement and components of ion transport measuring devices that reduce the costs of manufacture and use and are efficient and convenient to use. The devices of the present invention are also designed for maximum versatility, providing for different assay formats within the same basic design.

In some aspects, the present invention contemplates devices and apparatuses that have parts that are manufactured separately and can be assembled to form ion transport measuring devices that have at least one, and preferably multiple, ion transport measuring units, each of which comprises an upper chamber and at least a portion of a biochip that comprises an ion transport measuring means that during use of the device can connect the upper chamber with a lower chamber. An ion transport measuring device of the present invention can further comprise at least a portion of at least one lower chamber that is connected to one or more upper chambers of the device via an ion transport measuring means of the chip. These devices comprising ion channel measuring units can be assembled before the assay procedure, and pieces that make up the device can be reversibly or irreversibly attached to one another.

In many preferred aspects of the present invention, a device or one or more parts of a device can be removed from an apparatus and can be disposable after a single use (for example, a chip comprising ion transport measuring means; one or more upper chambers designed to contain cells), and can engage one or more parts of an ion transport measuring device or apparatus that can be permanent and reusable (for example, at least a portion of a lower chamber; one or more electrodes) For example, in some aspects of the present invention, devices comprising one or more upper chamber pieces and at least one biochip (called cartridges) are single-use and disposable, and lower chamber pieces, one or more electrodes, and platforms or lower base pieces are reusable. In these aspects, upper chamber pieces and biochips can be reversibly or irreversibly attached to one another during use of the device or apparatus, and these attached upper chamber/biochip devices can be reversibly attached to or contacted with lower chamber pieces, conduits, or electrodes.

In one embodiment, the present invention contemplates an ion transport measuring device in the form of a cartridge that comprises an upper chamber piece that comprises at least one well that is open at its upper and lower ends, and a biochip that comprises at least one ion transport measuring means. The chip is reversibly or irreversibly attached to the bottom of the upper chamber piece such that each of the one or more upper wells is in register with one of the one or more ion transport measuring means, providing one or more independent upper chambers each in contact with a single ion transport measuring means. The chip can be in direct or indirect contact with the upper chamber piece.

In a cartridge in which an upper chamber piece is in indirect contact with an attached chip, a spacer or gasket, for example, can be between the upper chamber piece and the chip. A chip can be in direct contact with an upper chamber piece of a cartridge if it is attached during molding of the cartridge, by heat sealing, or by adhesives, for example. Attachment of a chip to an upper chamber piece to make a cartridge can be performed by a machine, and can be automated.

A chip can also be intergral to an upper chamber piece in a cartridge or device of the present invention, where the chip forms or is part of the lower surface of the upper chamber piece that can comprise, for example, glass or one or more plastics.

Preferably a biochip that is part of an ion transport measuring device of the present invention comprises multiple holes used as ion transport measuring means, and an upper chamber piece comprises multiple upper chambers such that each of the upper chambers is in register with one of the ion transport measuring means of the chip. For example, preferred devices and apparatuses for ion transport measurement can have two or more, four or more, eight or more, or sixteen or more ion transport measuring units and comprise upper chamber pieces comprising a corresponding number of upper chambers. For example, ion transport measuring devices can have sixteen, twenty-four, forty-eight, ninety-six or more ion transport measuring units and comprise upper chamber pieces comprising a corresponding number of upper chambers.

The upper chambers or wells can be any shape or size. Typically, the upper chambers will be in the form of wells which can be tapered or non-tapered. The wells of an upper chamber piece that can be part of an ion transport measuring device preferably can hold a volume of between about 0.5 microliters and about 5 milliliters or more, more preferably between about 10 microliters and about 2 milliliters, and more preferably yet between about 25 microliters and about 1 milliliter. The upper diameter of a well can be from about 0.05 millimeter to about 20 millimeters or more, and is preferably between about 2 millimeters and about 10 millimeters or more. The depth, or height of a well can vary from about 0.01 to about 25 millimeters or more, and more preferably will be from about 2 millimeters to about 10 millimeters. In designs in which the upper well or wells are tapered, the well can be tapered downward at an angle of from about 0.1 degree to about 89 degrees from vertical, and preferably from about 5 degrees to about 60 degrees from vertical. The well can be tapered at one or more ends, or throughout the circumference of the well.

An upper chamber piece can be made of any suitable material, (for example, one or more plastics, one or more polymers, one or more ceramic, one or more waxes, silicon, or glass) but for ease of manufacturing is preferably made of a moldable plastic, such as, for example, polysulfone, polyallomer, polyethylene, polyimide, polypropylene, polystyrene, polycarbonate, cylco olefin polymer (such as, for example, ZEONOR®), polyphenylene ether/PPO or modified polyphenylene oxide (such as, for example, NORYL®), or composite polymers. In some aspects, base resistant plastics such as polystyrene, cylco olefin polymers (such as, for example, ZEONOR®), polyphenylene ether/PPO or modified polyphenylene oxide (such as, for example, NORYL®), can be preferred.

An upper chamber piece can optionally comprise one or more electrodes. An upper chamber piece that comprises multiple upper chambers can comprise multiple electrodes, where each well contacts an independent electrode (such as, for example, independent recording electrodes). In an alternative design, an upper chamber piece can contain or contact at least a portion of a single electrode (which can be, for example, a reference electrode) that contacts all of the upper chambers of the device. In designs in which the upper chamber piece does not comprise one or more electrodes, the upper chamber piece can optionally be used as part of an apparatus for ion transport measurement in which one or more electrodes can be introduced into one or more upper chambers (such as, for example, introduced via a conduit that can be connected to or can be inserted into one or more chambers). In an alternative configuration, conduits connected with or introduced into one or more upper chambers can, during the use of the apparatus, be filled with a measuring solution and provide electrolyte bridges to one or more electrodes.

The chip can be reversibly or irreversibly attached the lower surface of an upper chamber piece to form a cartridge by any feasible means that provides a fluid-impermeable seal between the chip and the upper chamber piece, such as by adhesives or by pressure mounting. The chip of the assembled cartridge can be in direct or indirect contact with an upper chamber piece. Preferably but optionally, the chip is irreversibly attached to the upper chamber piece, such as by one or more adhesives, to make a cartridge. Such cartridges can optionally single use and disposable. Assembly of a preferred cartridge of the present invention is provided in Example 1.

An upper chamber piece of the present invention can also have features that aid in the manufacture of the piece or assembly of the cartridge. For example, the lower surface of the upper chamber piece can comprise one or more alignment bumps or registration edges on at least one end of the lower side of the piece that allows a chip to be positioned against the lower side of the upper chamber piece such that the ion transport measuring holes of the chip are in register with the wells. Features that facilitate manufacture of an upper chamber piece include one or more sink holes that prevent the piece from deforming through thermal contraction of the piece during the injection molding process, and one or more glue spillage grooves that allow for seepage of excess glue that may be used in attaching a chip to the upper chamber piece. Assembly of a cartridge can be done manually, or by a machine. Preferably but optionally, at least one of the steps in the assembly of a cartridge of the present invention by a machine is automated. For example, a machine may perform one or more of the steps of: picking up a chip from a rack or holder, picking up an upper chamber piece from a rack, platform, shelf, or holder, applying one or more adhesives to an upper chamber piece or a chip, positioning a chip on the bottom of an upper chamber piece so that the ion transport measuring means of the chip are in register with the wells of the upper chamber piece, and allowing or promoting attachment of the chip to the upper chamber piece (such as by treating with UV or heat).

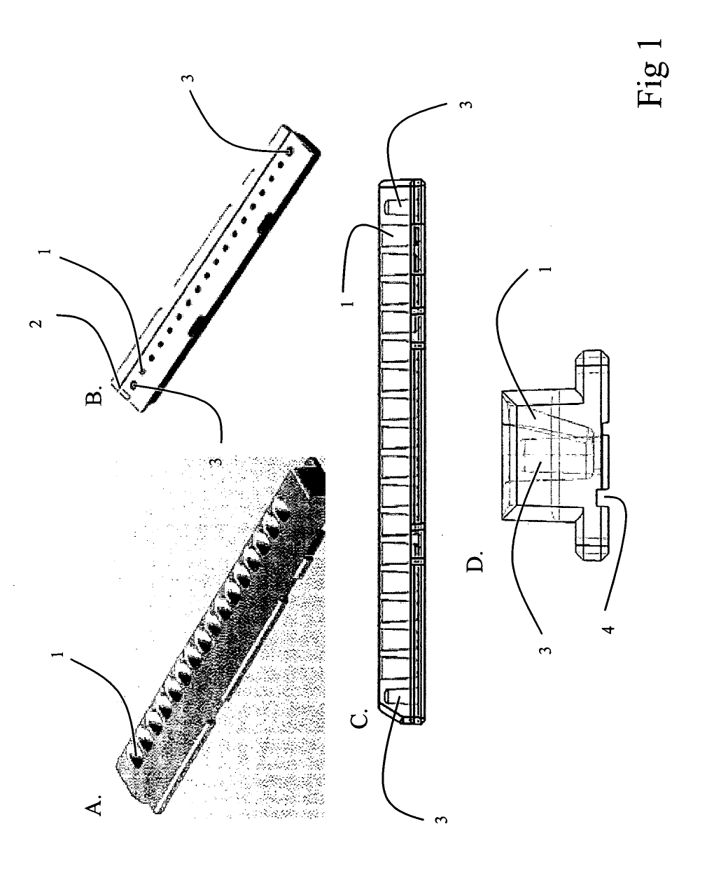

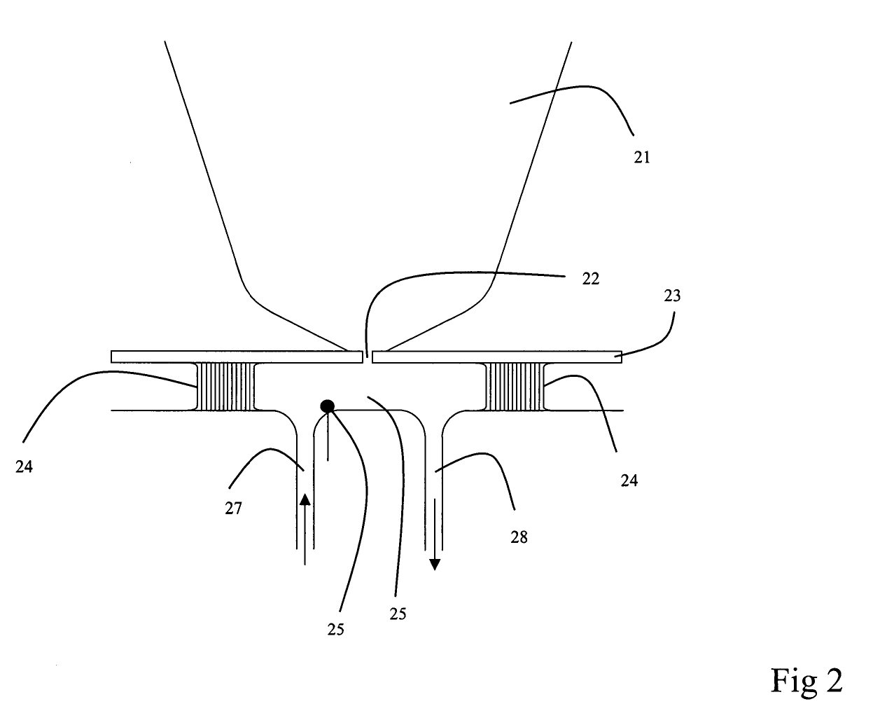

One design of an upper chamber piece is shown in FIG. 1. FIG. 1A depicts a top view of an upper chamber piece having sixteen wells (1) and FIG. 1B depicts a bottom view of the upper chamber piece showing the lower openings of the sixteen wells (1), and also shows the openings of two sinkholes (3). (In an assembled cartridge or device comprising a chip, the chip preferably covers and thereby seals off, the sinkhole openings.) In this design, the wells (1) are tapered such that the upper diameters of the wells (1) (seen in FIG. 1A) are larger than the lower diameters of the wells (1) (seen in FIG. 1B). In FIG. 1C, the upper chamber piece is shown side-on in cross-section, showing the sixteen wells (1) as well as features that increase the efficiency of manufacture of a device, including an alignment bump (2) for chip positioning and sink holes (3) that prevent cave-in of the upper chamber piece due to contraction of the plastic as it cools after molding of the piece. FIG. 1D is an end-on cross-sectional view of the piece showing a well (1) behind a sink hole (3). In FIG. 1D a glue spillage groove (4) is also shown. A glue spillage groove can allow for seepage of an adhesive used to seal a chip to the lower chamber piece to make a cartridge.

A chip used in a device of the present invention is preferably a chip that comprises ion transport measuring means in the form of holes. A chip used in a device of the present invention can comprise glass, silicon, silicon dioxide, quartz, one or more plastics, one or more waxes, or one or more polymers (for example, polydimethylsiloxane (PDMS)), one or more ceramics, or a combination thereof. Methods of fabricating such chips, including methods of fabricating ion transport measuring holes in chips, are disclosed in related applications, including U.S. patent application Ser. No. 10/760,866 (pending), filed Jan. 20, 2004; U.S. patent application Ser. No. 10/642,014, filed Aug. 16, 2003; and U.S. patent application Ser. No. 10/104,300, filed Mar. 22, 2002; each of which is incorporated by reference herein.

A chip used in a device of the present invention is preferably a “K-configuration” chip, but this is not a requirement of the present invention. As described in a later section of this application and in the Examples, K-configuration chips have ion transport measuring holes that comprise a through-hole that is laser drilled through one or more counterbores. A chip used in a device of the present invention is preferably treated to have enhanced sealing properties. Methods of chemically treating ion transport measuring chips, for example with basic solutions, to enhance their ability to form electrical seals with particles such as cells are disclosed herein. A preferred device for ion transport measurement is a cartridge that comprises a K-configuration chip with enhanced electrical sealing properties that is reversibly or irreversibly attached to an upper chamber piece. Preferably, a chip assembled into a device of the present invention has one or more ion transport measuring holes that is able to seal to a cell or particle such that electrical access between the chip and the inside of the cell or particle (or between the chip and the inside of the cell or particle) has an access resistance that (Ra) is less than the seal resistance (R). Preferably, the access resistance of a whole-cell configuration seal that can be formed on the hole of a chip of a device of the present invention is less than 80 MOhm, more preferably less than about 30 MOhm, and more preferably yet, less than about 10 MOhm. Preferably, a chip of a device of the present invention can form a seal with a cell such that the seal has a resistance that is at least 200 MOhm, and more preferably, at least 500 MOhm, and more preferably yet, about 1 GigaOhm or greater. Preferably, a chip of a device of the present invention comprises at least one ion transport measuring means in the form of a through-hole that has been laser-drilled through at least one counterbore, in which at least the surface of the ion transport measuring means has been treated to enhance its electrical sealing properties, and the chip can form a seal between at least one ion transport measuring means and a cell such that the resistance (R) of the seal is at least ten times the access resistance of the seal. More preferably, a chip of a device of the present invention can form a seal with a cell such that the seal resistance is at least twenty times the Ra.

Preferably, a chip comprising laser-drilled ion transport measuring holes is attached to an upper chamber piece in inverted orientation, as described in a later section of this application, such that the laser entrance hole of the ion transport measuring hole is exposed to the upper chambers, but this is not a requirement of the present invention. In the alternative, the chip can be attached to the upper chamber in “upside up” orientation.

A cartridge comprising an upper chamber piece and at least one biochip comprising one or more ion transport measuring means can be assembled into a device that comprises one or more lower chambers in which the one or more lower chambers access at least one upper chamber via a hole in the biochip. A cartridge can engage one or more parts that make up one or more lower chambers, where the one or more lower chambers are directly or indirectly attached to the underside of the chip, and at least one ion transport measuring hole in the chip connects the one or more lower chambers with one or more upper chambers of the device.

For example, a cartridge comprising an upper chamber piece and at least one biochip comprising one or more ion transport measuring means can be assembled with a lower chamber piece that comprises at least a portion of at least one lower chamber. The cartridge can be assembled with a lower chamber piece that comprises at least a portion of a single lower chamber, such as a dish, tray, or channel that provides a common lower chamber for ion transport measuring means that connect to separate upper chambers. In one embodiment, at least a portion of a lower chamber piece can be in the form of a gasket that seals around the bottom of the biochip that when sealed against a lower chamber base piece or platform provides an inner space as a lower chamber Alternatively, the device can be assembled with a lower chamber piece that comprises at least a portion of more than one lower chamber. In this case, each individual lower chamber preferably connects with a single upper chamber via an ion transport measuring hole in the biochip. The lower chamber piece can form the walls and lower surfaces of lower chambers, or the lower chamber piece can form at least a portion of the walls of a lower chamber and other parts can form the bottom surface of the lower chambers. In one embodiment, at least a portion of a lower chamber piece can be in the form of a gasket that seals around the bottom of the biochip and having openings such that when the gasket is sealed against a lower chamber base piece or platform the inner spaces of the gasket openings provide lower chambers.

A lower chamber piece can be irreversibly attached to a cartridge of the present invention, such as by the use of adhesives, but preferably, a lower chamber piece is reversibly attached to a cartridge. Reversible attachment can be by any feasible means that provides a fluid-impermeable seal between the walls of the lower chamber or chambers and the lower surface of the chip, such as pressure mounting, and can use clamps, frames, screws, snaps, etc.

In one example of attachment of a lower chamber piece to a cartridge, a lower chamber piece structure comprising a compressible material such as PDMS contains channels for fluid delivery and other channels for applying vacuum pressure that can maintain a strong seal between the biochip and the structure, where the vacuum pressure provides the means of reversible attachment of the lower chamber piece to the biochip. Preferably, the applied vacuum pressure also scavenges any leaks that may occur or develop between lower chambers that would otherwise result in electrical cross-talk between adjacent lower chambers.

Preferred embodiments encompass devices that comprise multiple ion transport measuring units, comprising an upper chamber piece that comprises at least two upper chambers that are open at both their upper and lower ends and a chip that comprises at least two ion transport measuring means in the form of holes through the chip that are in register with the upper chambers. The upper chamber piece and chip can be reversibly or irreversibly attached to a lower chamber piece that comprises at least a portion of at least two lower chambers that are in register with the ion transport measuring means and upper chambers. Such preferred devices comprise multiple ion transport measuring units, where each unit comprises an upper chamber and a lower chamber, each in register with a hole in the biochip, in which the hole connects the upper with a lower chamber. The interaction between the chambers and the chip are such that at least one of the chambers of an ion transport measuring unit can be pneumatically sealed and can withstand pressures of at least plus or minus 100 mmHg, and preferably at least plus or minus 1 atmosphere of pressure.

In some preferred aspects of the present invention, a cartridge comprises an upper chamber piece comprising multiple upper chambers irreversibly attached to a chip comprising multiple ion transport measuring holes that can be reversibly engaged with a lower chamber piece that comprises at least a portion of multiple lower wells, such that the upper wells and lower wells of the device are in register with one another and with the ion transport measuring holes of the chip.

Preferred devices and apparatuses for ion transport measurement can have two or more, four or more, eight or more, or sixteen or more ion transport measuring units. For example, ion transport measuring devices can have sixteen, twenty-four, forty-eight, or ninety-six or more ion transport measuring units.

Lower chamber pieces that comprise at least a portion of multiple lower chambers of a multiple unit ion transport measuring apparatus can be provided in a variety of designs. Lower chamber pieces can comprise complete lower chamber units, or can comprise all or a portion of the walls of the multiple chamber units, such that when the lower chamber piece is fixed to or pressed against the lower side of a biochip and attached to or pressed down on a platform or lower chamber base piece, the lower chamber piece forms the walls and the platform or lower chamber base piece forms the bottoms of the lower chambers.

For example, a device for measuring ion transport function or activity can comprise a multiple unit device that comprises an upper chamber piece having multiple upper chambers in the form of wells that are open at both the top and bottom, and a chip attached to the upper chamber piece, where the chip comprises multiple holes for ion transport measurement that are spaced such that when the device is assembled each upper chamber is over a hole. A lower chamber piece can be held or fastened against the lower side of the chip of the device, where the lower chamber piece comprises multiple openings that fit over the biochip holes to form lower chambers.

In a preferred embodiment, the lower chamber piece comprises at least one compressible plastic or polymer on its upper surface that can form a fluid-impermeable seal with the bottom of the biochip. The lower chamber piece can also comprise at least one compressible polymer as a gasket on its lower surface that can form a seal with a platform or a lower base piece. In this design, when the device is positioned on a lower base piece or platform so that the lower chamber piece is pressed against the lower base piece or platform, the lower base piece or platform forms the bottom of the lower chambers. Mechanical pressure can provide a seal between the biochip and the lower chamber piece, and between the lower chamber piece and the platform. Clamps can optionally be employed to hold the seal. The compressible plastic or polymer can comprise rubber, a plastic, or an elastomer, such as for example, polydimethylsiloxane (PDMS), silicon polyether urethane, polyester elastomer, polyether ester elastomer, olefinic elastomer, polyurethane elastomer, polyether block amide, or styrenic elastomer. Preferably, in cases where the compressible plastic or polymer contacts cells, the compressible plastic or polymer is made of a biocompatible material, such as PDMS. Portions of the lower chamber piece that do not form a gasket can be of any suitable material, including plastics, waxes, polymers, glass, metals, and ceramics. Portions of the lower chamber piece that contact measuring solutions preferably comprise materials that are not affected by electrical current (such as nonmetals).

For example, one preferred design of a device for ion transport measurement comprises an upper chamber piece, a chip comprising ion transport measuring holes, a lower chamber piece, and a lower base piece in the form of a platform. The chip has been chemically treated, preferably with at least one base, to enhance its sealing properties. The lower chambers that are formed by a lower chamber piece that comprises an aluminum frame having a PDMS gasket on its upper surface that fits over the lower surface of a chip. PDMS is also used to coat the inner surfaces of the holes that form the lower chambers, and is also used as a gasket on the bottom of the lower chamber piece. The lower chambers can be filled with a solution while the device is held in inverted orientation prior to positioning the device on the platform. During use of the device, mechanical pressure holds the lower chamber piece against the chip and against the platform.

The lower base piece can optionally comprise one or more electrodes. For example, separate individual electrodes can be fabricated on or attached to the platform so that separate lower chambers of the device have independent electrodes that can be attached to independent circuits and used as patch clamp recording electrodes. In an alternative design, the platform can comprise or be part of a common lower chamber with a reference electrode, or a common electrode that can be used as a reference electrode can contact all of the lower chambers of a device having multiple lower chambers (optionally through separate electrode extensions that meet a common connector outside of the chambers).

The lower base piece can optionally comprise or engage one or more conduits connected to tubing that can allow for the flow of fluids into and out of individual lower chambers. In preferred embodiments, a device of the present invention comprises one or more flow-through lower chambers where each of the one or more lower chamber connects to at least one conduit for providing solutions to the lower chamber (the inflow conduit) and at least one additional conduit for removing solutions from the lower chamber (the outflow conduit).

FIG. 2 depicts a single ion transport measuring unit of a device in which a gasket (24) forms the walls of the lower chamber (25). The upper well (21) is part of an upper chamber piece that is attached to a chip (23) having an ion transport measuring means in the form of a hole (22). An inflow conduit (27) and outflow conduit (28) connects to each lower chamber. In this type of design the lower chambers can be filled with a measuring solution (such as an intracellular solution) after the gasket is positioned on a lower base piece. The conduits can also be used for the exchange of solutions during the use of the device. For example, solutions containing test compounds, ionophores, inhibitors, drugs, different concentrations or combinations of ions or compounds, etc., can be delivered into and out of a chamber during ion transport measuring assays. At least some of the conduits or tubing can optionally comprise or lead to electrodes (such as, for example, recording electrodes). In the design depicted in FIG. 2, a lower chamber electrode (26) is situated on, fabricated on, or attached to the lower chamber piece.

The present invention also includes methods of using an ion transport measuring device of the present invention that comprises at least one upper chamber piece reversibly or irreversibly attached to a chip, wherein the chip comprises at least one ion transport measuring means in the form of a hole through the biochip, wherein the chip has been treated to have enhanced electrical sealing properties. The device further comprises at least one lower chamber, wherein at least one well of the upper chamber piece comprises, contacts, or is in electrical contact with at least one electrode, and the at least one lower chamber In one preferred design, a lower chamber piece comprises conduits that engage each lower chamber from one side (one per chamber), and conduits that engage each lower chamber from the opposite side. Conduits on one side of the lower chamber piece can be used for introducing solutions, such as “intracellular solutions” that can optionally comprise test compounds, into the chambers, and conduits on the opposite side of the lower chamber piece can be used for flushing solutions and/or air bubbles out of the lower chambers. At least one set of the conduits (such as, for example, the inflow conduits) can comprise wire electrodes that are independently connected (with respect to other ion transport measuring units) to a signal amplifier and used for ion transport activity recording.

Devices such as those described herein can be part of apparatuses that also comprise patch clamp signal amplifiers and conduits, fluid dispensing means, pumps, electrodes, or other components. The apparatuses are preferably mechanized, for automated fluid dispensing or pumping, pressure generation for sealing of particles, and ion transport recording. The apparatuses can be part of a biochip system for ion transport measurement, in which software controls the automated functions.

The present invention also includes methods of using an ion transport measuring device of the present invention to measure one or more ion transport properties or activities of a cell or particle (such as, for example, a membrane vesicle). The methods include using a device that comprises at least one upper chamber reversibly or irreversibly attached to a chip that comprises at least one ion transport measuring means in the form of a hole through the biochip, wherein the chip has been treated to have enhanced sealing properties. In the assembled device used in the methods of the present invention, the holes of the biochip access at least one lower chamber. In these methods, the device is reversibly or irreversibly attached to a lower chamber piece that forms all or a portion of a lower chamber. An upper chamber piece and chip can optionally additionally be reversibly or irreversibly attached to a platform or lower chamber base piece that can form at least the lower surface of one or more lower chambers. For example, a cartridge comprising an upper chamber piece and chip can be attached to at least one lower chamber piece that forms the walls and lower surfaces of one or more lower chambers, or a cartridge can be attached to at least one lower chamber piece that forms the walls of one or more lower chambers and at least one platform or lower chamber base piece that forms the lower surfaces of one or more lower chambers.

The device is assembled such that the one or more upper chambers are in register with the one or more ion transport measuring holes of the chip, and one or more lower chambers access the one or more upper chambers via the one or more holes of the chip. In preferred embodiments, each of the one or more upper chambers is in register with one of the ion transport measuring holes of the chip, and each of the lower chambers is aligned with one upper chamber that it accesses via an ion transport measuring hole.

During use of the device, the one or more upper chambers comprise, contact, or are in electrical contact with at least one electrode. During use of the device, the one or more lower chambers comprise, contact, or are in electrical contact with at least one electrode. In one alternative, the one or more upper chambers contact, comprise, or are in electrical contact with a common reference electrode, and the one or more lower chambers contact, comprise, or are in electrical contact with a individual reference electrodes. In another alternative, the one or more upper chambers contact, comprise, or are in electrical contact with individual reference electrodes, and the one or more lower chambers contact, comprise, or are in electrical contact with a common reference electrode.

The method includes: filling at least one lower chamber of the device with a measuring solution; adding at least one cell or particle to one or more of the at least upper chambers of the device, wherein the one or more upper chambers is connected to one of the at least one lower chambers that comprises measuring solution via a hole in the ion transport measuring chip; applying pressure to at least one lower chamber, at least one lower chamber, or to an upper chamber and a lower chamber that are connected via an ion transport measuring hole to create a high-resistance electrical seal between at least one cell or particle and at least one hole; and measuring at least one ion transport property or activity of the at least one cell or at least one particle.

Preferably, one or more cells or one or more particles are in a suspension when added to the upper chamber. Various measuring solutions and, optionally, compounds can be provided in an upper chamber or a lower chamber.

In some preferred embodiments, the methods measure at least one ion transport activity or property of a cell in the whole cell configuration, but this is not a requirement of the present invention, as the devices can be used in a variety of applications on particles such as, for example, vesicles, as well as cells.

The application of pressure can be manual or automated. If pressure is applied manually (for example, by means of a syringe), preferably the user can make use of a pressure display system to monitor the applied pressure. Automated application of pressure can be through the use of a software program that is able to receive feedback from the device and direct and control the amount of pressure applied to one or more ion transport measuring units.

Various specific ion transport assay can be used for determining ion transport function or properties. These include methods known in the art such as but not limited to patch clamp recording, whole cell recording, perforated patch whole cell recording, vesicle recording, outside out or inside out recording, single channel recording, artificial membrane channel recording, voltage-gated ion transport recording, ligand-gated ion transport recording, recording of energy requiring ion transports (such as ATP), non energy requiring transporters, toxins such a scorpion toxins, viruses, stretch-gated ion transports, and the like. See, generally Neher and Sakman, Scientific American 266:44-51 (1992); Sakman and Neher, Ann. Rev. Physiol. 46:455-472 (1984); Cahalan and Neher, Methods in Enzymology 207:3-14 (1992); Levis and Rae, Methods in Enzymology 207:14-66 (1992); Armstrong and Gilly, Methods in Enzymology 207:100-122 (1992); Heinmann and Conti, Methods in Enzymology 207:131-148 (1992); Bean, Methods in Enzymology 207:181-193 (1992); Leim et al., Neurosurgery 36:382-392 (1995); Lester, Ann. Rev. Physiol 53:477-496 (1991); Hamill and McBride, Ann. Rev. Physiol 59:621-631 (1997); Bustamante and Varranda, Brazilian Journal 31:333-354 (1998); Martinez-Pardon and Ferrus, Current Topics in Developmental Biol. 36:303-312 (1998); Herness, Physiology and Behavior 69:17-27 (2000); U.S. Pat. No. 6,117,291; U.S. Pat. No. 6,107,066; U.S. Pat. No. 5,840,041 and U.S. Pat. No. 5,661,035; Boulton et al., Patch-Clamp Applications and Protocols, Neuromethods V. 26 (1995), Humana Press, New Jersey; Ashcroft, Ion Channels and Disease, Cannelopathies, Academic Press, San Diego (2000); Sakman and Neher, Single Channel Recording, second edition, Plenuim Press, New York (1995) and Soria and Cena, Ion Channel Pharmacology, Oxford University Press, New York (1998), each of which is incorporated by reference herein in their entirety.

II. An Ion Channel Measurement Device Having Flow-Through Lower Chambers

The present invention includes ion transport measurement devices and apparatuses comprising flow-through lower chambers. As used herein, a “flow-through chamber” is a chamber to which fluids can be added and from which fluids can be removed via continuous fluid flow. Thus, a flow-through chamber will preferably engage at least two conduits: at least one inflow conduit for adding fluids (such as solutions) and at least one outflow conduit for the removal of fluids (such as solutions). In the alternative, a flow-through chamber can be designed as a channel through which fluids can pass.

A flow-through lower chamber can be designed with two or more ports or openings in the wall of the chamber, such that at least one inflow conduit and at least one outflow conduit engage one or more walls of the lower chamber at the ports. In an alternative, at least one inflow conduit and at least one outflow conduit can engage ports or openings at the bottom surface of a chamber. It is also possible to have a flow-through chamber in which at least one conduit engages the wall of the chamber and at least one conduit engages the bottom surface of the chamber.

Flow-through lower chambers have several advantages for ion transport measuring devices. Because the exchange of lower chamber solutions can be performed rapidly and continuously, without the need to empty the chamber of liquid when changing from a first solution to a second solution, a single patch clamp (that is, a cell or particle sealed with a high resistance electrical seal to an ion transport measuring hole) can be used for repeated tests, using, for example, different solutions that are delivered to the chamber in sequence. Adding and removing solutions in a flow-manner via conduits also facilitates automation of an ion transport measurement device, where the addition and removal of solutions can be through the automated control of pumps and valves. Addition or removal of solutions to one or more lower chambers can preferably but optionally be performed independently of the fluid distribution to other chambers of a device, so that conditions of particular patch clamps can be changed without disrupting or changing the conditions of other patch clamps of the device.

In preferred embodiments, an ion transport measurement device comprises one or more flow-through lower chambers, at least one chip comprising ion transport measuring holes, and at least one upper chamber. Preferably, a flow-through chamber is connected to two or more conduits that can provide fluid flow to and from a lower chamber. At least one of the at least two conduits can be used to provide solutions to a lower chamber, and at least one other of the at least two conduits can be used to remove solutions from a lower chamber.

Preferably, fluid flow is directed by one or more fluid pressure sources such as a pump or pumps. The conduits, or tubing or connectors leading to the conduits, can comprise valves that can be used to control the flow of solutions into or out of a lower chamber. In some preferred embodiments, control of the flow of solutions into or out of a chamber is automated, at least in part.

Lower chambers can be formed by one or more pieces of the device. At least a portion of the upper surface of a lower chamber will be formed by a chip comprising an ion transport measuring hole. The walls and bottom surface of a lower chamber can be formed by one or more pieces of the device. For example, in some embodiments at least a portion of the walls and the bottom surface of a lower chamber can be formed by a lower chamber piece. In other preferred embodiments, at least a portion of the walls of a lower chamber can be formed by a lower chamber piece and the bottom surface of a lower chamber can be formed by a lower chamber base piece or a platform.

In some embodiments, an ion transport measuring device with one or more flow-through lower chambers can comprise a lower chamber piece that has inflow and outflow conduits that directly or indirectly connect to the walls or bottom surfaces of the one or more lower chambers. In some designs, the device can comprise a platform or a lower chamber base piece that comprises inflow and outflow conduits that directly or indirectly connect to the bottom surface of one or more lower chambers. In an especially preferred embodiment of the present invention, a device for ion transport measurement comprises a lower chamber base piece that forms the bottom of multiple lower chambers and comprises conduits that open to the lower surfaces of the lower chambers, such that each lower chamber is accessed by an inflow conduit and an outflow conduit. In this design, the device further comprises a lower chamber piece that forms at least a portion of the lower chamber walls, a chip comprising ion transport measuring holes that align with the lower chambers, and an upper chamber piece that comprises multiple upper wells that align with the ion transport measuring holes of the chip and the lower chambers formed by the lower chamber piece and lower chamber base piece.

In preferred embodiments of ion transport measuring devices having one or more flow-through lower chambers, the devices have multiple flow-through lower chambers, each of which engages an inflow conduit and an outflow conduit, such that inflow and outflow conduits connected to different chambers are separate and independent.

Components of an ion transport measuring device having one or more flow-through lower chambers, such as a lower chamber base piece, lower chamber piece, chip, and an upper chamber piece, can be reversibly or irreversibly attached to one another. In some preferred embodiments, an upper chamber piece and chip are irreversibly attached (such as by adhesives) to one another as a cartridge, and the cartridge can be reversibly attached to a lower chamber piece and lower chamber base piece. A cartridge can be attached to a lower chamber piece by any feasible means that provides a fluid impermeable seal between the lower surface of the chip of the cartridge and the walls of the one or more lower chambers that are formed, at least in part, by a lower chamber piece. In designs in which the device comprises a lower chamber base piece, the lower chamber base piece can be attached to a lower chamber piece by any feasible means that provides a fluid impermeable seal between the lower chamber piece and the lower chamber base piece. The attachment of a lower chamber base piece to a lower chamber base can be irreversible, but is preferably reversible. For example, reversible attachment can be by pressure mounting, and can use compressible materials as well as clamps, frames, screws, snaps, etc.

In preferred embodiments encompassing devices having more than one ion transport measuring unit, when a multiunit device is assembled, the two or more wells of the upper chamber piece are in register with the two or more holes of the biochip, and the two or more lower chambers formed by a lower chamber piece and lower chamber base piece are aligned with the holes with the biochip. The lower chamber base piece comprises at least two inflow conduits and at least two outflow conduits, such that each lower chamber is accessed by an inflow conduit and an outflow conduit.

In some preferred embodiments, a cartridge, lower chamber piece that comprises a compressible material and a lower chamber base piece are fastened together using a clamp. In other preferred embodiments, a cartridge, lower chamber piece, and, optionally, a lower chamber base piece are attached using pressure mounting and at least one gasket to form seals between the parts.

The present invention also includes a lower chamber base piece for use in a device for ion transport measurement that can optionally be used independently of a larger automated apparatus and can be used to observe cells and particles within the device using an inverted microscope. In this embodiment, at least a portion of the lower chamber base piece that will form the bottom surface of the lower chambers is transparent. Preferably, the lower chamber base piece comprises at least two conduits that extend through the lower chamber base piece such that when the lower chamber base piece is assembled into a device of the present invention, the conduits can be used to transfer fluid from outside the device into lower chambers, and transfer fluid from inside lower chambers to the outside of the device. As part of a device for ion transport measurement, the base piece forms a bottom surface of lower chambers. The conduits that extend through the base piece allow for fluids such as solutions to be delivered in and out of lower chambers of ion transport measuring devices.

In this embodiment, two or more conduits go through the base piece, with each conduit having one opening on one surface of the base piece, and the other opening on a different surface of the base piece. In preferred embodiments of the present invention, the conduits extend from a side of the base piece essentially horizontally toward the center, and then turn or curve upward to end in an opening on the top surface of the base piece which, in an assembled device, is the bottom surface of a lower chamber. The side opening can be the site where the conduit connects with tubing connected to solution reservoirs, pressure sources, and/or electrodes, and the top opening of the conduits is the site where the conduit opens into a lower chamber. In a preferred device of the present invention, each lower chamber of an ion transport measuring device is connected to two such conduits, and the conduits can provide for solutions to be delivered into and washed out of a lower chamber.

A lower chamber piece and lower chamber base piece can comprise one or more plastics, one or more polymers, one or more ceramics, silicon or glass. Preferably, the part or parts of a lower chamber base piece that will form the bottom of one or more lower chambers of an ion transport measuring device is preferably made of a transparent material that is impermeable to aqueous liquids so that cells or particles inside an ion transport measuring unit are visible using an inverted microscope. Although not a requirement of the present invention, to simplify manufacture of the base piece, the entire base piece (with the exception of separate attachments such as connectors, pins, screws, etc.) is preferably made of a single material by molding or machining. Glass and transparent polymers are preferred materials, with transparent polymers such as polycarbonate and polystyrene having the advantage of easier manufacture.

Conduits can be molded into or drilled through the base piece, and can be fitted with connectors. (Connectors can comprise glass, polymers, plastics, ceramics, or metals.) The connectors can be connected to tubing that can be used to provide in-flow and outflow of solutions to a lower chamber of an ion transport measuring unit.

The conduits can also be used to deliver pressure to the lower chamber and to an ion transport measuring hole of a chip exposed to the chamber. Pressure can be generated, for example, by a pump or a pressure source connected to the tubing that will be filled with an appropriate solution in at least the segment connecting the lower chamber. Preferably the pressure is regulatable and can be used for purging air bubbles and or other blocking micro-particles in the ion transport measuring hole, cell and particle positioning, sealing, and optionally, membrane rupture of an attached cell when carrying out ion transport measurement procedures.

In preferred embodiments, the conduits, or tubes leading to the conduits, can also comprise electrodes. For example, a wire electrode can be threaded through tubing that is connected to a conduit of a base piece. The wire electrode can optionally extend through the conduit to the upper surface of the base piece (which will be the lower surface of a lower chamber of an ion transport measuring unit).

In the alternative, the base piece can comprise one or more electrodes on its upper surface. Electrodes fabricated or attached to the upper surface of the base piece can be connected through leads to connectors on the outer edge of the base piece, and the connectors can be connected to a patch clamp amplifier.

In preferred aspects of the present invention, a lower chamber base piece is designed to form the bottom of more than one lower chamber of an ion transport measuring device. Preferably, a lower chamber base piece is designed to form the bottoms of all the lower chambers of an ion transport measuring device that comprises at least two ion transport measuring units, more preferably at least six ion transport measuring units, and more preferably yet, at least sixteen ion transport measuring units. In a preferred embodiment described in detail in Example 5, a lower chamber base piece forms the bottom of 16 lower chambers of a 16 unit device. In many cases (as illustrated in the example) multiple lower chambers will be arranged linearly in a row, but this is not a requirement of the present invention.

Thus, in preferred embodiments of the present invention, a flow-through lower chamber base piece will comprise multiple conduits, two for each lower chamber that will occur in the ion transport measuring device: a first conduit for inflow of solutions (the “inflow conduit”), and a second conduit for outflow of solutions (the “outflow conduit”). A schematic cross-sectional view of a single ion transport measuring unit of one design of a device of the present invention having one or more flow-through lower chambers is shown in FIG. 2. In this depiction, the lower chamber (25) is accessed by an inflow conduit (27) and an outflow conduit (28). In this depiction the lower chamber comprises an electrode (26) positioned on the upper surface of the lower chamber base piece. In an alternative design, one of each pair of conduits that leads to a single chamber of an ion transport measuring device can contain or contact an electrode.

The present invention also includes devices and apparatuses for ion transport measurement that include a lower chamber base piece of the present invention. In one embodiment of the present invention, a device includes: a lower chamber base piece that comprises at least two conduits, where at least a portion of the lower chamber base piece is transparent; a chip comprising at least one ion transport measuring hole; and an upper chamber piece that comprises at least one chamber that attaches to said chip. Preferably, the device also includes a lower chamber piece in the form of at least one gasket that fits between the lower chamber base piece and the chip where the one or more gaskets comprise at least one opening, such that the one or more gaskets form the walls of the one or more lower chambers and seals the lower chamber base piece to the chip. The gasket or gaskets align with the lower surface of the chip such that a lower chamber formed by a gasket comprises a lower surface having the openings of two conduits, and an upper surface comprising a portion of a chip having a single ion transport measuring hole.

In preferred aspects of the present invention, a lower chamber base piece is designed to fit a base plate that is adapted to fit the stage of a microscope, such as an inverted light microscope. The dimensions can be altered to fit a microscope of choice, such as, for example, an inverted light microscope sold by Leica, Nikon, Olympus, Zeiss, or other companies.

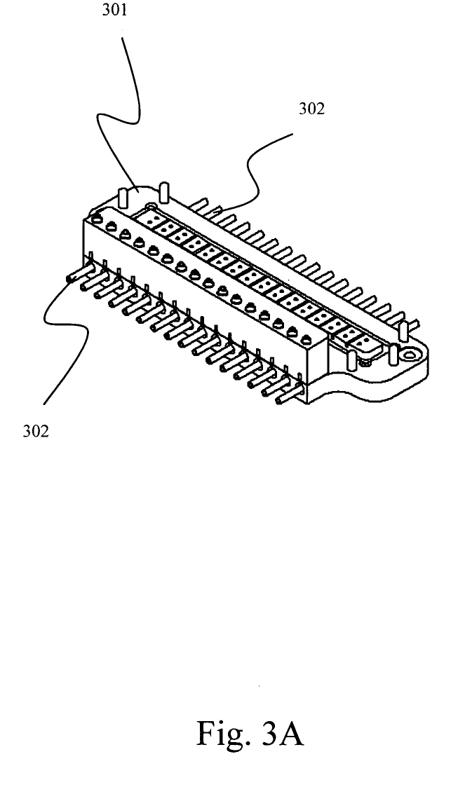

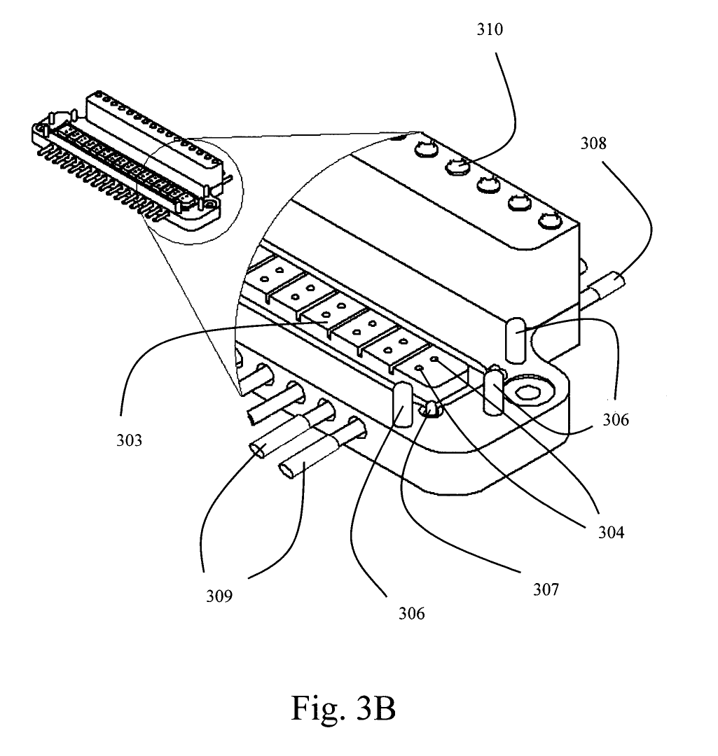

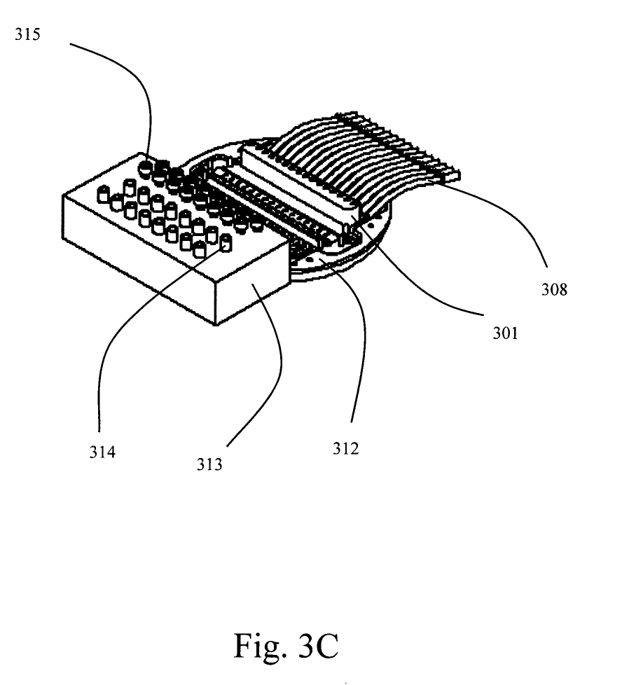

FIG. 3A provides a photograph of a preferred design of a lower chamber base piece having flow-through chambers for use in a sixteen unit device. In FIG. 3(A), connectors (302) for inflow conduits can be seen leading out from one side of the lower chamber base piece (301) and connectors (302) for outflow conduits can be seen leading out of the opposite side of the lower chamber base piece. FIG. 3(B) is a close-up photograph of the lower chamber piece showing the areas that correspond to what will be the transparent bottom surfaces (303) of the lower chambers when the device is fully assembled (black areas) with the conduit openings (304) visible as lighter areas within the black areas. A transparent gasket (305) lies over the top of the central portion of the lower chamber piece covering the areas that will be the bottom surfaces of the lower chambers (303). In this design, the gasket can be aligned over the lower chamber base piece by fitting a ridge that runs lengthwise down the underside of the gasket into a groove the runs lengthwise down the length of the upper surface lower chamber base piece. The gasket depicted has two ridges running along either side of the gasket (on either side of the row of openings) and the lower chamber base piece has two corresponding grooves on either side of the surface having conduit openings (not visible in the photographs). When the gasket is placed on the lower chamber base piece such that the ridges of the gasket fit the grooves of the lower chamber base piece, the openings of the gasket align over the areas of the surface of the lower chamber base piece that have conduit openings and will be the bottom surfaces of the lower chambers.

The lower chamber base piece can also have “cuts” between the areas that will correspond to the bottom surface of lower chambers (the cuts are perpendicular to the alignment grooves, not visible in the photographs). When the gasket is placed on top of the lower chamber base piece, the cuts in the lower chamber base piece are between lower chamber areas defined by the openings in the gasket. These cuts can reduce the possibility of solution seepage between lower chambers.

The three alignment dowels (306) seen in the foreground of FIG. 3B at lower left are used to align an upper chamber piece or cartridge over the lower chamber base piece, such that the ends of the lower chamber base piece fit between and abut the three pins. The two shorter pogo pins (307) are used to prevent a clamp placed on an assembly that includes a cartridge (comprising an upper chamber piece and attached chip) a gasket, and a lower chamber base piece from pressing down on the assembly prior to fastening of the clamp. Holding the clamp in standoff position by these pogo pins (307) prior to fastening prevents misaligned contact of the cartridge with the gasket.

Also seen in FIG. 3B, are inflow tubes (309) and outflow tubes (308) attached to the connectors in this view. Female pin sockets (310) that connect to the lower chamber recording electrodes can also be seen. Electrical connectors that are attached to a signal amplifier can be inserted into these socket pins.

In FIG. 3C, the lower chamber base piece is seated in a base plate (312) adapted to a microscope stage. To the right of the base plate is a plexiglass piece (313) comprising ports (314) for the addition of lower chamber solutions and screw-down pinch valves (315) for the inflow tubing.

A baseplate can be made of any suitable material, such as glass, plastics, polymers, ceramics, or metals. Metals, such as but not limited to stainless steel, are preferred, because metal materials have high mechanical strength needed during pressure sealing of the lower chamber. A metal base plate can also, together with a grounded microscope stage, form an electrical noise shield around a lower chamber piece fitted to the base plate.

The base plate can be carved on the top side to catch any fluids that may leak or spill and prevent the contamination of the microscope with the fluids. Preferably, the base plate is sealed around the lower chamber base piece, for example, with silicone glue, silicone grease, Vaseline, etc.

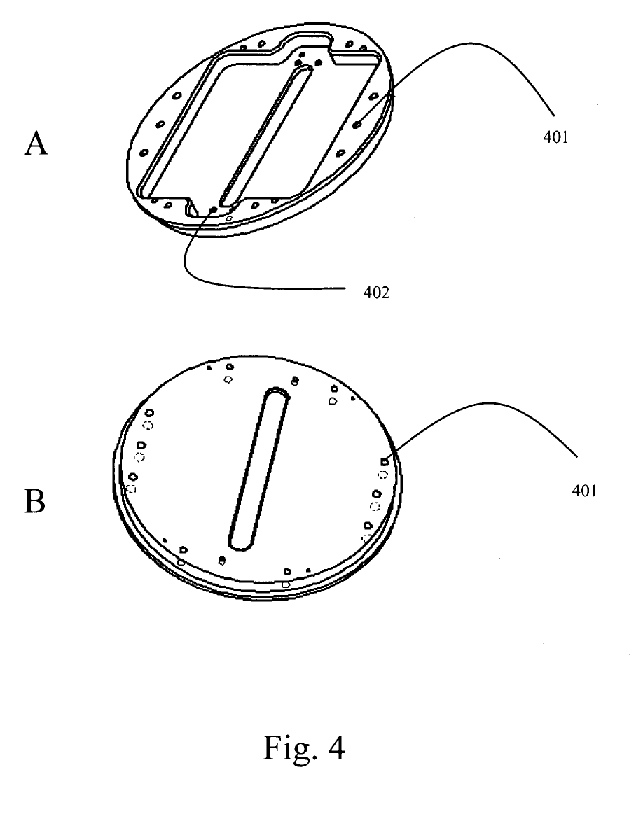

The base plate is preferably drilled and tapped so as to provide a mounting point for the lower chamber base piece and for a clamp that can hold additional components of the ion transport measuring device together (for example, gasket, chip, upper chamber piece) to form the upper and lower chambers of ion transport measuring units. The base plate is designed to hold an ion transport measuring device within a few millimeters of the level of the top of the microscope stage so as to ensure that the chip function may be monitored within the focal range of the microscope. FIG. 4 illustrates the design of a base plate as adapted for a Nikon Microscope.

Flow-through lower chamber designs described herein can be used in ion transport measurement devices of the present invention. In preferred embodiments, such devices comprise upper chamber pieces having multiple wells and chip comprising multiple ion transport measuring holes. Upper chambers of such devices can comprise one or more electrodes. Such electrodes can be fabricated, positioned, or attached on a surface of an upper chamber, such as those described in a later section of this application on two-piece molding of upper chambers, can be inserted into the upper chambers of the assembled device from above (for example, wire electrodes inserted into the wells), or can be provided as within a tube or part of a tube that can be placed inside the upper chamber (such as a tube that delivers solutions or cell suspensions). Preferably, electrodes of upper chambers are connected as a common reference electrode, but this is not a requirement of the present invention. It is also possible for each upper well to have an individual (recording) electrode, and to have the electrodes of the lower chambers connected as a common reference electrode.

In some preferred embodiments, the upper piece of a device of the present invention comprises a common reference electrode that contacts all of the wells. In other preferred embodiments, an electrode is not within or attached to the upper piece, but during assembly of the device is inserted into an upper well through upper opening of the well. In other preferred embodiments, an electrode can be brought into electrical contact with an upper chamber by way of a conduit that comprises an electrode or can provide an electrolyte solution bridge to an electrode. Electrodes that are connected through electrolyte bridges can be recording electrodes, but in most preferred embodiments are reference electrodes.

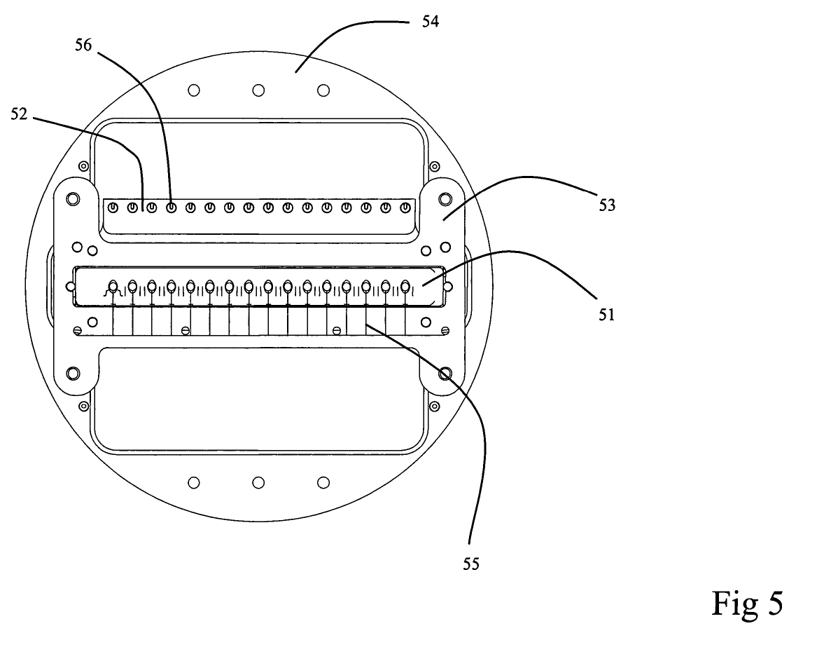

FIG. 5 depicts the design of a device of the present invention having an upper chamber piece (51) and attached chip (not visible beneath the upper chamber piece) fixed on top of a gasket (not visible beneath the upper chamber piece) and lower chamber base piece (not visible beneath the upper chamber piece) by means of a clamp (53). The clamp (53) also fixes the device to a baseplate (54) adapted to a microscope. The plexiglass piece (52) holds female pin sockets (56) that connect to electrodes inserted into lower chamber piece conduits. The clamp has a wire electrode (55) that extends into upper chamber wells.

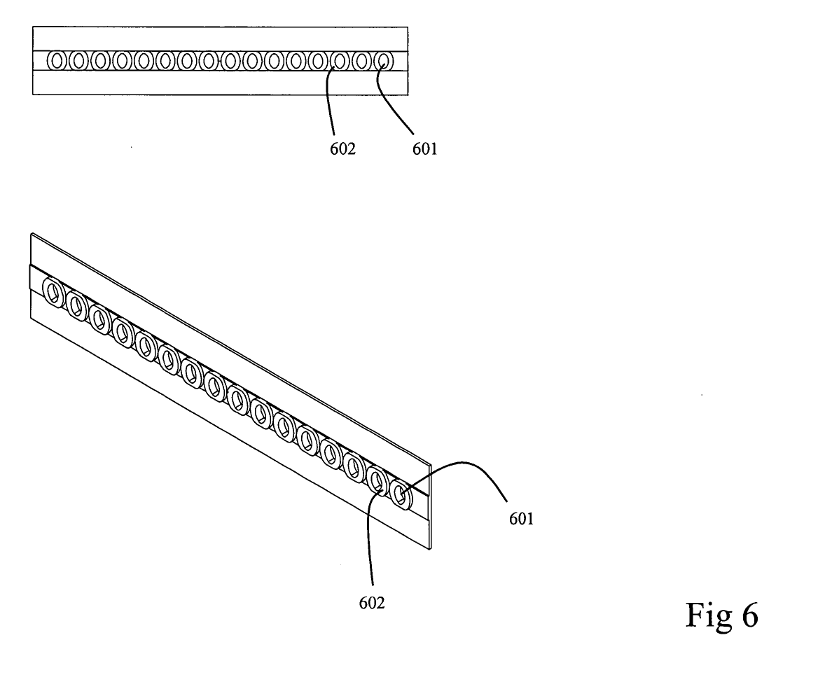

FIG. 6 shows a gasket that can fit on top of a lower chamber base piece and form the walls of lower chambers such that the openings (601) in the gasket become the lower chamber spaces.

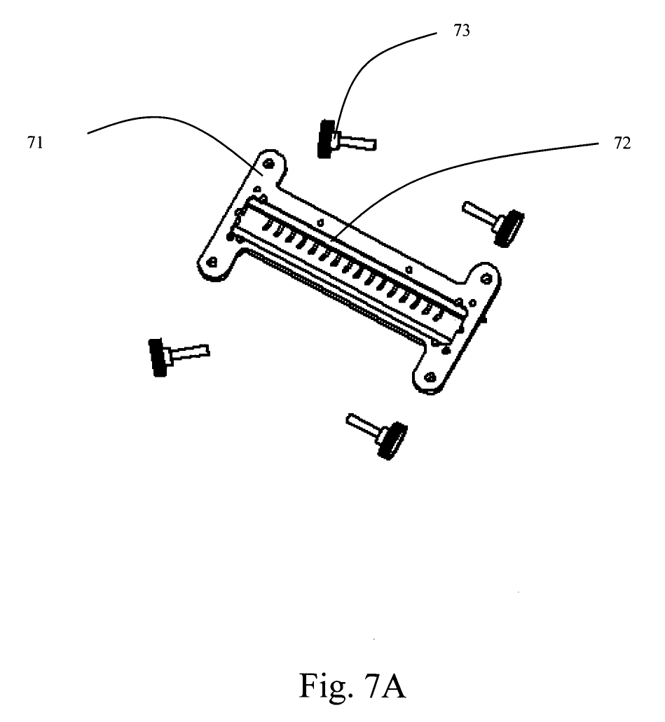

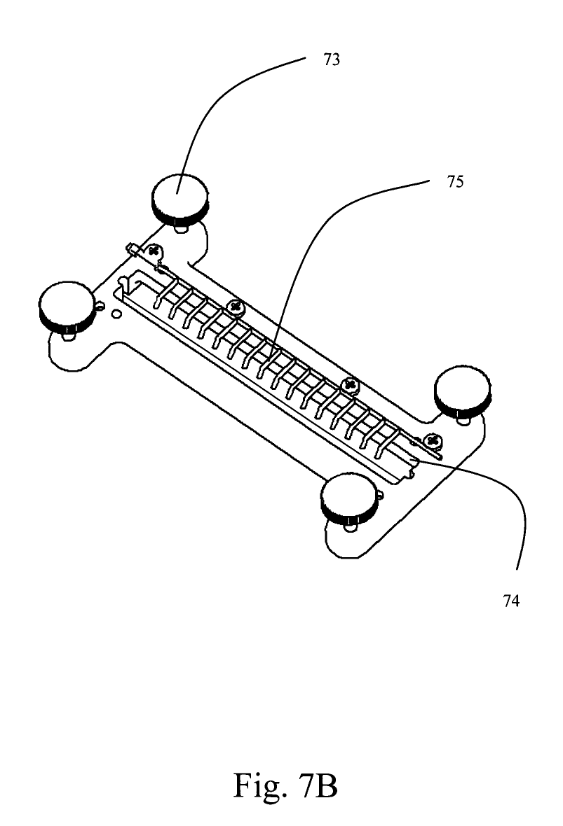

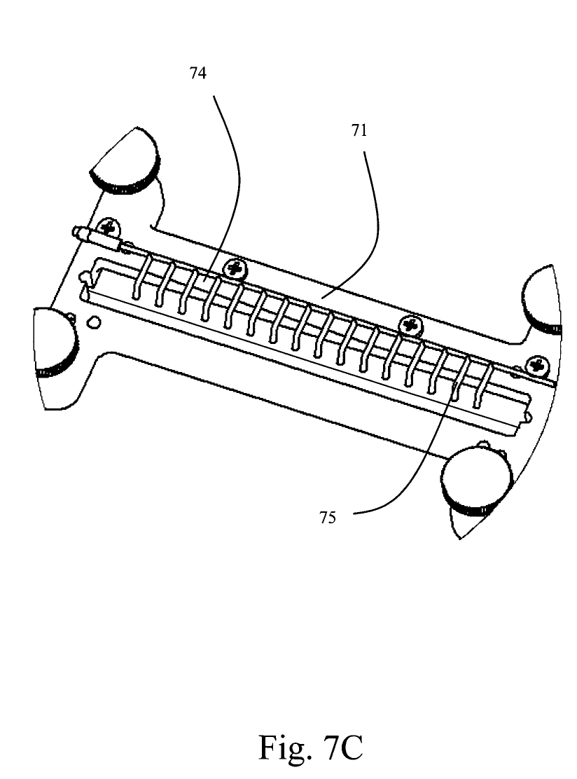

FIG. 7 provides three views of one design of a clamp that can be used in the assembly of a device of the present invention. In FIG. 7A, the clamp (71) is shown upside down to illustrate the cutout (72) that fits a cartridge. Thumb screws (73) used to attach the clamp to the base piece are alongside the clamp (71). In FIG. 7B, the top view of the clamp on the cartridge (74) reveals the presence of an array of top chamber electrodes (75) that reach into the cartridge wells.

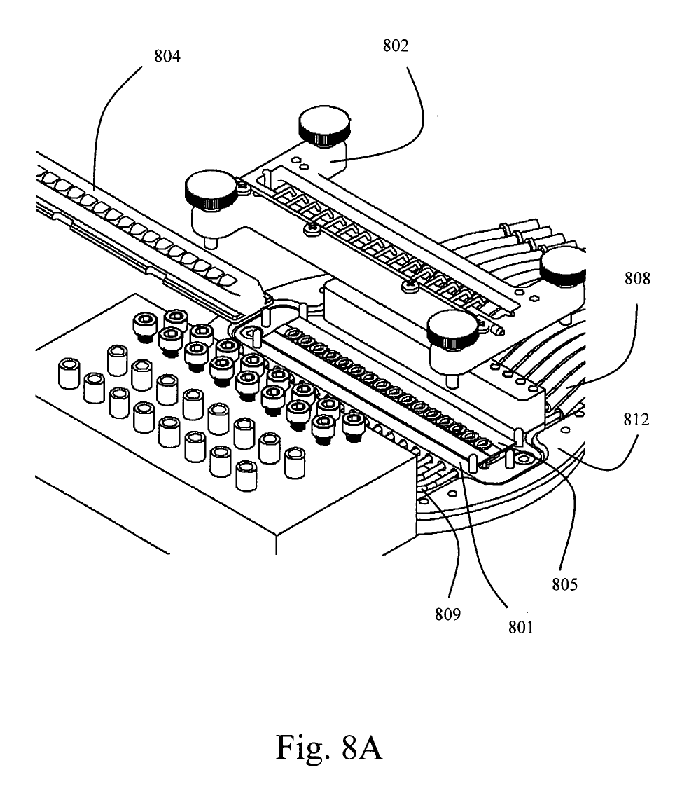

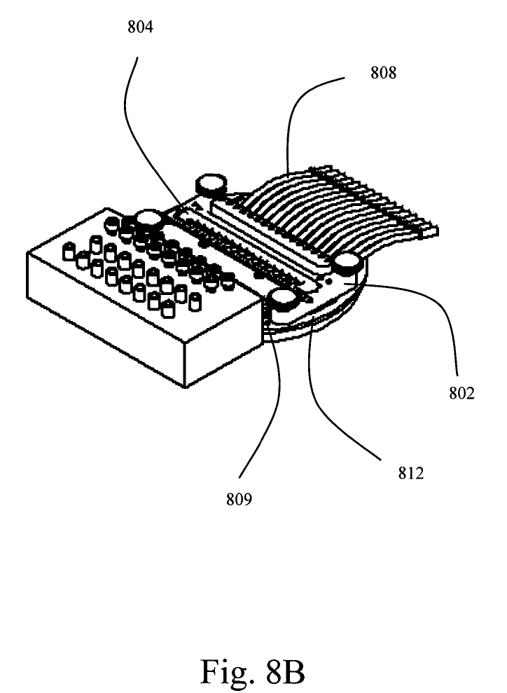

FIG. 8 provides photographs showing the parts of an ion transport measuring device of the present invention including a baseplate (812), a cartridge (804) comprising an upper chamber piece with a chip attached at the bottom, lower chamber base piece (801), and clamp. In FIG. 8A, the black upper chamber piece of the cartridge (804), transparent lower chamber base piece (801), inflow tubing (809) and outflow tubing (808) leading to the lower chamber base piece (801), and metallic clamp (802) can be seen. The transparent gasket (805) is lying over the lower chamber base piece (801) behind the upper chamber cartridge. In FIG. 8B, the device is assembled, with the clamp (802) screwed into a baseplate (812).

The present invention also encompasses compositions and devices that incorporate novel elements of the compositions and devices described herein, including: a transparent platform beneath the lower chambers, a baseplate adapted for microscope stage, one or more flow-through bottom chambers, reference or recording electrodes outside of upper or lower chambers and connected to chamber(s) through electrolyte bridges, and reference or recording electrodes introduced into tubing attached to upper or lower chambers. The present invention also encompasses manufacture procedures and features for enhancing efficiency or accuracy of manufacture of devices and devices disclosed herein and devices made using such methods, including tapering of upper chamber wells, geometry of holes drilled into chips, ion transport measuring holes comprising one or more counterbores in chips, treatment of chips to enhance electrical sealing of particles such as cells, etc.

The present invention also includes methods of using an ion transport measuring device of the present invention having one or more flow-through lower chambers to measure one or more ion transport properties or activities of a cell or particle (such as, for example, a membrane vesicle). The methods include using a device that comprises at least upper chamber reversibly or irreversibly attached to a chip that comprises at least one ion transport measuring means in the form of a hole through the biochip, wherein the chip has been treated to have enhanced sealing properties, and at least one flow-through lower chamber. In the assembled devices used in the methods of the present invention, the holes of the biochip access the at least one flow-through lower chamber. In these methods, an upper chamber piece and chip are reversibly or irreversibly attached to a lower chamber piece that forms all or a portion of a flow-through lower chamber. An upper chamber piece and chip are optionally additionally reversibly attached to a lower chamber base piece that can form at least the lower surface of one or more lower chambers. Preferably, an upper chamber piece and chip are attached to at least one lower chamber piece that forms the walls of one or more lower chambers and at least one lower chamber base piece that forms the lower surfaces of one or more lower chambers and comprises conduits for the inflow and outflow of solutions.

The device is assembled such that the one or more upper chambers are in register with the one or more ion transport measuring holes of the chip, and one or more lower chambers access the one or more upper chambers via the one or more holes of the chip. In preferred embodiments, each of the one or more upper chambers is in register with one of the ion transport measuring holes of the chip, and each of the lower chambers is aligned with one upper chamber that it accesses via an ion transport measuring hole. Each of the lower chambers is connected to at least one inflow conduit and at least one outflow conduit.

During use of the device, the one or more upper chambers comprise, contact, or are in electrical contact with at least one electrode. During use of the device, the one or more lower chambers comprise, contact, or are in electrical contact with at least one electrode. In one alternative, the one or more upper chambers contact, comprise, or are in electrical contact with a common reference electrode, and the one or more lower chambers contact, comprise, or are in electrical contact with a individual reference electrodes. In another alternative, the one or more upper chambers contact, comprise, or are in electrical contact with individual reference electrodes, and the one or more lower chambers contact, comprise, or are in electrical contact with a common reference electrode.

The method includes: filling at least one flow-through lower chamber of the device with a measuring solution; adding at least one cell or at least one particle to one or more of the at least one upper chamber of the device, wherein the one or more upper chambers is connected to one of the at least one lower chambers that comprises measuring solution via a hole in the ion transport measuring chip; applying pressure to at least one flow-through lower chamber, at least one upper chamber, or to an upper chamber and a lower chamber that are connected via an ion transport measuring hole to create a high-resistance electrical seal between at least one cell or particle and at least one hole of the biochip; and measuring at least one ion transport property or activity of the at least one cell or at least one particle.

Preferably, one or more cells or one or more particles are in a suspension when added to the upper chamber. Various measuring solutions and, optionally, compounds In some preferred embodiments, the methods measure at least one ion transport activity or property of a cell in the whole cell configuration, but this is not a requirement of the present invention, as the devices can be used in a variety of applications on particles such as, for example, vesicles, as well as cells.

The application of pressure can be manual or automated. If pressure is applied manually (for example, by means of a syringe), preferably the user can make use of a pressure display system to monitor the applied pressure. Automated application of pressure can be through the use of a software program that is able to receive feedback from the device and direct and control the amount of pressure applied to one or more ion transport measuring units.

Various specific ion transport assay can be used for determining ion transport function or properties. These include methods known in the art such as but not limited to patch clamp recording, whole cell recording, perforated patch whole cell recording, vesicle recording, outside out or inside out recording, single channel recording, artificial membrane channel recording, voltage-gated ion transport recording, ligand-gated ion transport recording, recording of energy requiring ion transports (such as ATP), non energy requiring transporters, toxins such a scorpion toxins, viruses, stretch-gated ion transports, and the like. See, generally Neher and Sakman, Scientific American 266:44-51 (1992); Sakman and Neher, Ann. Rev. Physiol. 46:455-472 (1984); Cahalan and Neher, Methods in Enzymology 207:3-14 (1992); Levis and Rae, Methods in Enzymology 207:14-66 (1992); Armstrong and Gilly, Methods in Enzymology 207:100-122 (1992); Heinmann and Conti, Methods in Enzymology 207:131-148 (1992); Bean, Methods in Enzymology 207:181-193 (1992); Leim et al., Neurosurgery 36:382-392 (1995); Lester, Ann. Rev. Physiol 53:477-496 (1991); Hamill and McBride, Ann. Rev. Physiol 59:621-631 (1997); Bustamante and Varranda, Brazilian Journal 31:333-354 (1998); Martinez-Pardon and Ferrus, Current Topics in Developmental Biol. 36:303-312 (1998); Herness, Physiology and Behavior 69:17-27 (2000); U.S. Pat. No. 6,117,291; U.S. Pat. No. 6,107,066; U.S. Pat. No. 5,840,041 and U.S. Pat. No. 5,661,035; Boulton et al., Patch-Clamp Applications and Protocols, Neuromethods V. 26 (1995), Humana Press, New Jersey; Ashcroft, Ion Channels and Disease, Cannelopathies, Academic Press, San Diego (2000); Sakman and Neher, Single Channel Recording, second edition, Plenuim Press, New York (1995) and Soria and Cena, Ion Channel Pharmacology, Oxford University Press, New York (1998), each of which is incorporated by reference herein in their entirety.

During the assay, while the cell or particle maintains a high-resistance seal with the ion transport measuring hole, lower chamber solutions such as intracellular solutions can be exchanged using the inflow and outflow conduits. For example, a given patch-clamped cell can be assayed without drug, after addition of drug, and after washout of drug while maintaining a high-resistance seal. In another example, a cell or particle can be assayed for ion transport activity in the presence and absence of a particular ion by means of exchange of the lower chamber solution.

III. Method of Making an Upper Chamber Piece of a Device for Ion Transport Measurement

In ion transport measuring devices contemplated by the present invention, an upper chamber is designed to contain the cells or particles on which ion transport measurements are to be performed. In these embodiments, an upper chamber of an ion transport measuring device can comprise or engage at least a portion of an electrode used to monitor ion transport activity. In the alternative, an upper chamber, when filled with an ion transport measuring solution, can be brought into electrical contact with at least a portion of an electrode. For example, an electrode (such as, but not limited to, a metal wire) can be inserted into the well so that electrical current from the electrode would be transmitted through the conductive measuring solution. Alternatively, a tube that comprises a measuring solution (or otherwise conductive solution) that contains or contacts an electrode or a portion thereof can be put in contact with the upper chamber solution. In the latter case, the electrode (or a portion thereof) need not be within the upper chamber at all, as long as it is electrically connected to the inner part of the upper chamber conductive solution (electrolyte bridge).

Typically, an upper chamber electrode will be a reference electrode, although this need not be the case. In cases in which upper chamber electrodes are reference electrodes, electrode extensions or electrolyte bridges that contact individual upper chambers can be connected with one another either outside or inside the upper chamber piece.

In many of the devices of the present invention, an upper chamber piece comprises at least one upper chamber in the form of a well. Preferably, an upper chamber piece comprises multiple upper chambers or wells that allow several ion transport assays to be performed simultaneously. The upper chamber piece can optionally comprise one or more electrodes. The present invention provides methods of making upper chamber pieces that increase the efficiency and reduce the cost of making devices that measure ion transport activity of cells and particles.

Two-Piece Molding Followed by Electrode Insertion

In one aspect of the present invention, an upper chamber piece that comprises one or more wells is made in two pieces, an upper well portion piece and a well hole portion piece, and the well hole portion piece has a groove into which a wire electrode can fit. An upper well portion piece comprises the upper portion of one or more wells. The upper well portions are open at both ends. The well hole piece comprises one or more well holes that will form the bottom portion of the one or more wells. A well hole is, in effect, the lower portion of a well and can have different dimensions (height, diameter, and taper angle) than the upper well portion. The well holes are also open at their upper and lower ends. The well holes have an upper diameter that is equal or smaller than the diameter of the lower opening of the upper well portion. When the upper well portion piece is attached on top of the well hole piece, the upper well portions are aligned over the well holes to form upper chambers (wells) that have well holes at their lower end.

After manufacturing the upper well portion piece and the well hole piece, a wire electrode is inserted into the groove of the well hole piece, and then the upper well portion piece is attached, via, for example ultrasonic welding, to the well hole piece to form an upper chamber piece comprising one or more wells, each of which is in contact with a portion of a wire electrode.

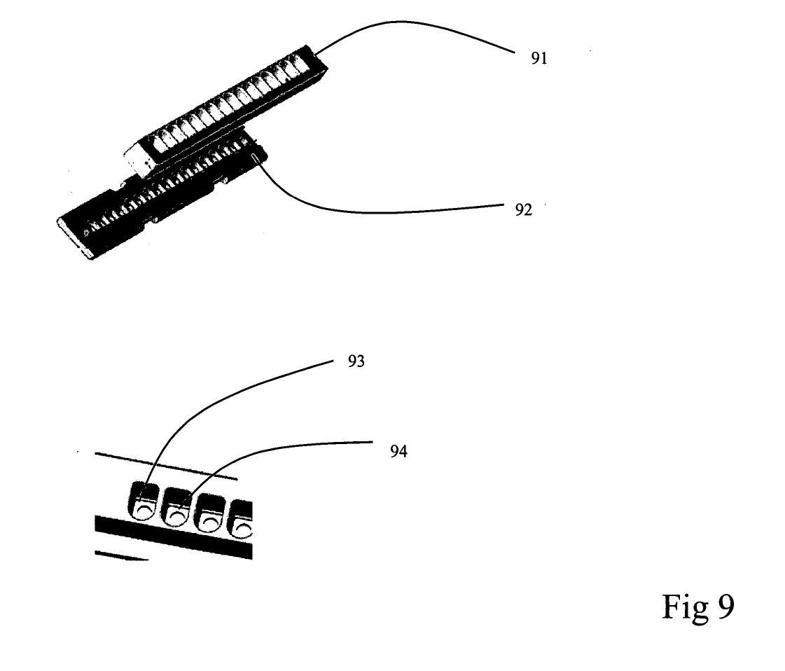

An example of this manufacture (an upper well piece made by assembling an upper well portion piece having upper portions of wells with an upper well hole piece having well holes) is depicted in FIG. 9. In FIG. 9A, the upper well portion piece (91) is shown suspended above the well-hole piece (92). The groove (94) into which a wire electrode can fit is seen along the backs of the wells (93) in the assembled upper well piece shown in FIG. 9B.

The method includes: molding a well hole portion piece of an upper chamber piece of an ion transport measuring device, wherein said well hole portion piece comprises: at least one well hole, and a groove that extends longitudinally from one end of the well hole portion piece toward the opposite end of the well hole portion piece, such that the groove contacts the one or more well holes; molding an upper well portion piece of an upper chamber piece that comprises at least one upper well; inserting a wire electrode into the groove of the well hole portion piece; and attaching the upper well portion piece to the well hole portion piece to form an upper chamber piece that comprises one or more wells, such that the wire electrode is exposed to the interior of said one or more wells.

In this embodiment, the upper piece is made from one or more plastics and comprises wells that are open at their upper and lower ends, and each well contacts or contains a portion of a common electrode that can be used as a reference electrode in ion transport measuring assays. This method of manufacture is particularly suited to embodiments where the upper piece comprises multiple wells (at least two) that will contact a common electrode, and wells are arranged linearly in a row. However, this is not a requirement of the present invention, and the principle of two-piece molding and wire insertion can be adapted to the manufacture of device components in which multiple wells or chambers that will share a common electrode are arranged in different geometries. In such embodiments, the path of the groove can be designed such that it contacts all of the wells or chambers that are intended to be in contact with the electrode. This includes embodiments where there are multiple rows of wells or chambers, arrangement of wells or chambers in concentric circles or spirals as well as other arrangements of wells or chambers.

It is also possible to adapt the methods of the present invention to designs in which one or more wells are to be contacted by one electrode and one or more other wells are to be contacted by a different electrode. It is also possible that one well be contacted with more that one electrode. In such cases, the well hole portion piece will comprise more than one continuous groove such that more than one wire electrode can be inserted into the lower well portion piece.

Injection molding or compression molding techniques as they are known in the art can be used to make the well hole portion piece and the upper well portion piece. In the methods of the present invention, the upper well portion piece comprises an upper portion of at least one well or chamber and the well hole portion piece comprises a lower portion of at least one well or chamber, such that when the upper well portion piece is attached to the well hole portion piece, the two pieces together form at least one upper well or upper chamber. The well hole portion piece comprises at least one groove whose diameter corresponds to that of a wire electrode, and the groove contacts the well holes. Preferably, the well hole portion piece comprises a well hole whose upper diameter is equal to or smaller than the lower diameter of the upper portion of the well that is part of the upper well portion piece. Thus, in preferred embodiments, the well hole portion piece will have a top surface around the upper diameter of the well hole (see FIG. 9), that, when looking down into a well after the entire top chamber piece is assembled, appears as a ledge around the top of the well hole. The groove can be in this top surface or ledge. In this way the wire electrode can be easily inserted into the groove, and its placement on this “ledge” ensures that it will be exposed to the interior of the well after attachment of the upper well portion piece.

The wire is easily inserted into the groove of the lower well portion piece, as the groove is totally accessible prior to attachment the upper and lower portion pieces.

After insertion of the wire electrode, the upper well portion piece and well hole portion piece are fused together to form a complete upper chamber piece. Any glues appropriate to the materials and applications of the devices can be used for this purpose. UV glues and other fast-curing glues are preferred for mass production of the upper chamber pieces, although slow-cure glues can also be used for mass production if a high capacity production process is used. Ultrasonic welding, pressuring, heating, or other bonding methods can also be used.

Upper Chamber Pieces and Devices

The present invention includes upper chamber pieces that are made using the methods of the present invention, and devices that comprise such pieces. Such pieces and devices can comprise wells or chambers that are open or closed at one or both ends, can comprise other components, such as, but not limited to, membranes, microstructures, ports (optionally with attached conduits), fluidic channels, particles positioning means, specific binding members, polymers, etc., and are not limited to use as ion transport measuring devices. In fact, the same design and manufacturing principles can be used to fabricate pieces that comprise wells or chambers that need not function as “upper” pieces of devices or apparatuses. Two-piece molding, wire insertion, and attachment of two pieces can be used to make devices or components of devices that comprise wells or chambers regardless of whether the components, chambers, or wells, can be considered “upper”.

Plastics that can be used in the manufacture of upper and lower pieces include, but are not limited to polyallomer, polypropylene, polystyrene, polycarbonate, cyclo olefin polymers (e.g., Zeonor®), polyimide, paralene, PDMS, polyphenylene ether/PPO or modified polyphenylene oxide (e.g., Noryl®), etc. A very large number and variety of moldable plastics and their properties are known.

Electrodes can comprise conductive materials such as metals that can be shaped into wires. Various metals, including aluminum, chromium, copper, gold, nickel, palladium, platinum, silver, steel, and tin can be used as electrode materials. For electrodes used in ion channel measurement, wires made of silver or other metal halides are preferred, such as Ag/AgCl wires.

The design and dimensions of the upper and lower well pieces, as well as the dimension of the upper wells and lower wells, can vary according to the preferences of the user and are not limiting to the present invention.

Preferred Embodiments: Upper Chamber Pieces and Devices

In preferred embodiments of the present invention, the upper chamber piece comprises one or more upper wells that can function as the upper chambers of ion transport measuring units of ion transport measuring devices. Preferably, an upper chamber piece of the present invention comprises more than one upper well, and more preferably more than two upper wells. Even more preferably, an upper chamber piece comprises six or more upper wells, each of which can be a part of an ion transport measuring unit of an ion transport measuring device, where all of the six or more upper wells of the manufactured upper chamber piece contact a portion of a common wire electrode that extends along the upper chamber piece.

The wells of an upper chamber piece that can be part of an ion transport measuring device preferably can hold a volume of between about 5 microliters and about 5 milliliters, more preferably between about 10 microliters and about 2 milliliters, and more preferably yet between about 25 microliters and about 1 milliliter. The depth, or height of a well can vary from about 0.01 to about 25 millimeters or more, and more preferably will be from about 2 milliliters to about 10 milliliters or more in depth. In preferred embodiments of the present invention in which an upper well portion and a lower well portion together make up the well, the upper well portion is preferably from about 1 to about 25 milliliters in depth, and the lower well is preferably from about 100 microns to about 10 milliliters in depth.

A low cell or particle density is often preferred for attaining a high success rate when using the ion channel measuring device described herein. In order to reduce the cell or particle density required for optimal cell or particle landing to the recording apertures, it is desirable to have an accurate means for delivering the cells or particles to the recording aperture. For a more accurate delivery of cells or particles to the recording aperture, the upper chamber well can have one or more tapered walls, The walls can be contoured such that the cells or particles, when delivered to the upper chamber well wall (such as by robotic dispenser), are directed to the recording aperture.

In these preferred embodiments, the shape of the well can vary, and can be irregular or regular, and in many cases will be generally circular or oval at its circumference. In preferred embodiments, the diameter of a well at its upper end will generally be from about 2 millimeter to about 10 millimeters. In some preferred embodiments of the present invention such as those depicted in FIG. 1 and FIG. 9, the upper circumferences of the wells of the upper chamber piece are horseshoe-shaped, and at least a portion of the sides of the wells are tapered. FIG. 1D, for example, shows that the wall of the well (1) corresponding to the rounded end of the horseshoe shape tapers toward the bottom of the well. In other preferred embodiments, the walls along entire well can taper toward the bottom of the upper portion of the well. In some preferred embodiments of the present invention the angle of the taper of a portion of the walls of the well or the entire well walls (the difference from vertical) is from about one degree to about 80 degrees. More preferably, the angle of the taper of the well walls is between about 5 degrees and 60 degrees from vertical. The taper can extend down the full height of the well, or the well can be tapered for only a portion of its height. The upper well portion can optionally be tapered, or the well hole can optionally be tapered, or both the upper well portion and the lower well portion can be tapered. Where both are tapered, the tapering need not be to the same degree or extend around the well to the same extent.

Molding of Single Upper Chamber Piece Around Electrode

In another aspect of the present invention, an upper chamber piece with at least one wire electrode can be manufactured as a single piece by molding an upper piece around a wire electrode. In this case, the mold has a means for positioning the wire electrode such that the upper chamber piece that includes the wells can be molded around it. The method includes: positioning an electrode in a mold; and injection molding an upper chamber piece using the mold such that the electrode contacts one or more wells of the upper chamber piece. The electrode can be positioned in any of a number of ways, for example it can extend through the mold and be held by apertures that it is threaded through on either end of the mold.

The injection molded upper chamber piece can comprise one or more wells or upper chambers, preferably two or more, more preferably six or more wells. The wells can be of any dimension of size, and can comprise a well hole within the well as described in the previous section.

Molding of Single Upper Piece without Electrode

In yet another aspect of the present invention, an upper chamber piece can be manufactured without an electrode. In this case, an upper chamber piece with a desirable number of wells is injection molded using a suitable plastic, such as, but not limited to, polyallomer, polypropylene, polystyrene, polycarbonate, polyimide, paralene, PDMS, cyclo olefin polymers (for example, Zeonor®, or polyphenylene ether/PPO or modified polyphenylene oxides (for example, Noryl®).

When the upper chamber piece is integrated into a device for ion transport measurement, electrodes (for example, metal wires) can be inserted into the wells. Such electrodes are preferably reference electrodes and are preferably connected outside the chambers, but inserted electrodes can also be recording electrodes connected separately to a power source/signal amplifier.

In a preferred embodiment of the present invention, an electrode connection can be provided by a conduit that can be introduced into the upper chambers during use of the device. The conduit can comprise an electrode, or, when the conduit is filled with a conductive solution, can be in electrical contact with an electrode. When both the upper chamber and the conduit contain a conductive solution (such as a measuring solution), the upper chamber is in electrical contact with the electrode through the “electrolyte bridge” of solution provided by the conduit.

Insert Molding of Glass Chip

In yet another embodiment, a pre-diced glass chip is insert-molded together with an upper chamber piece to make a one-piece cartridge. In this process, a glass chip is inserted into a mold, and the upper chamber piece is molded around the glass chip such that it forms the bottoms of upper chambers of the upper chamber piece. Laser drilling of the recording apertures is done after the molding process, and then the cartridge is chemically treated to enhance its electrical sealing properties. In this embodiment, materials that can be treated with acid and base (such as, for example, polyphenylene ether/PPO or modified polyphenylene oxide (e.g., NORYL®) and cylco olefin polymers (e.g.,ZEONOR®) are used for the construction of the cartridge other than the biochip.

Additional Features

In some preferred embodiments of the present invention, the upper chamber pieces of the present invention or components of the upper chamber pieces of the present invention can have additional features that can aid in the manufacture of upper chamber pieces or of ion transport measuring devices. One such feature is an alignment bump (also called a registration edge) (2) as seen on the chamber piece depicted in FIG. 1B. One or more alignment bumps on the lower surface of an upper chamber piece can be used during attachment of a chip that comprises ion transport measuring means to the upper chamber piece. Attachment of the chip and the upper chamber piece must occur such that every ion transport measuring hole in the chip is aligned with a well hole. The alignment bump or registration edge allows a person or machine assembling the device to detect the location where the edge of the chip must be positioned.

Another useful feature for the manufacture of ion transport measuring devices that can occur on upper chamber piece of the present invention is a glue spillage groove. This allows for overflow of glue that is used for the attachment of a chip, such as a chip that comprises ion transport measuring means. The glue spillage groove (4) is also shown as a notch in the bottom surface of the part shown in FIG. 1D.

Yet another optional feature useful in the manufacturing process of an upper chamber piece is the presence of sinkholes. Depicted in FIG. 1C, these sinkholes (3) allow for appropriate expansion and contraction of the piece during molding.

IV. Methods of Making a Chip Comprising Holes for Ion Transport Measurement

Fabrication of Ion Transport Measuring Holes in a Chip

For optimal quality ion transport recording, ion transport measurement chips comprising holes for ion transport measurement ideally should have a low hole resistance (Re) across the chip. For chips having multiple holes, it is also desirable to have a high degree of uniformity of Re from recording site to recording site. It is also desirable to have ion transport measuring chips that can form seals of the ion transport measuring holes of the chip with a cell membrane such that the seal resistance (R) is high and the access resistance (Ra) is low.

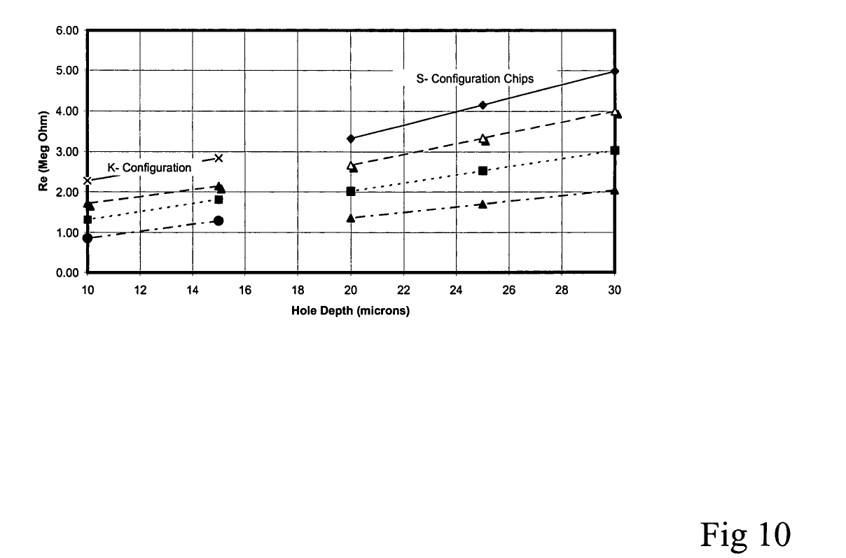

Chip geometry determines hole resistance (Re) which in turn determines the lowest achievable Ra. FIG. 10 shows that chips of the present invention having shallower holes and reduced entrance hole diameters (known as “K configuration chips” or “K chips”), have reduced Re relative to standard chips (“S configuration chips” or “S chips”). FIG. 10 demonstrates that for S chips, the Re of seals (y-axis) decreases with increasing width of the exit hole (opening at the lower side of the chip), and increases with increasing hole depth (x-axis). For K chips, the same relationship holds, however the Re of seals of K chips is lower than those of comparable S chips having holes with the same exit hole diameters (comparing the K configuration chips on the left side of the graph with the S configuration chips on the right side of the graph.) A wider tapering (greater angle from vertical) of the hole also decreases Re.

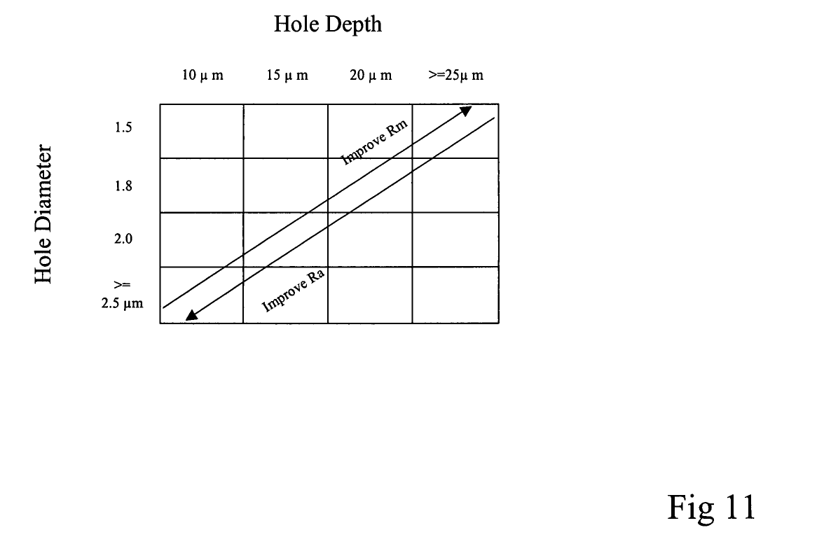

FIG. 11 also shows that the Ra of a seal on a chip decreases with decreasing depth of the hole in the chip and widening of the exit hole. Improved Ra, however, comes at the expense of reduced seal resistance (here, Rm).

The present invention includes methods of making chips that can form seals with cells and cell membranes such that the seals have low access resistance and high seal resistance. The methods of the present invention seek to reduce hole resistance (Re) of ion transport measuring holes of chips by reducing hole depth. This is achieved by laser drilling holes in thin substrates, such as glass, quartz, silicon, silicon dioxide, or polymer substrates.

A chip with shortened holes for ion transport measurement can be made by laser drilling one or more counterbores into a glass chip, and then laser drilling a through-hole through the one or more counterbores. While a wide counterbore is preferred for lower Re, increased width of the counterbore can weaken the chip. It is also difficult to control the drilling of the counterbore as the bottom of the counterbore gets thinner and thinner. In addition, with increased (deeper) drilling, the peripheral areas of the counterbores tend to be deeper than the more central portions of the counterbore due to optical effects (this is sometimes called the wave guide effect). To avoid these problems, a second counterbore is laser drilled into the bottom of a first counterbore. This makes drilling to a greater depth easier control, and has the effect of reducing the thickness of the chip in the vicinity of the through-hole. Thus, preferred methods for synthesis of biochips for ion transport measurement include laser drilling at least one counterbore through a substrate, and then drilling a through-hole through the one or more counterbores. Preferably two counterbores are laser drilled into a substrate, such that a second counterbore is drilled through a first counterbore, that is, the counterbores are nested to form (along with a through-hole) a single hole structure. In some embodiments of the present invention, three, four, or more nested counterbores can be drilled into a substrate prior to drilling a through-hole through the counterbores.

Control of the depth of laser drilling can be done by using a separate laser device that can measure the thickness of the glass. In preferred aspects of this embodiment of the present invention, a measuring laser is used to measure the thickness of the substrate before or as it is being drilled, and the laser used for drilling can be regulated by the thickness of the remaining substrate at the bottom surface of the counterbore. Laser-based measuring devices have been used for the determination of glass thickness to an accuracy of 0.1 micron. Such a laser measurement device is available from the Keyence Company. A laser based measurement is made to determine the exact thickness of the substrate. This measurement determines the number of pulses to be used by the drilling laser to drill a counterbore and thereby achieve uniformity of hole depth. To improve the consistency of through-hole depth and hole resistance, the invention contemplates the integration of a laser unit with an excimer laser drilling device, together with automated control software.

Thus, the present invention comprises methods of making chips comprising holes for ion transport measurement that can form seals having a high seal resistance and low access resistance with cells and particles. The method includes: providing a substrate; laser drilling at least one counterbore in the substrate, and laser drilling at least one hole through the counterbore in the substrate. Preferably, laser drilling is done with sequential or simultaneous measurement of the glass thickness at the site of the pore.

In practice, a substrate made of glass, quartz, silicon, silicon dioxide, polymers, or other substrates that preferably ranges in thickness from 5 to 1000 microns, and more preferably from 10 to 200 microns, is provided. A first counterbore is laser drilled, where the entrance of the counterbore has a diameter from about 20 to about 200 microns, preferably from about 40 to about 120 microns. The first counterbore can be drilled to a depth of the thickness of the substrate minus the through-hole depth, with the depth depending on the thickness of the substrate and the number of counterbores that each ion transport measuring hole will have. Subsequent counterbores will have a smaller diameter than the first counterbore, and can be of lesser depth than the first counterbore. In general, after drilling of all of the counterbores that will be part of an ion transport measuring hole, the remaining thickness of the substrate that is to be drilled out to form the through-hole (that is, the depth of the through-hole) will range from about 0.5 to about 200 microns, and preferably will range from about 2 to about 50 microns, more preferably from about 5 to about 30 microns. The diameter of the through-hole can be from about 0.2 to about 8 microns, and preferably will be from about 0.5 to about 5 microns, and even more preferably, from about 0.5 to about 3 microns.

Counterbores can be tapered. Preferably, a counterbore is tapered at an angle ranging from about 1 degree to about 80 degrees from vertical, and more preferably from about 3 degrees to about 45 degrees from vertical. Ion transport measuring holes comprising multiple counterbores can have different taper angles for different counterbores.

Through-holes can also be tapered. The angle of taper for a through-hole can range from about 0 degree to about 75 degrees from vertical, and more preferably, where a through-hole is tapered, is from about 0 degree to about 45 degrees from vertical. In general an exit hole of a through-hole will have a narrower diameter than an entrance hole, although this is not a requirement of the present invention.

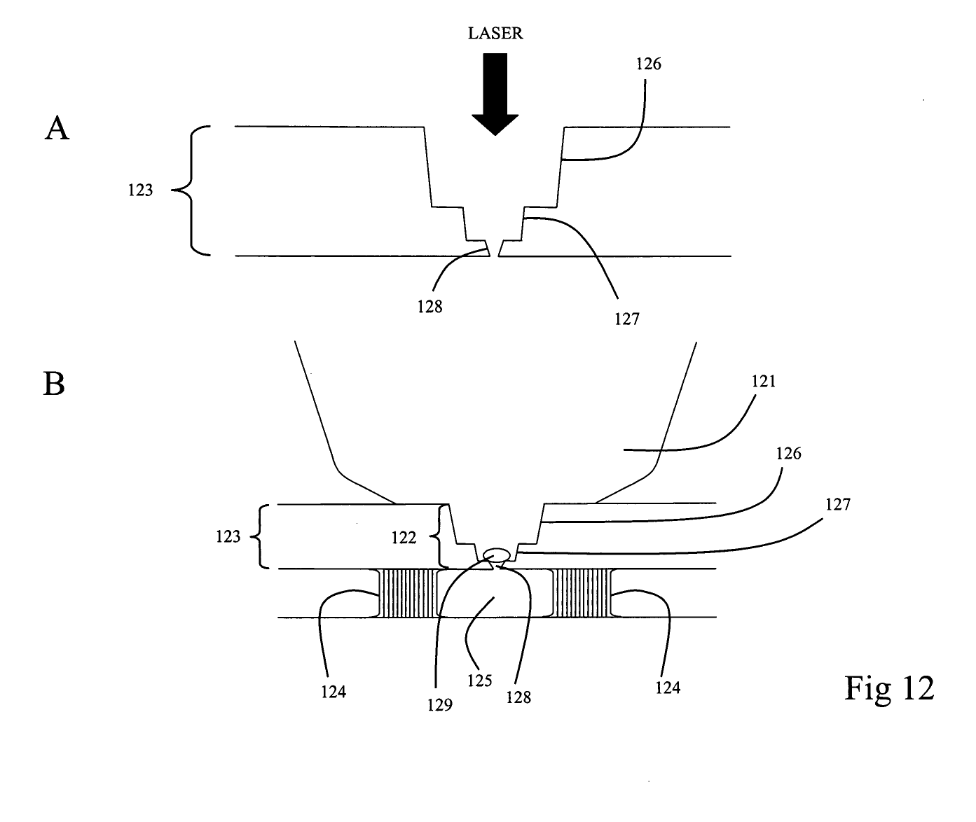

The present invention includes chips made using the methods of the present invention having at least one counterbore and at least one through-hole drilled through the counterbore. FIG. 12A depicts a chip of the present invention (123) having a laser drilled ion transport measuring means that comprises a first counterbore (126), a second counterbore (127), and a through-hole (128).

Preferably, the chips of the present invention that comprise through holes laser drilled through counterbores have electrical sealing properties such that when appropriate pressure is applied to achieve a seal, a seal between the chip and a cell or particle has a seal resistance (R) that is greater than the resistance across the hole (Re). Preferably, the chips produced by the methods of the present invention have ion transport measuring holes that are able to seal to cells or cell membranes such that electrical access between said chip an the inside of said cell or particle, or between said chip and the outside of said cell or particle in the region of said hole has an access resistance (Ra) that is less than the seal resistance (R). Preferably, the seal between the ion transport measuring hole of a chip made by the methods of the present invention and a cell or cell membrane has a seal resistance that is at least 200 MOhm, more preferably at least 500 MOhm, and more preferably yet one gigaOhm or greater.

In preferred embodiments of chips of the present invention having at least one ion transport measuring means comprising at least one laser drilled counterbore and a through-hole laser drilled through the one or more counterbores, the chip has been treated to enhance the electrical sealing properties of the chip. Preferably, the chip has been treated to make the surface of the chip at or near the ion transport measuring hole or holes more electronegative. For example, chips of the present invention can be chemically treated, such as by methods described herein, to become more electronegative.

Preferably, a chip made by the methods of the present invention can produce a seal with a cell or particle that has an access resistance that is less than 80 MOhm, more preferably less than about 30 MOhm, and more preferably yet, less than about 10 MOhm. Preferably, a chip of the present invention comprising at least one ion transport measuring means in the form of a through-hole that has been laser-drilled through at least one counterbore can form a seal with a cell such that the resistance of the seal is at least ten times the access resistance. More preferably, a chip of the present invention can form a seal with a cell such that the seal resistance is at least twenty times the access resistance.

A chip produced by methods of the present invention can be used in any ion transport measuring device, including but not limited to those described herein.

Inverted Chip

The present invention also includes methods of using chips comprising ion transport measuring holes that are in inverted orientation for ion transport measurement, that is, using chips in which the holes (or at least a portion of the holes, such as a portion of the holes made by at least one counterbore) have a negative taper.

The method comprises: assembling a device for ion transport measurement that comprises: at least one upper chamber, wherein the one or more upper chambers comprise or are in electrical contact with at least one electrode; at least one chip that comprises an ion transport measuring hole, wherein the one or more chips are assembled in the device in inverted orientation; and at least one lower chamber, wherein the one or more lower chambers comprise or are in electrical contact with at least one electrode; connecting the electrodes with a power supply/signal amplifier; introducing at least one particle or at least one cell into at least one upper chamber, and measuring ion transport activity of at least one cell or at least one particle.

By “inverted orientation” is meant that, for a chip in which ion transport measuring holes are made by drilling, the chip is positioned such that the side of the chip having the laser entrance hole opening is exposed to a chamber that will contain cells or particles, instead of the side having the laser exit hole. This is contrary to what has previously been done in the art—the “upside-up” orientation in which the cells or particles seal against the side of the chip that has the laser exit hole. Thus, sealing of a cell or particles against the ion transport measuring hole occurs on the side of the chip opposite to the side that has smaller hole size (the “back side” of the chip).

The inverted chip orientation has several advantages. One advantage is that the chip does not require a laser polishing step, since the laser drilling performs this function as a “side-effect”. A second advantage is that sealing occurs with high efficiency due to the geometry of the particle-chip interaction. Yet another advantage is that a stable low Ra can be produced using larger holes (for example, from about 2 to about 5 microns in diameter), due to the position at which break-in occurs during whole cell recording.

When one or counterbores are used to reduced the through-hole depth, the through-hole can be drilled from either the same direction as the counterbores, or from the opposite direction to the counterbores. In the former case, the chips is produced just like the “normal” chips are produced, they are simply assembled up side down. FIG. 12B illustrates the use of a chip with laser drilled counterbores (126, 127) and through-hole (128) used in inverted orientation. The single unit of the ion transport measuring device shown has an upper well (121) attached to a chip (123) comprising an ion transport measuring means in the form of a hole (122) that connects the upper chamber (121) with a lower chamber (125). In this case, a gasket (124) forms the walls of the lower chamber. A cell (129) is shown sealed to the through-hole (128) of the chip which is being used in inverted orientation.

The present invention includes devices and apparatuses having chips comprising ion transport measuring holes that are in inverted orientation, as well as methods of using chips comprising ion transport measuring holes that are in inverted orientation for ion transport measurement.

Methods of Treating Chips Comprising Ion Transport Measuring Means to Enhance the Electrical Seal of a Particle

The present invention also includes methods of modifying an ion transport measuring means to enhance the electrical seal of a particle or membrane with the ion transport measuring means. Ion transport measuring means includes, as non-limiting examples, holes, apertures, capillaries, and needles. “Modifying an ion transport measuring means” means modifying at least a portion of the surface of a chip, substrate, coating, channel, or other structure that comprises or surrounds the ion transport measuring means. The modification may refer to the surface surrounding all or a portion of the ion transport measuring means. For example, a biochip of the present invention that comprises an ion transport measuring means can be modified on one or both surfaces (e.g. upper and lower surfaces) that surround an ion transport measuring hole, and the modification may or may not extend through all or a part of the surface surrounding the portion of the hole that extends through the chip. Similarly, for capillaries, pipettes, or for channels or tube structures that comprises ion transport measuring means (such as apertures), the inner surface, outer surface, or both, of the channel, tube, capillary, or pipette can be modified, and all or a portion of the surface that surrounds the inner aperture and extends through the substrate (and optionally, coating) material can also be modified. Methods of modifying an ion transport measuring means to enhance the electrical seal of a particle or membrane with the ion transport measuring means are also disclosed in U.S. patent application Ser. No. 10/760,866 filed Jan. 20, 2004, and U.S. patent application Ser. No. 10/642,014, filed Aug. 16, 2003, both of which are herein incorporated by reference in their entireties.

As used herein, “enhance the electrical seal”, “enhance the electric seal”, “enhance the electric sealing” or “enhance the electrical sealing properties (of a chip or an ion transport measuring means)” means increase the resistance of an electrical seal that can be achieved using an ion transport measuring means, increase the efficiency of obtaining a high resistance electrical seal (for example, reducing the time necessary to obtain one or more high resistance electrical seals), or increasing the probability of obtaining a high resistance electrical seal (for example, the number of high resistance seals obtained within a given time period).

The method comprises: providing an ion transport measuring means and treating the ion transport measuring means to enhance the electrical sealing properties of the ion transport measuring means. Preferably, treating an ion transport measuring means to enhance the electrical sealing properties results in a change in surface properties of the ion transport measuring means. The change in surface properties can be a change in surface texture, a change in surface cleanness, a change in surface composition such as ion composition, a change in surface adhesion properties, or a change in surface electric charge on the surface of the ion transport measuring means. In some preferred aspects of the present invention, a substrate or structure that comprises an ion transport measuring means is subjected to chemical treatment (for example, treated in acid, and/or base for specified lengths of time under specified conditions). For example, treatment of a glass chip comprising a hole through the chip as an ion transport measuring means with acid and/or base solutions may result in a cleaner and smoother surface in terms of surface texture for the hole. In addition, treating a surface of a biochip or fluidic channel that comprises an ion transport measuring means (such as a hole or aperture) or treating the surface of a pipette or capillary with acid and/or base may alter the surface composition, and/or modify surface hydrophobicity and/or change surface charge density and/or surface charge polarity.

Preferably, the altered surface properties improve or facilitate a high resistance electric seal or high resistance electric sealing between the surface-modified ion transport measuring means and a membranes or particle. In preferred embodiments of the present invention in which the ion transport measuring means are holes through one or more biochips, one or more biochips having ion transport measuring means with enhanced sealing properties (or, simply, a “biochip having enhanced sealing properties”) preferably has a rate of at least 50% high resistance sealing, in which a seal of 1 Giga Ohm or greater occurs at 50% of the ion transport measuring means takes place in under 2 minutes after a cell lands on an ion transport measuring hole, and preferably within 10 seconds, and more preferably, in 2 seconds or less. Preferably, for biochips with enhanced sealing properties, a 1 Giga Ohm resistance seal is maintained for at least 3 seconds.

In practice, in preferred aspects of the present invention the method comprises providing an ion transport measuring means and treating the ion transport measuring means with one or more of the following: heat, a laser, microwave radiation, high energy radiation, salts, reactive compounds, oxidizing agents (for example, peroxide, oxygen plasma), acids, or bases. Preferably, an ion transport measuring means or a structure (as nonlimiting examples, a structure can be a substrate, chip, tube, or channel, any of which can optionally comprise a coating) that comprises at least one ion transport measuring means is treated with one or more agents to alter the surface properties of the ion transport measuring means to make at least a portion of the surface of the ion transport measuring means smoother, cleaner, or more electronegative.

An ion transport measuring means can be any ion transport measuring means, including a pipette, hole, aperture, or capillary. An aperture can be any aperture, including an aperture in a channel, such as within the diameter of a channel (for example, a narrowing of a channel), in the wall of a channel, or where a channel forms a junction with another channel. (As used herein, “channel” also includes subchannels.) In some preferred aspects of the present invention, the ion transport measuring means is on a biochip, on a planar structure, but the ion transport measuring means can also be on a non-planar structure.

The ion transport measuring means or surface surrounding the ion transport measuring means modified to enhance electrical sealing can comprise any suitable material. Preferred materials include silica, glass, quartz, silicon, plastic materials, polydimethylsiloxane (PDMS), or oxygen plasma treated PDMS. In some preferred aspects of the present invention, the ion transport measuring means comprises SiOM surface groups, where M can be hydrogen or a metal, such as, for example, Na, K, Mg, Ca, etc. In such cases, the surface density of said SiOM surface groups (or oxidized SiOM groups (SiO⁻)) is preferably more than about 1%, more preferably more than about 10%, and yet more preferably more than about 30%. The SiOM group can be on a surface, for example, that comprises glass, for example quartz glass or borosilicate glass, thermally oxidized SiO₂ on silicon, deposited SiO₂, deposited glass, polydimethylsiloxane (PDMS), or oxygen plasma treated PDMS.

In preferred embodiments, the method comprises treating said ion transport measuring means with acid, base, salt solutions, oxygen plasma, or peroxide, by treating with radiation, by heating (for example, baking or fire polishing) by laser polishing said ion transport measuring means, or by performing any combinations thereof.

An acid used for treating an ion transport measuring means can be any acid, as nonlimiting examples, HCl, H₂SO₄, NaHSO₄, HSO₄, HNO₃, HF, H₃PO₄, HBr, HCOOH, or CH₃COOH can be. The acid can be of a concentration about 0.1 M or greater, and preferably is about 0.5 M or higher in concentration, and more preferably greater than about 1 M in concentration. Optimal concentrations for treating an ion transport measuring means to enhance its electrical sealing properties can be determined empirically. The ion transport measuring means can be placed in a solution of acid for any length of time, preferably for more than one minute, and more preferably for more than about five minutes. Acid treatment can be done under any non-frozen and non-boiling temperature, preferably at greater or equal than room temperature.

An ion transport measuring means can be treated with a base, such as a basic solution, that can comprise, as nonlimiting examples, NaOH, KOH, Ba(OH)₂, LiOH, CsOH,or Ca(OH)₂. The basic solution can be of a concentration of about 0.01 M or greater, and preferably is greater than about 0.05 M, and more preferably greater than about 0.1 M in concentration. Optimal concentrations for treating an ion transport measuring means to enhance its electrical sealing properties can be determined empirically (see examples). The ion transport measuring means can be placed in a solution of base for any length of time, preferably for more than one minute, and more preferably for more than about five minutes. Base treatment can be done under any non-frozen and non-boiling temperature, preferably at greater or equal than room temperature.

An ion transport measuring means can be treated with a salt, such as a metal salt solution, that can comprise, as nonlimiting examples, NaCl, KCl, BaCl₂, LiCl, CsCl, Na₂SO₄, NaNO₃, or CaCl, etc. The salt solution can be of a concentration of about 0.1 M or greater, and preferably is greater than about 0.5 M, and more preferably greater than about 1 M in concentration. Optimal concentrations for treating an ion transport measuring means to enhance its electrical sealing properties can be determined empirically (see examples). The ion transport measuring means can be placed in a solution of metal salt for any length of time, preferably for more than one minute, and more preferably for more than about five minutes. Salt solution treatment can be done under any non-frozen and non-boiling temperature, preferably at greater or equal than room temperature.

Where treatments such as baking, fire polishing, or laser polishing are employed, they can be used to enhance the smoothness of a glass or silica surface. Where laser polishing of a chip or substrate is used to make the surface surrounding an ion transport measuring means more smooth, it can be performed on the front side of the chip, that is, the side of the chip or substrate that will be contacted by a sample comprising particles during the use of the ion transport measuring chip or device.

Appropriate temperatures and times for baking, and conditions for fire and laser polishing to achieve the desired smoothness for improved sealing properties of ion transport measuring means can be determined empirically.

In some aspects of the present invention, it can be preferred to rinse the ion transport measuring means, such as in water (for example, deionized water) or a buffered solution after acid or base treatment, or treatment with an oxidizing agent, and, preferably but optionally, before using the ion transport measuring means to perform electrophysiological measurements on membranes, cells, or portions of cells. Where more than one type of treatment is performed on an ion transport measuring means, rinses can also be performed between treatments, for example, between treatment with an oxidizing agent and an acid, or between treatment with an acid and a base. An ion transport measuring means can be rinsed in water or an aqueous solution that has a pH of between about 3.5 and about 10.5, and more preferably between about 5 and about 9. Nonlimiting examples of suitable aqueous solutions for rinsing ion transport measuring means can include salt solutions (where salt solutions can range in concentration from the micromolar range to 5M or more), biological buffer solutions, cell media, or dilutions or combinations thereof. Rinsing can be performed for any length of time, for example from minutes to hours.

Some preferred methods of treating an ion transport measuring means to enhance its electrical sealing properties include one or more treatments that make the surface more electronegative, such as treatment with a base, treatment with electron radiation, or treatment with plasma. Not intending to be limiting to any mechanism, base treatment can make a glass surface more electronegative. This phenomenon can be tested by measuring the degree of electro-osmosis of dyes in glass capillaries that have or have not been treated with base. In such tests, increasing the electronegativity of glass ion transport measuring means correlates with enhanced electrical sealing by the base-treated ion transport measuring means. Base treatment can optionally be combined with one or more other treatments, such as, for example, treatment with heat (such as by baking or fire polishing) or laser treatment, or treatment with acid, or both. Optionally, one or more rinses in water, a buffer, or a salt solution can be performed before or after any of the treatments.

For example, after manufacture of a glass chip that comprises one or more holes as ion transport measuring means, the chip can be baked, and subsequently incubated in a base solution and then rinse in water or a dilution of PBS. In another example, after manufacture of a glass chip that comprises one or more holes as ion transport measuring means, the chip can optionally be baked, subsequently incubated in an acid solution, rinsed in water, incubated in a base solution, and finally rinsed in water or a dilution of PBS. In some preferred embodiments, the surfaces of a chip surrounding ion transport measuring means can be laser polished prior to treating the chip with acid and base.

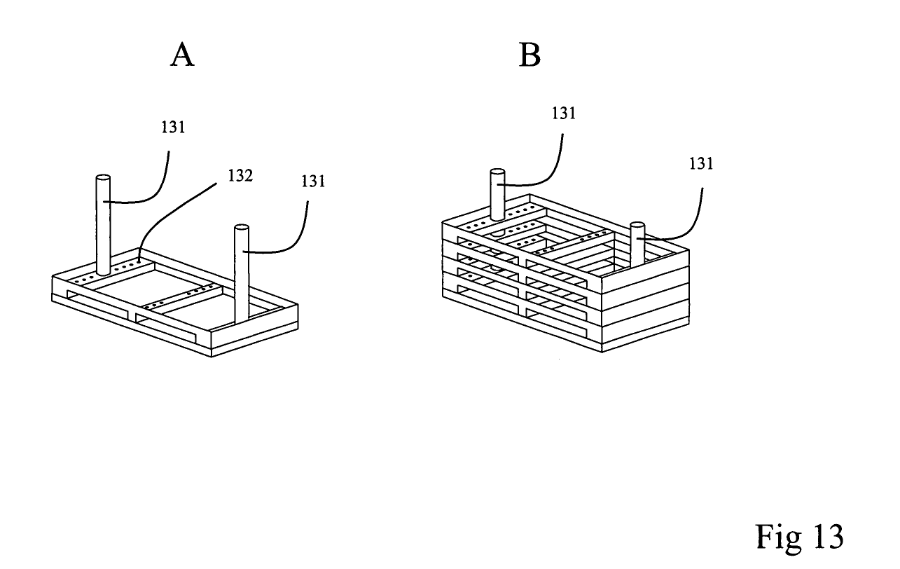

To facilitate batch treatment of glass biochips, we have used the treatment fixtures illustrated in FIG. 13. FIG. 13A shows a single layer treatment fixture that can fit into a glass jar containing acid, base, or other chemical solutions. The rods (131) facilitate handling and stacking of the treatment fixtures. Glass pins can fit into the holes (132) and chips can be stacked lengthwise on their edges between the pins. FIG. 13B shows the stacked treatment fixture. The fixture is made of acid and base resistant materials such as cyclo olefin polymers (for example, ZEONOR®), polyphenylene ether/PPO or modified polyphenylene oxide (for example, NORYL®), polytetrafluoroethylene, TEFLON™, etc. Multiple layers of these racks can be stacked up to fit into one glass container, as shown in FIG. 13B. This design also allows mechanisms of moving fluid to occur such as that brought about by a rotary or reciprocal shaker or a magnetic stir bar.

In an alternative design, chips are positioned flat on a treatment fixture, and are held in a tray by a door that can open and latch closed. This facilitates manipulation of the chips, such as by a machine. For example, after treatment of the chips, a machine that assembles cartridges can pick up a treated chip from the treatment fixture in order to attach it to a cartridge.

In some aspects of the present invention, it can be preferable to store an ion transport measuring means that has been treated to have enhanced sealing capacity in an environment having decreased carbon dioxide relative to the ambient environment. This can preserve the enhanced electrical sealing properties of the ion transport measuring means. Such an environment can be, for example, water, a salt solution (including a buffered salt solution), acetone, a vacuum, or in the presence of one or more drying agents or desicants (for example, silica gel, CaCl₂ or NaOH) or under nitrogen or an inert gas. Where an ion transport measuring means or structure comprising an ion transport measuring means is stored in water or an aqueous solution, preferably the pH of the water or solution is greater than 4, more preferably greater than about 6, and more preferably yet greater than about 7. For example, an ion transport measuring means or a structure comprising an ion transport measuring means can be stored in a solution having a pH of approximately 8.

Glass chips that have been base treated and stored in water with neutral pH levels can maintain their enhanced sealability for as long as 10 months or longer. In addition, patch clamp chips bonded to plastic cartridges via adhesives such as UV-acrylic or UV-epoxy glues can be stored in neutral pH water for months without affecting the sealing properties.

We have also tested patch clamp biochips and cartridges that were stored in a dry environment with dessicant for 30 days. The chips were re-hydrated and tested for sealing. In one experiment, we got 6/7 seals for the dry-stored chips. Similarly, we stored mounted chips in dry environment and were able to obtain seals after a few weeks of storage.

Dehydration can, however, reduce the sealability of chemically treated chips. To improve the seal rate for dry-stored chips, NaOH, NaCl, CaCl₂ and other salt or basic solutions can be used to rejuvenate the chips out of dry storage to restore the sealability.

The present invention also includes methods of shipping or transporting ion transport measuring means modified by the methods of the present invention to have enhanced electric sealing properties and structures comprising ion transport means that have been modified using the methods of the present invention to have enhance electric sealing properties. Such ion transport measuring means and structures comprising ion transport measuring means can be shipped or transported in closed containers that maintain the ion transport measuring means in conditions of low CO₂ or air. For example, the ion transport measuring means can be submerged in water, acetone, alcohol, buffered solutions, salt solutions, or under nitrogen (N₂) or inert gases (e.g., argon). Where the ion transport measuring means or structure comprising an ion transport measuring means is stored in water or an aqueous solution, preferably the pH of the water or solution is greater than 4, more preferably greater than about 6, and more preferably yet greater than about 7. For example, an ion transport measuring means or a structure comprising an ion transport measuring means can be shipped in a solution having a pH of approximately 8.

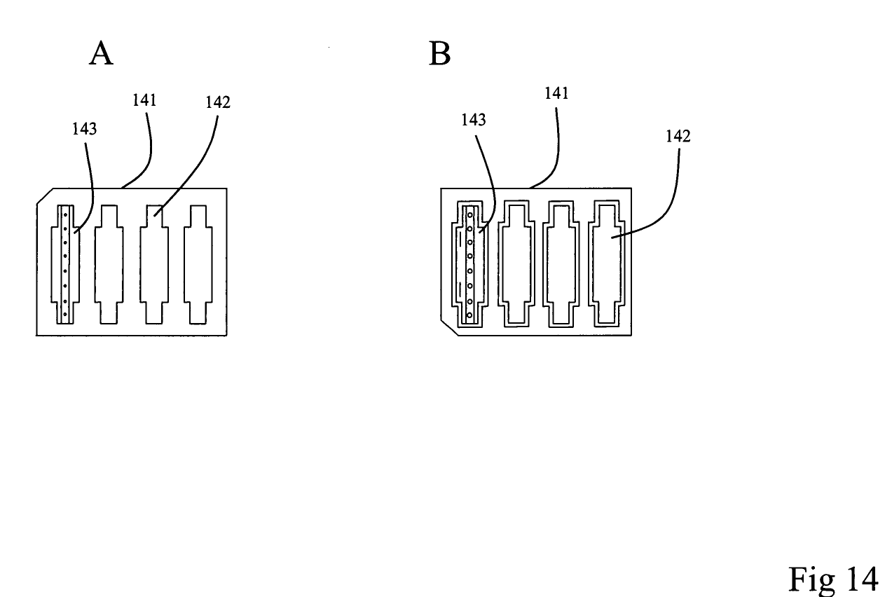

In one method of shipping a chip that has been treated to have enhanced sealing properties, the ion transport measuring devices comprising base-treated chips are shipped such that the chips are loaded up side down. The package for commercial shipments is designed to hold cartridges up side down, although the up side up configuration can also be used for shipping. To allow easy opening and facilitate automation in sequential loading of the devices onto apparatuses for use, a blister pack with film sealing is designed. As illustrated in the FIG. 14, a blister pack is provided in the form of a molded plastic frame (141) having (142) for positioning cartridges. One of the slots comprises a cartridge (143), viewed from the bottom in FIG. 14A and from the top in FIG. 14B. The blister pack has an opening on both top and bottom sides for film sealing. The sealing film or “lidstock” is a thin foil with temperature activated adhesive and an inert coating such as EVA (ethyl vinyl acetate) polymer. For wet (water) storage, the blister pack is first sealed from top (the opening side, flipped over, and the cartridges are loaded up side up. A preservative solution such as water is then injected into each well and the rest of the open space in each chamber of the package. Another lidstock film is then used to seal the blister package from the bottom. The blister package can be optionally sterilized with radiation for long shelf life.

Yet another aspect is related to the shipping of laser processed glass chips as finished goods between to production processes, particularly if the two processes are in different production locations. The current invention includes a shipping fixture allowing individual placement and securing of laser-processed glass chips for shipment. The same fixture-chips assembly is then directly used for subsequent chemical processing. To withstand strong acid and base treatment, the shipping fixtures are molded with inert materials such as polyphenylene ether/or modified polyphenylene oxide (e.g., Noryl®), Teflon, and cylco olefin polymers (e.g., Zeonor®). A stack of these fixtures can be secured in one container for chemical treatments, or for shipping in aqueous solutions such as water. The liquid shipping provides buffering for vibrations during transportation, giving maximum protection of glass chips from being damaged.

The present invention also includes ion transport measuring means treated to have enhanced electrical sealing properties, such as by methods disclosed herein. The ion transport measuring means can be any ion transport measuring means, including those disclosed herein. The present invention also includes chips, pipettes, substrates, and cartridges, including those disclosed herein, comprising ion transport measuring means treated using the methods of the present invention to have enhanced electrical sealing properties.

The present invention also includes methods of using ion transport measuring means and structures comprising ion transport measuring means, such as biochips, to measure ion transport activity or functions of one or more particles, such as cells. The methods include: contacting a sample comprising at least one particle with an ion transport measuring means that has been modified to enhance the electrical seal of a particle or membrane with the ion transport measuring means, engaging at least one particle or at least one membrane on or at the modified ion transport measuring means, and measuring at least one ion transport function or property of the particle or membrane. The methods can be practices using the methods and devises disclosed herein. Generally, the methods of the present invention provide the following characteristics, but not all such characteristics are required such that some characteristics can be removed and others optionally added: 1) the introduction of particles into a chamber that includes a biochip of the present invention, 2) optionally positioning particles at or near an ion transport detection structure, 3) electronic sealing of the particle with the ion transport detection structure, and 4) performing ion transport recording. Methods known in the art and disclosed herein can be performed to measure ion transport functions and properties using modified ion transport measuring means of the present invention, such as surface-modified capillaries, pipette, and holes and apertures on biochips and channel structures.

V. Methods for Measuring the Surface Energy of the Surface of a Chemically Treated Ion Tranport Measuring Biochip

Another aspect of the current invention originated from the need for an inexpensive, fast, and sensitive method to measure surface energy on solid/liquid surface such as, for example, that of a chemically treated ion transport measurement biochip.

The method includes: dispensing a drop of defined volume of water or an aqueous solution on a surface, measuring the time it takes for the drop to evaporate; and estimating the relative or absolute surface energy of the surface based on the evaporation time and the difference in evaporation time with respect to control samples.

The contact angle of a liquid drop on a solid surface is a measure of the surface energy, assuming constant liquid/air surface energy. Very low liquid/solid energy results in extremely small contact angles (close to 0 degrees). For that reason, contact angle measurements might not be a very sensitive method for low surface energy systems.

When a liquid drop with fixed volume is in contact with a solid surface, the air/liquid surface of the drop will be inversely proportional to the liquid/solid surface energy. Lower liquid/solid surface energy will result in bigger spreading of the drop. The evaporation of the drop will be proportional to the air/liquid surface area at any given moment. Thus the evaporation time will be proportional to the liquid/solid surface energy.

The method can be used to determine the hydrophilicity of any type of surface. For example, the method can be used to determine the hydrophilicity of at least a portion of the surface of an ion transport measuring chip. In this case, a drop of water or aqueous solution is dispensed on the surface of a biochip comprising at least one ion transport measuring means, preferably a biochip that has been chemically treated to improve its electrical sealing properties. Controls can be performed simultaneously with the hydrophilicity test, or can be performed at another time. Preferably, a range of controls are performed on surfaces of known hydrophilicity to provide a hydrophilicity scale. Evaporation of the drop is monitored, and the time elapsed between the time the drop contacts the chip and the time it has totally evaporated is measured. Preferably, the evaporation time of the test drop is compared with the evaporation times of the one or more controls, which can be expressed as a scale. The elapsed time is used as an index for hydrophilicity. This index can be used to determine whether a chemically treated chip is within the optimal range for achieving high resistance electrical seals.

Evaporation can be monitored by diffraction, reflectance, or interference at the surface where the drop is deposited, or simply by visual observation. Evaporation can also be monitored by measuring the change in intensity or other physical or chemical properties of a dye or tracer agent that has been used to color or label the solution.

The method is not limited to testing of biochips, but can be used to measure the hydrophilicity of a surface used for any purpose. The invention uses the evaporation time of a liquid drop on a solid surface as a measure of the solid/liquid surface energy. The method has very low cost (an accurate liquid dispenser is the only equipment needed). It is also very fast and accurate for low surface energy systems.

Using the drop evaporation technique, we have demonstrated that the evaporation time of a 0.25 microliter water drop is 2.5 times shorter for a highly hydrophilic glass surface (treated with base) compared to chemically untreated glass.

VI. Methods of Manufacturing Chips for Ion Transport Measurement Devices

Yet another aspect of the present invention is a method of making a chip for ion transport measurement devices by fabricating a chip that comprises multiple rows of ion transport measuring holes and subsequently breaking the chip into discrete segments that comprise a subset of the total number of ion transport measuring holes.

In this method, a glass sheet is pre-processed with a laser to create patch clamp recording apertures, and preferably treated chemically to improve sealability as described in this application. The glass sheet has also been pre-scored with a laser to produce mark lines by which sets of holes can be separated from one another. Preferably, the mark lines are continuous slashes that go through the glass to a depth of about 30% or more of the thickness of the sheet.

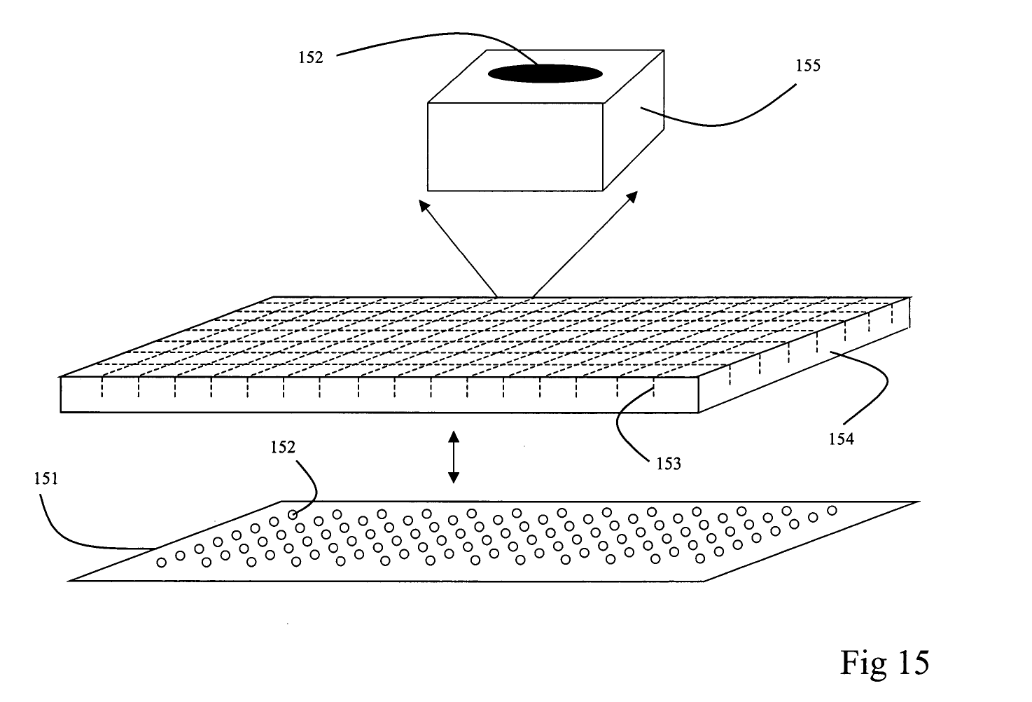

In some preferred embodiments, an injection molded multi-unit well plate is bonded to the glass with adhesives so that each well of the plate is in register with one of the ion transport recording holes. Sections of the multi-unit welled sheet sheet comprising a portion of the multi-unit well plate and a portion of the glass chip can be separated later by two metal plates closing in from two sides of the scored mark lines against the glass sheet, followed by bending of the bonded multi-well devices along with the metal plates and pulling of the segments away from each other. The severed sections can comprise one or more ion transport measuring units. FIG. 15 shows a glass chip (151) having ion transport measuring holes (152) and mark lines (153) created by a laser. The chip is attached to a multiwell plate that to form a multiunit sheet (154). Sections (155) that can comprise one or more ion transport measuring holes (152) can be detached from the sheet (154).

This approach allows for low cost, automated assembly of single well or low-density arrays, such as 16-well planar patch clamp consumables. This method of manufacture improves automation, and reduces individual unit assembly time.

VII. High Density Ion Transport Measurement Chips

Another aspect of the present invention is a high density, high throughput chip for ion transport measurement. A high density chip for ion transport measurement comprises multiple ion transport measuring holes. The invention also encompasses methods of making high-density consumable patch clamp arrays for ultra high throughput screening of ion transport function.

A high density chip for ion transport measurement comprises at least 24 ion transport measuring holes, preferably at least 48 ion transport measuring holes, and more preferably, at least 96 ion transport measuring holes. A high density, high throughput chip for ion transport measurement of the present invention can comprise at least 384 ion transport measuring holes, or at least 1536 ion transport measuring holes.

A high density ion transport measuring chip can be made using a silicon, glass, or silicon-on-insulator (SOI) wafer. The wafer is first wet-etched to create wells on the top surface, and then laser drilling is used to form the through-holes. The dimensions of the wafer and the wells can vary, however, in preferred embodiments in which a 1536 well array is fabricated, the thickness of the wafer can range from about 0.1 micron to 10 millimeters, preferably from about 0.5 micron to 2 millimeters, depending on the substrate.

For wafers in the range of 1 millimeter thick, the etching tolerance should be within 2% if the through-holes are approximately 30 microns in depth. This applies to silicon wafers etched with alkaline solutions such as KOH or glass wafers etched with buffered HF. With SOI wafers, a defined thickness of SiO₂ covers the Si wafers, and etching of the wells through the Si side with KOH will stop at the SiO₂ interface. This way the thickness of the remaining material is consistent across the whole wafer, and even consistent among different batches of etched wafers. This permits laser drilling on these etched substrates to be more standardized, and reduces the time needed for laser measurement. In a preferred embodiment, the etched Si wells have a volume of approximately 2 microliters, assuming a footprint of approximately 2 millimeters×2 millimeters for each well that extends as a prism or inverted pyramid shape through the Si substrate during anisotropic etching, leaving a distance of approximately 1 millimeter between adjacent wells.

In one design, the bottom of the chip can be sealed against a single common reservoir for measuring solution that is connected to a common reference electrode, while individual recording electrodes can be connected at the upper surface directly or via electrolyte bridges.

Alternatively, a structure with 1536 or any preferred number of individual isolated chambers can be sealed against the bottom of a 1536-well (or any preferred number of well) plate so that each chamber is in register with a well. In some designs of this embodiment, the top surface of the SOI wafer can be a common electrode, with the conductivity of Si material being adequate to provide electrical connection; however, additional metal coating on the top surface (applied before etching as mask layer) can increase conductivity of the upper surface. Wet etching that creates the wells removes this metal coating from the wells themselves. Chemical treatment with acid and/or base can optionally be performed on the chip for improved sealing.

Another way to make a high density chip is to use very thin wafers made of glass, SiO₂, quartz, Si, PDMS, plastics, polymers, or other materials, or a thin sheet, with thickness between about 1 micron and about 1 millimeter. Laser drilling can be performed on such sheets to create through-holes. A separate, “well plate” with 1536 or any preferred number of wells, manufactured by molding, etching, micro-machining or other processes, is then sealed against the holes via gluing or by using other bonding methods.

The laser drilling of the holes can be from the front or back side of the chip.For high density ion transport measuring chips, either a “standard” or inverted drilling configuration can be used as described herein.

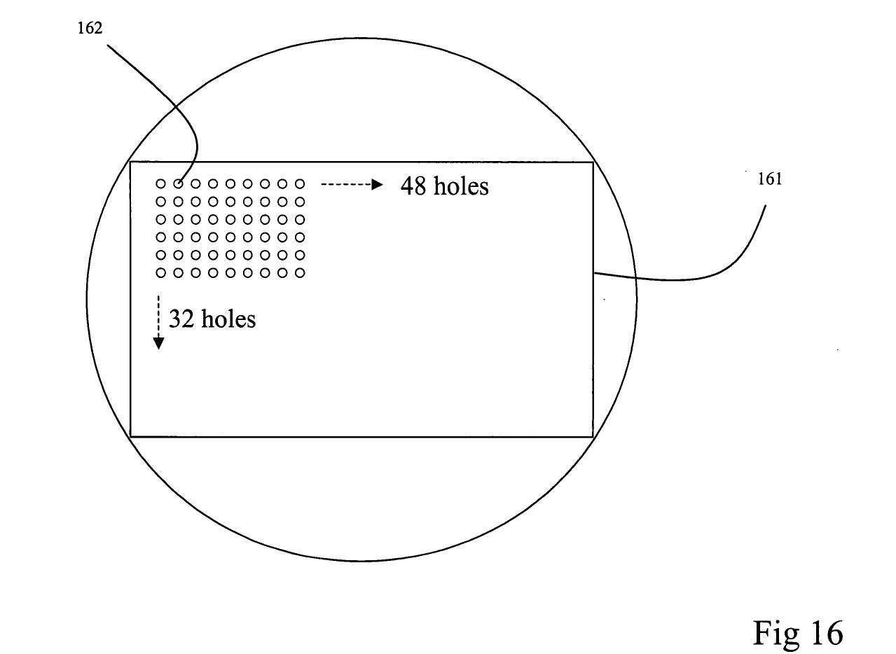

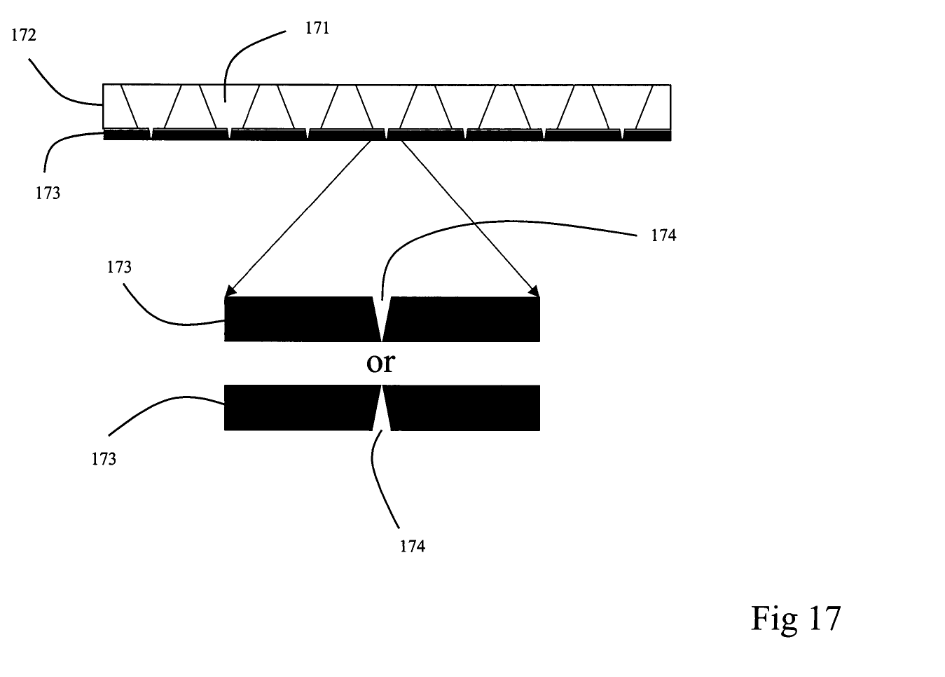

FIG. 16 shows a high density array made on a Si, glass, or SOI wafer (161). It is made with a wet etch process, which creates the wells (162) on the top surface, followed by laser drilling through the remaining of the material on the bottom of each of the wells. FIG. 17 shows the high density array having upper chambers (171) that can be formed by a well plate (172) attached to the chip (173). Wells (174) in the chip (173) having laser drilled through-holes can be oriented in inverted (top alternative) or standard (bottom alternative) orientation.

VIII. Methods for Assembling Ion Transport Measurement Cartridges

Use of Adhesives

A preferred embodiment of the present invention is an ion transport measurement device cartridge comprising one or more upper chamber pieces bonded via adhesive or other means to one or more ion transport measurement chips that have been treated to have enhanced electrical sealing properties in which the chip or chips contain at least one microfabricated ion transport measurement aperture (hole), optionally but preferably drilled by a laser. The one or more ion transport measurement chips are optionally laser polished on the side of the small exit hole, and treated with a combination of acid and base treatment as described herein.

The present invention also includes a method of assembling ion transport measurement cartridges by bonding the ion transport measurement chip(s) with an upper chamber piece. In one embodiment, an ion transport measurement chip containing one or more ion transport measuring apertures is bonded to an upper chamber piece via a UV-activated adhesive, such that each well of the upper chamber piece is in register with a recording aperture on the ion transport measurement chip, and the smaller, exit holes from laser drilling of the ion transport measuring holes are exposed to the wells of the upper chamber piece.

To facilitate efficient assembly, a registration bump can preferably be molded on the bottom of the upper chamber piece so that when the biochip is pressed against the bump and shoulder at the bottom of the upper chamber piece, the recording apertures on the ion channel measurement chip are in register with the wells of the upper chamber piece. An example of an upper chamber piece having alignment bumps (2) is shown in FIG. 1B.

Preferred UV adhesive include, but are not limited to, UV-epoxy, UV-acrylic, UV-silicone, and UV-PDMS.

The UV dose required to completely cure the UV adhesive can at times inactivate the treated surface of the chip. To avoid UV radiation to chip surface areas near the recording apertures where seals are to occur, a mask made of UV-permeate glass on which spots of size between 0.5 to 5 mm are provided by depositing a thin metal layer or paint (preferably a dark or black) layer.

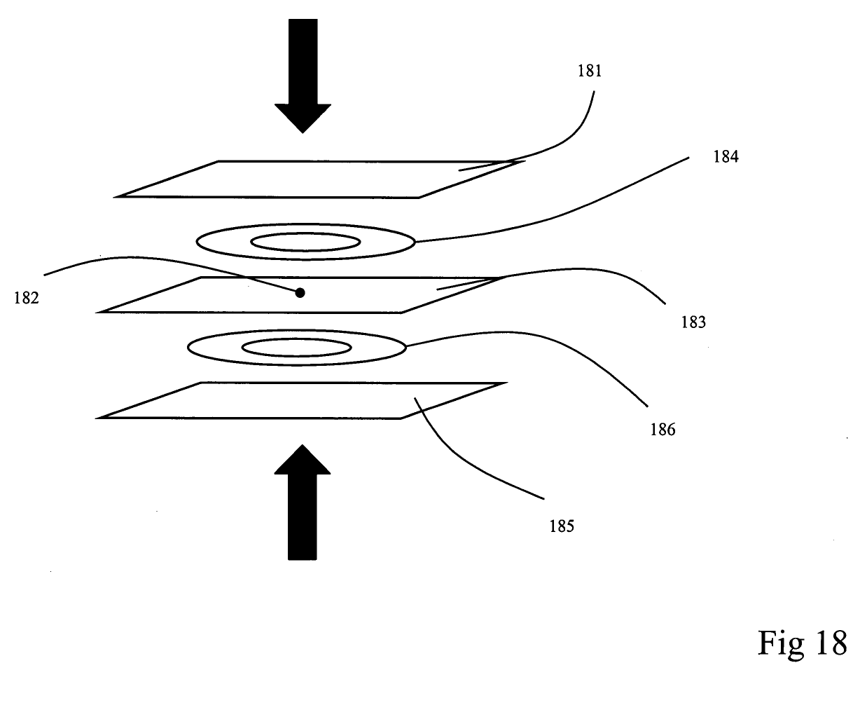

Pressure Mounting As an alternative to glue-based bonding, the upper chamber piece can be designed to allow an O-ring type of gasket made with PDMS to be used as seal cushion between the upper chamber piece and a biochip during a sandwich-type pressure mounting procedure. FIG. 18 depicts the general format for pressure bonding, in which a chip (183) is attached to an upper chamber piece (181) using a gasket (184) to form a seal between the upper chamber piece (181) and chip (183) when pressure (arrow) is applied. In this highly schematized depiction, a lower chamber piece (185) is also attached to the chip (183) using a second gasket (186) to form a seal between the lower chamber piece (185) and chip (183) when pressure (arrow) is applied. Mechanical pressure can be provided by a weight or clamp, or by any other means, including fasteners or holders.

IX. Biochip Device for Ion Transport Measurement Comprising Fluidic Channel Chambers

A further aspect of the present invention is a flow-through fluidic channel ion transport measuring device that can be part of a fully automated ion transport measuring device and apparatus. This device comprises a planar chip that comprises ion transport measuring holes, and upper and lower chambers on either side of the chip that are fluidic channels. One or more fluidic channels is positioned above the chip and one or more fluid channels is positioned below the chip. Apertures are positioned in the fluidic channels such that an ion transport measuring hole in the chip has access to an upper fluidic channel (serving as an upper chamber) and a lower fluidic channel (serving as a lower chamber).

A chip of a fluidic channel ion transport measuring device can have multiple ion transport measuring holes, and each of the holes can be in fluid communication with an upper fluidic channel and a lower fluidic channel. The upper fluidic channel or channels can be connected with one another, and more than one lower fluidic channel can be independent; or the device can have two or more upper fluidic channels that can be independent while the one or more lower fluidic channels can be connected with one another. In a yet another alternative, upper fluidic channels that service different ion transport measuring holes can be separate from one another and the lower fluidic channels that service different ion transport measuring holes can also be separate from one another.

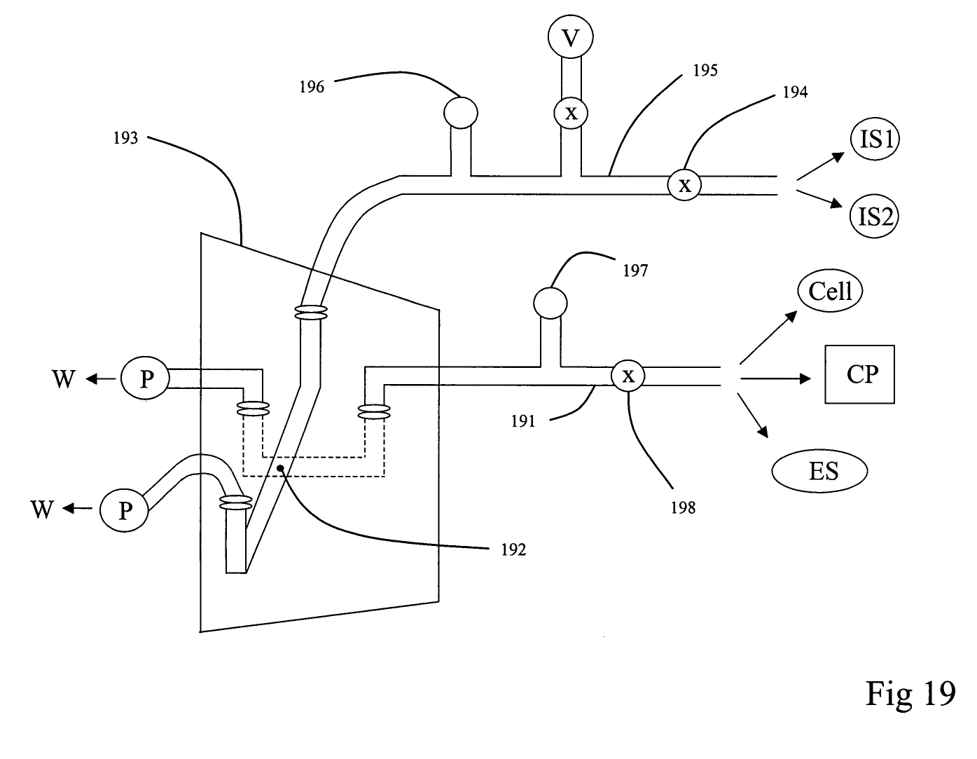

FIG. 19, depicts a schematic view of one possible design of a planar patch clamping chip (193) having an upper fluid channel (191) for extracellular solution (ES) and a lower fluidic channel (195) for intracellular solutions (IS1, IS2). The upper and lower channels are interfaced at a point where the recording aperture (192) of the planar electrode resides. Separate fluidic pumps (P) drive the flow of fluids through the two (upper and lower) fluidic channels. Recording (196) and reference electrodes (197) external to the fluidic patch clamp chip are connected via an electrolyte solution bridge to the upper (191) and lower (195) fluidic channels. A pressure source such as a pump with pressure controller that can generate both positive and negative pressures is shown linked to the lower fluidic channels. A multi-way valve (194) can be used to connect the lower fluidic channel (195) to different solution reservoirs (IS1, IS2, etc), and a multi-way valve (198) can be used to connect the upper fluidic channel (191) to cell reservoirs, a compound plate (CP), wash buffers, or other solutions.

In some preferred aspects, the device can have a molded upper piece that comprises one or more upper channels, and a molded lower piece that comprises one or more lower channels. The channels can be drilled through or molded into the pieces, which preferably comprises at least one plastic. A chip comprising one or preferably, multiple ion transport measuring holes can be situated between the upper piece and the lower piece, such that an ion transport measuring hole through the chip connects an upper channel of the upper piece with a lower channel of the lower piece.

In some preferred embodiments of these aspects, an upper conduit connects to a well that is in register with a hole of the chip. In addition to being accessed by the conduit, the well can be open at the top, for the addition of, for example, cell suspensions or compounds. Preferably, these preferred embodiments, the chip comprises multiple holes and the upper piece comprises multiple wells in register with the holes of the chip. Preferably, each well is accessed by a separated and independent channel. The lower piece can comprise one or more lower channels. Preferably, in these embodiments, the lower piece comprises at least one channel, and each of the at least one channel accesses two or more ion transport measuring holes in the biochip. The at least one lower channel can comprise or be in electrical contact with an electrode, such as, for example, a reference electrode. Upper chamber electrodes can be dunked into well from above, inserted into the upper channels, or otherwise brought into electrical contact with the upper wells.

Designs comprising upper chamber fluidic channels, lower chamber fluidic channels, or both upper and lower chamber fluidic channels have several advantages. The external electrodes can be of multiple use, but replaceable. This reduces the cost of the biochip. The flow-through fluidics of both the upper and lower chambers minimizes the generation of air bubbles. Importantly, the closed fluidic channels allow for controlled delivery of low volume fluids without evaporation.

X. Methods of Preparing Cells for Ion Tranport Measurement

In a further aspect of the present invention, methods for isolating attached cells for planar patch clamp electrophysiology are provided. Conventional cell isolation methods by non-enzymatic, trypsin, or reagent-based methods will not produce cells that are in optimal condition for high throughput electrophysiology. Typically cells produced by available protocols are either over-digested and tend to function less than optimally in planar patch clamp studies, or under-digested and resulting in cell clumps with the cell suspension. In addition, the cells isolated by conventional methods tend to have large amounts of debris which are a major source of contamination at the recording aperture. The current protocols are optimized for better cell health, single cell suspension, less debris and good patch clamp performance. The current protocols can be used to isolate cells for any purpose, particularly when cells in an optimal state of health and integrity are desirable, including purposes that are not related to electrophysiology studies.

This invention was developed to produce suspension CHO and HEK cells that give high quality patch clamp recording when used with chips and devices of the present invention. Parameters such as cell health, seal rate, Rm (membrane resistance), Ra (access resistance), stable whole cell access, and current density, were among the parameters optimized. The method includes: providing a population of attached cells, releasing the attached cells using a divalent cation solution, an enzyme-containing solution, or a combination thereof; washing the cells with a buffered cell-compatible salt solution; and filtering the cells to produce suspension cells that give high quality patch clamp recordings using ion transport measuring chips.

Enzyme-Free Cell Preparation

Enzyme-free dissociation is desirable when an ion transport expressed on a cell surface can be digested by enzymatic methods, thereby causing a change in ion transport properties. Enzyme-free methods involve a dissociation buffer that is either Ca⁺⁺-chelator-based or non-Ca⁺⁺-chelator-based. The former is typically a solution of EDTA, while the latter can be calcium-free PBS. In such methods, attached cells grown on plates are first washed with calcium-free PBS, and then incubated with the dissociation buffer. In case of the calcium chelator-based dissociation, the dissociated cells must be washed at least once with a chelator-free solution before they can be used for ion transport measurement assays. The suspended cells are then passed through a filter, such as a filter having a pore size of from about 15 to 30 microns (this can vary depending on the type of cells and their average size).

Preparation of Cells Using Enzyme

In some methods (see Example 6), trypsin is used to dissociate attached cells. In such methods, the cells are typically rinsed with a solution devoid of divalent cations, and then briefly treated with trypsin. The trypsin digestion is stopped with a quench medium carefully designed to achieve the optimal divalent cation mix and concentration. In the methods provided herein, the suspended cells are then passed through a filter, such as a filter having a pore size of from about 15 to 30 microns (this can vary depending on the type of cells and their average size).

Another enzyme-based method uses a preparation commercially available from Innovative Cell Technologies (San Diego). Accumax is an enzyme mix containing protease, collagenase, and DNAse. Example 6 provides a protocol for CHO cells using Accumax and filtration.

Some preferred methods of the present invention use a combination of enzyme-free dissociation buffer, Accumax reagent, and filtration to isolate high quality cells for patch clamping (see Example 6).

XI. Pressure Control Profile Protocol for Ion Transport Measurement

The present invention also provides a pressure protocol control program logic that can be used by an apparatus for ion transport measurement to achieve a high-resistance electrical seal between a cell or particle and an ion transport measuring means on a chip of the present invention in a fully automated fashion. In this aspect, the program interfaces with a machine that can receive input from an apparatus and direct the apparatus to perform certain functions.

Typically it has required months to years of experience on the part of an experimenter to master the techniques required to achieve and maintain high quality seals during their experiments. It is an object of the invention to produce a pressure protocol for achieving and maintaining seal quality parameters for automated patch clamp systems. The present invention provides a logic that can direct mechanical and automated patch clamp sealing of particles and membranes.

The program logic includes: a protocol for providing feedback control of pressure applied to an ion transport measuring means of an ion transport measuring apparatus, comprising: steps that direct the production of positive pressure; steps that direct the production of negative pressure; steps that direct the sensing of pressure; and steps that direct the application of negative pressure in response to sensed pressure in the form of multiple multi-layer if-then and loop logic, in which the positive and negative pressure produced is generated through tubing that is in fluid communication with an ion transport measuring means of an apparatus, and in which negative pressure is sensed through tubing that is in fluid communication with an ion transport measuring means of an apparatus. Preferably, these steps are performed in a defined order that depends on the feedback the apparatus receives. Thus, the order of steps of the protocol can vary according to a defined script depending on whether a seal between a particle and the ion transport measuring means is achieved during the operation of the program, and the properties of the seal achieved.

An apparatus for ion transport measurement that is controlled at least in part by the pressure program preferably comprises: at least one ion transport measurement device comprising two or more ion transport units (each comprising at least a portion of a biochip that has an ion transport measuring means, at least a portion of an upper chamber, and at least a portion of a lower chamber, and is in electrical contact with at least one recording electrode and at least one reference electrode), tubing that connects to the device and is in fluid communication with the two or more ion transport measuring means of an apparatus, and pumps or other means for producing pressure through the tubing. Preferably, the apparatus is fully automated, and comprises means for delivering cells to upper chambers (such means can comprise tubing, syringe-type injection pumps, fluid transfer devices such as one or more automated fluid dispensors) and means for delivering solutions to lower chambers (such means can comprise tubing, syringe-type injection pumps).

Preferably, in addition to promoting and maintaining a high resistance seal, the pressure protocol program can also direct the rupture of a cell or membrane delineated particle that is sealed to an ion transport measuring means. Such rupture can be by the application of pressure after sealing, and can be used to achieve whole cell access.

In operation, the program directs the apparatus to generate a positive pressure in the range of 50 torr to 2000 torr, preferably between 500 and 1000 torr, to purge any blockage of the recording holes. Then the program directs the apparatus to generate a positive holding pressure between 0.1 to 50 torr, preferably between 1 to 20 torr to keep the recording aperture of an ion transport measuring chip clear of debris during the addition of cells to the upper chamber. After cell addition, the program directs the release of pressure and holds the pressure at null long enough to allow cells to approximate the aperture. The program then directs a negative pressure to be applied draw a cell onto (and partly into) the ion transport recording aperture for landing and the formation of a gigaohm seal. Additional pressure steps as described Example 7 may be required for achieving gigaohm seals if a seal does not occur upon cell landing.

To achieve whole-cell access, negative pressure is increased in progressive steps until the electrical parameters indicate the achievement of whole-cell access. Alternatively, the program can direct the application of a negative pressure to a “sealed” cell that is insufficient to gain whole-cell access, and then use a electric “zap” method to disrupt the membrane patch within the aperture and thereby achieve whole-cell access. Upon achieving whole-cell access the pressure is either released immediately, or held for a few seconds then released, depending on the cell quality. Finally, during whole-cell access procedures, the seal quality could be improved after access is achieved, then held at optimal parameters by a more complex pressure protocol.

The pressure protocol involves many branchpoints or “decisions” based upon feedback from the seal parameters. It is easiest to describe the protocol as a series of steps in programming logic, or program. A pseudocode example of such logic is provided as Example 7.

The program, also herein referred to as program logic, control logic or programming logic, can be illustrated and described in different manners. The procedures and processes described in this program herein are one possible embodiment of the program. Decision branches, loops, and other components can be performed in substantially different methods to obtain the same or substantially similar results, such as the use of an “if-then” loop in place of a “while” loop. The exemplary pseudocode and program description contained herein is not intended to be limiting, merely they are examples of one possible embodiment of encoding this program. One skilled in the art will realize that the procedures and processes of this program can be accomplished in a number of programming and encoding methods, on devices such as personal computers, chipsets, mainframe computers, and other electronic devices capable of performing and executing programmed code. Additionally, the steps described herein may be executed and performed in other step-wise processes to achieve the same or substantially similar results.

The procedures and descriptions of this program are described and illustrated across several pages. Some procedures are illustrated across several figures. This is not intended to limit the varied calculations and functions of these procedures to sub-routines separated from the rest of the procedure, instead it is a result of space limitations in the drawing of the figures. Certain aspects illustrated across several figures are intended to be connected seamlessly, and operate together as one procedure or subroutine. Off-page and on-page connectors are utilized to illustrate this continuity, and are not intended to confine the execution of certain code to specific areas of the illustrated figures. These illustrative connectors are additionally not intended to be additional steps in the execution of the program disclosed herein.

The program disclosed herein can be run and executed on a variety of systems. The program can be run on a device such as SealChip™ from Aviva Biosciences Corporation, the PatchXpress™ from Axon Instruments, or any other electronic patch-clamp system, as described in this present application or known in the art.

Additionally, the present invention can be executed in a computer-based manner. The computer-based manner of the present invention includes computer hardware and software. The computer-based program can run on a personal computer of the traditional type, including a motherboard. The motherboard contains a central processing unit (CPU), a basic input/output system (BIOS), one or more RAM memory devices and ROM memory devices, mass storage interfaces which connect to magnetic or optical storage devices including hard disk storage and one or more floppy drives, and may include serial ports, parallel ports, and USB ports, and expansion slots. The computer is operatively connected by wires to a display monitor, a printer, a keyboard, and a mouse, though a variety of connection means and input and output devices may be substituted without departing from the invention. Additionally, the present invention can be encoded on a chipset, or be encoded on computer-like components included in other devices.

A computer used in connection with the computer program may run an IBM-compatible personal computer, running a variety of operating systems including MS-DOS®, Microsoft® Windows®, or Linux®. Alternatively, the computer program may run on other computer environments, including mainframe systems such as UNIX® and VMS®, or the Apple® personal computer environment, portable computers such as palmtops, programmable controllers, or any other digital signal processors.

All of these elements and the manner in which they are connected are well-known in the art. In addition, one skilled in the art will recognize that these elements need not be connected in a single unit such as personal computer or mainframe, but may be connected over a network or via telecommunications links. The computer hardware described above may operate as a stand-alone system, or may be part of a local area network, or may comprise a series of terminals connected to a central system. Additionally, some or all aspects of the logic of the present invention can be encoded to run on a chipset or other electronic hardware. Additionally, the entire program may comprise a portion of a larger program wherein this section is called as part of the normal execution of the larger program, and all references to stopping or ending execution in this case refer to returning from this section of the program to the calling routine.

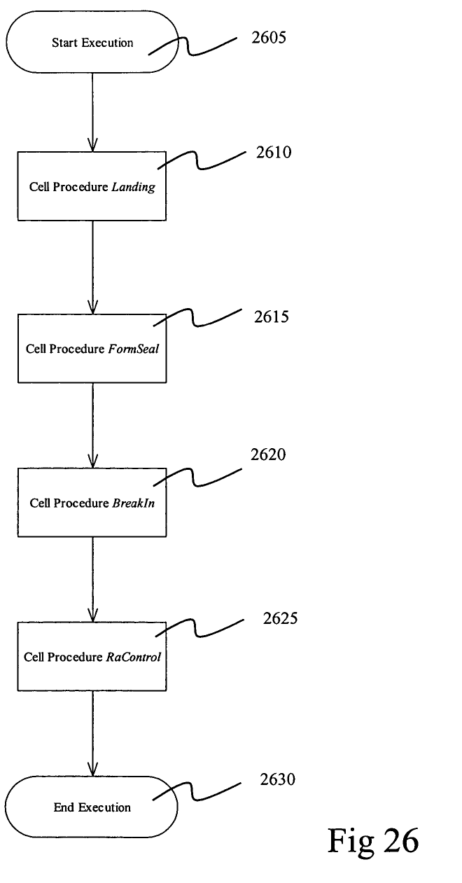

An overview of the program is disclosed in FIG. 26. The program comprises 4 separate procedures: Procedure Landing (2610), Procedure FormSeal (2615), Procedure BreakIn (2620), and Procedure RaControl (2625). The program starts (at step 2605) by being called from a separate controlling software or as a result of a user-initiated action. The program first runs the Procedure Landing (2610) to place a cell onto (and partly into) the ion transport recording aperture. When Procedure Landing (2610) has ended, the program runs Procedure FormSeal (2615) to form a gigaohm seal. Next the program calls Procedure BreakIn (2620) to achieve whole-cell access. The program then runs Procedure RaControl (2625). When completed, the control logic continues to step 2630 and ends. After the execution stops, a separate program will handle the application of voltage clamp protocols and the acquisition of data pertaining to ion channel activity. An unillustrated alternate mode of execution for this program will skip directly to Procedure RaControl (2625) to handle cells that have already been accessed but whose access resistance has increased beyond RaIdeal. This provides an opportunity to improve the quality of recordings in the middle of an experiment. Once a procedure called or run by the program ends, the program returns to run or execute the next procedure illustrated by FIG. 26. The individual procedures are described below.

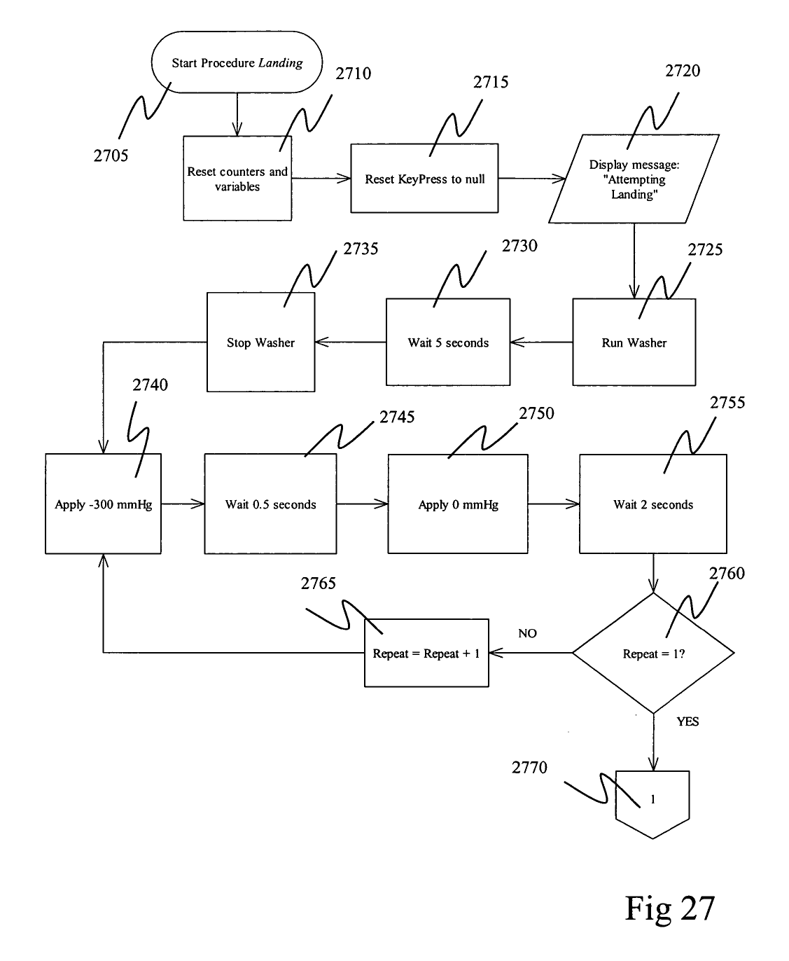

With reference to FIGS. 27, 28, and 29, Procedure Landing is now described. At step 2610, the program begins Procedure Landing. The start of Procedure Landing is identified by step 2705. All of the counters and variables used in the program are assigned and are reset (2710), then the variable KeyPress, which traps user input instructions, is set to null (2715). The program displays (2720), through a screen or other similar display device, the message “Attempting Landing” to indicate the progress of the control logic. Next, the program runs a Washer (2725), a pump-driven fluid delivery system, to rinse fluidics channels, which purges any blockage of the recording holes and clears any particles that may be present in the chambers before they have an opportunity to block the recording hole. The program waits 5 seconds (2730) while Washer is run, then the program stops the Washer (2735). The program then applies −300 torr of pressure (2740) to clear away any left-over bubbles, waits 0.5 seconds (2745), then applies 0 torr of pressure (2750). The control logic then waits 2 seconds (2755) for the measurements to stabilize. At step 2760, the program checks to see if the variable Repeat is equal to 1. If Repeat is not equal to 1, the program adds 1 to the value for Repeat (2765), and returns to step 2740. If at step 2760 the value of Repeat is 1, the control logic continues to step 2810 of Procedure Landing (as illustrated by off-page connector 2770 pointing to its matching off-page connector 2805).

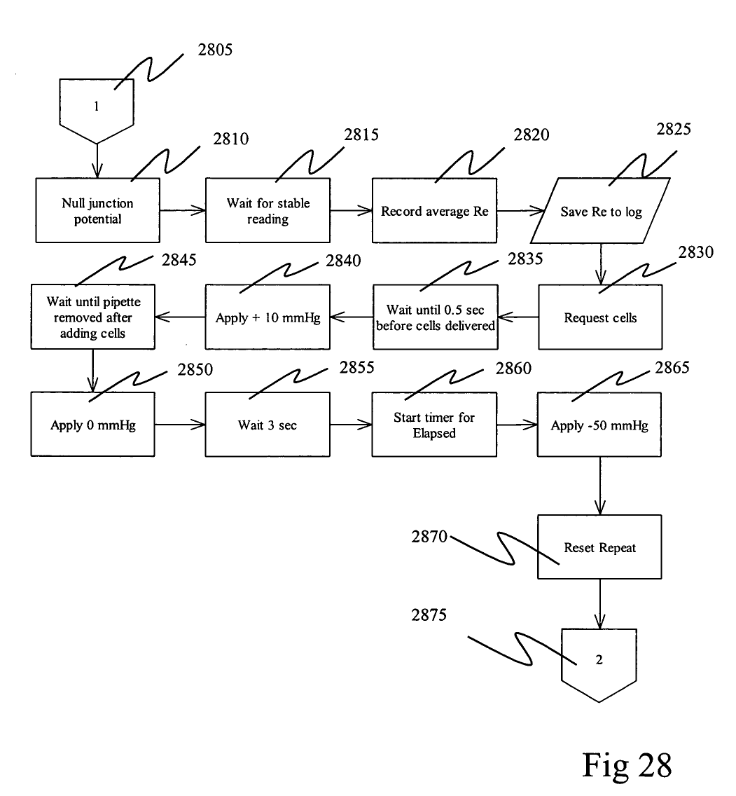

With reference to FIG. 28, Procedure Landing continues. The program next nulls the junction potential (2810), waits for a stable reading (2815), then records the average Re (2820), and saves the Re to logs in a file stored on the computer (2825). Next the program requests cells (2830)from a separate program or routine not listed here, and waits until 0.5 seconds before cells would be introduced to the recording chamber (2835). The program then applies +10 torr of pressure (2840) to keep the holes cleared during cell delivery, and then waits until the pipette has completed the cell delivery and is removed after adding cells (2845). The program then applies 0 torr (the units of torr and mmHg are interchangeable terms) of pressure (2850), waits 3 seconds (2855) to enable the cells to settle closer to the recording aperture. The program then starts a timer for Elapsed (2860), then applies −50 torr of pressure (2865) to attract a cell to the aperture. The control program then resets the Repeat variable to 0 (2870), and continues to step 2910 of Procedure Landing (as illustrated by off-page connector 2875 pointing to off-page connector 2905).

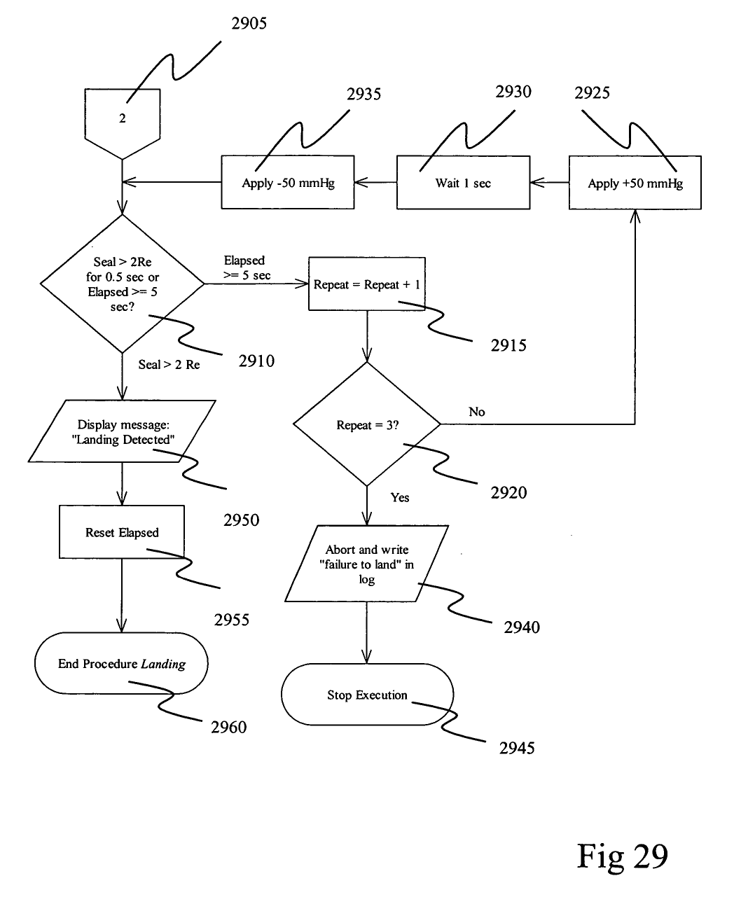

With reference to FIG. 29, Procedure Landing continues. The program then checks at step 2910 to see whether the Seal is greater than 2 ×Re for 0.5 seconds, or whether Elapsed time is greater than or equal to 5 seconds. If Elapsed time is greater than or equal to 5 seconds, the program then adds 1 to the value of stored variable Repeat (2915), then checks whether Repeat is equal to 3 (2920). If Repeat is not equal to 3, the program continues to step 2925 and applies +50 torr of pressure. The program waits 1 second (2930), then applies −50 torr of pressure (2935), then returns to step 2910. If at step 2920, the program determines that Repeat is equal to 3, the program continues to step 2940. The program aborts, records “failure to land” in its log, then ends the execution of the program (2945). At this point the chamber should be clean and prepared for removal.

If at step 2910 the program determines that Seal is greater than 2 33 Re, the program displays the message “Landing Detected” (2950), resets the value for Elapsed (2955), and ends Procedure Landing at step 2960. As illustrated by the program overview of FIG. 26, once Procedure Landing is run, the program next continues to step 2615 and runs Procedure FormSeal.

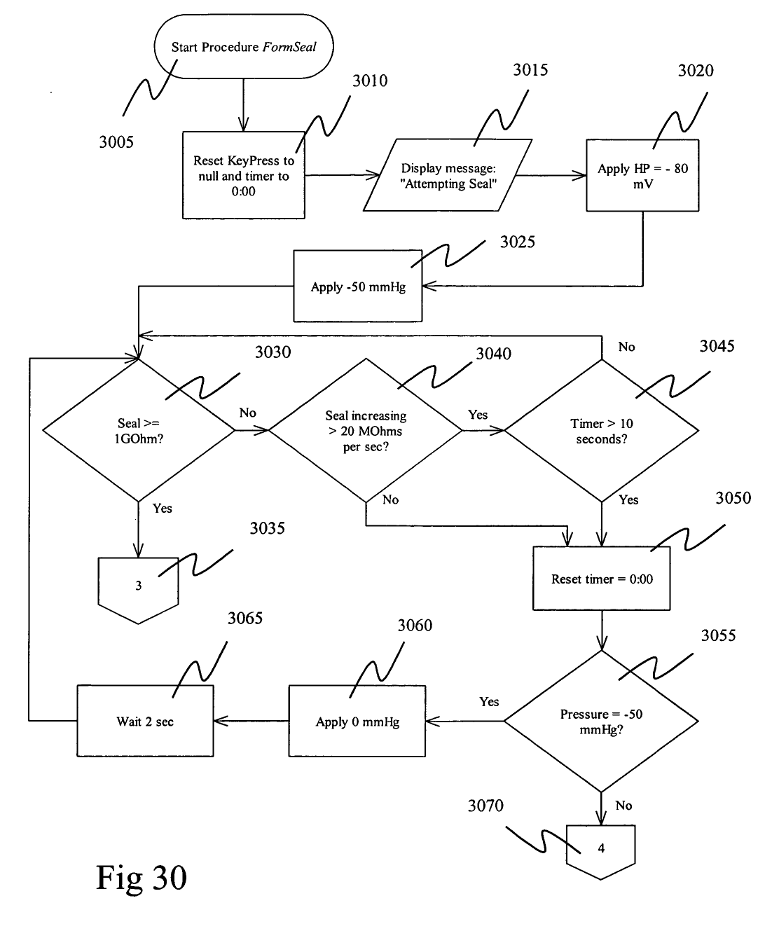

Procedure FormSeal is illustrated by FIGS. 30, 31, 32, and 33. The program calls Procedure FormSeal at step 2615. The start of Procedure FormSeal is illustrated by step 3005. The program resets KeyPress to null, and the timer to 0:00 (3010). As used throughout this program, when the variable Timer or Elapsed is reset, it immediately starts counting time in seconds. The program then displays the message “Attempting Seal” on an output device (3015). The program then applies a negative holding potential to the electrode immediately after landing by applying HP=−80 mV (3020). The program then applies −50 torr pressure (3025). At step 3030, the program checks whether the seal between the cell and the recording aperture presents greater than or equal to 1 one gigaOhm (a “gigaseal”) of resistance across the recoding aperture. If the seal is greater than or equal to 1 gigaOhm, the program proceeds to step 3310 of Procedure FormSeal (as illustrated by off-page connector 3035 pointing to off-page connector 3305). If at step 3030 the program determines that the seal is not greater than or equal to 1 gigaOhm, the program checks if the seal is increasing greater than 20 megaOhms per second (3040). If the seal is increasing greater than 20 megaOhms per second, the program continues to step 3045. If at step 3040 the program determines that the seal is not increasing greater than 20 megaOhms per second, then the program continues to step 3050. At step 3045, the program checks whether the timer has reached 10 seconds. If it has not, the program returns to step 3030. If at step 3045 the program determines that the timer is greater than 10 seconds, the program continues to step 3050.

At step 3050 the program resets the timer to 0:00, and checks whether the pressure is equal to −50 torr (3055). If pressure is −50 torr, the program applies 0 torr of pressure (3060), waits 2 seconds (3065), and returns to step 3030. If at step 3055 the program determines that pressure is not equal to −50 torr, the program continues with Procedure FormSeal (as illustrated by off-page connector 3070 pointing to off-page connector 3105). This section of the program ensures that a landing happens, and tests whether simple pressure steps are capable of producing a gigaOhm seal.

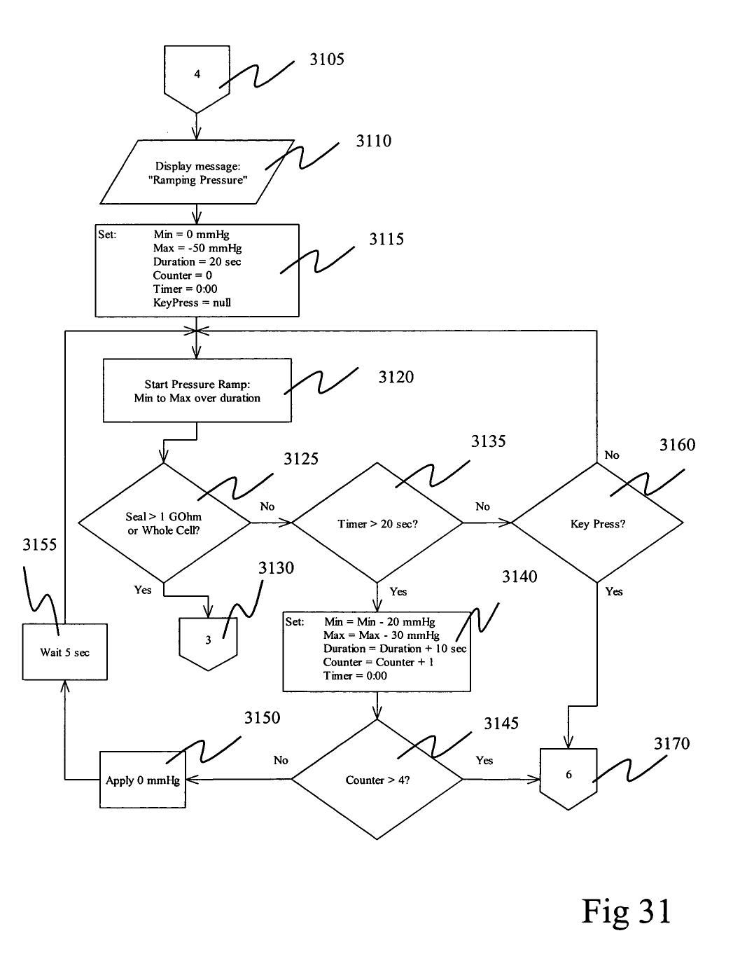

With reference to FIG. 31, Procedure FormSeal continues by displaying the status message “Ramping Pressure” (3110). The program then optimally assigns a set of values for variables to initially be used during the pressure ramp (3115). Min is set to 0 torr, Max is set to −50 torr, Duration is set to 20 seconds, Counter is set to 0, and Timer is set to 0:00. The program then executes a pressure ramp loop. Starting with step 3120, the program ramps the pressure from Min to Max over the Duration, using the assigned values for these variables. The program then checks to see if seal is greater than 1 gigaohm, or if “whole-cell access” has been achieved (3125). Whole-cell test is where capacitance is greater than 3.5 pF. If either of the conditions at step 3125 are true, the program continues with Procedure FormSeal at step 3310 (as illustrated by off-page connector 3130 pointing to off-page connector 3305).

If at step 3125 both of the conditions are false, the program moves to step 3135, where it checks whether Timer is greater than 20 seconds. If Timer is greater than 20 seconds, the program modifies the set of values for the variables used during the pressure ramp (3140). Min is reduced by 20 torr, Max is decreased by 30 torr, Duration is increased by 10 seconds, Counter is incremented by 1, and Timer is set to equal 0:00. The program checks whether Counter is greater than 4 (3145). If Counter is greater than 4, Procedure FormSeal continues to step 3210 (as illustrated by off-page connector 3170 pointing to off-page connector 3205). If Counter is less than 4, the program applies 0 torr of pressure (3150), waits 5 seconds (3155), then returns to the beginning of the pressure ramp loop that begins at step 3120.

If at step 3135 the program determines that Timer is not greater than 20 seconds, the program checks whether a user input key has been pressed (3160). If a key has been pressed, Procedure FormSeal continues with step 3205 (as illustrated by off-page connector 3170 pointing to off-page connector 3205). If at step 3160 a key has not been pressed, the program returns to the beginning of the pressure ramping loop that begins at step 3120.

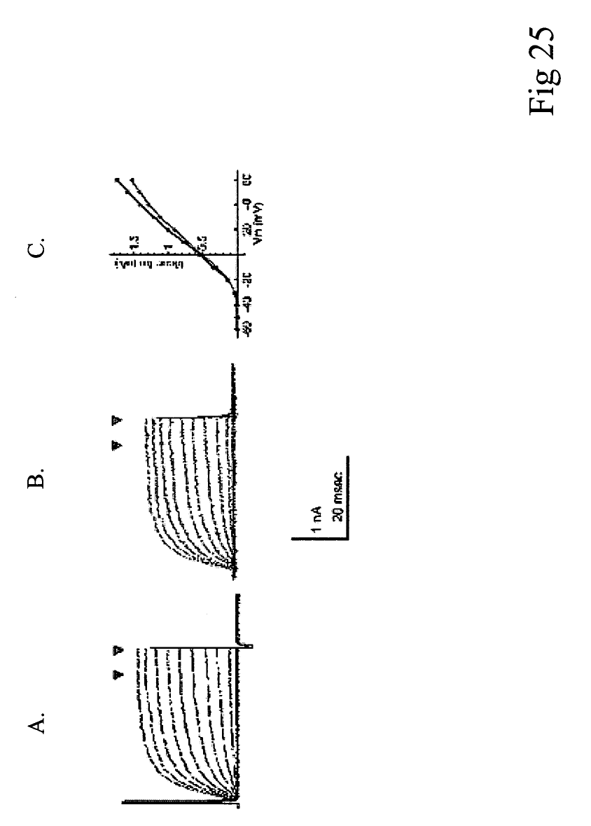

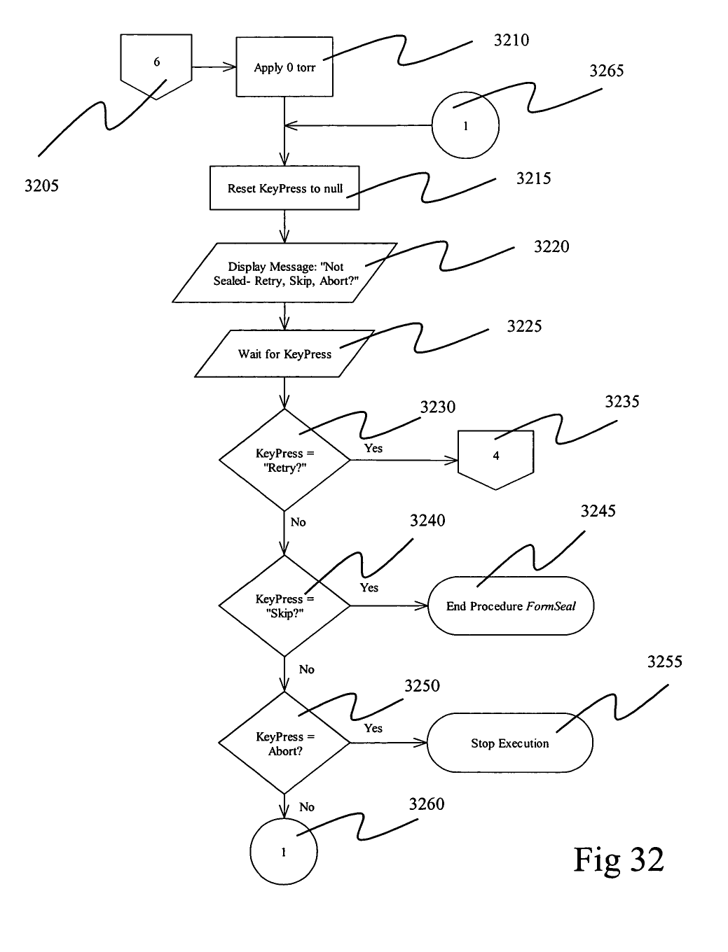

With reference to FIG. 32, Procedure FormSeal continues. At step 3210, 0 torr of pressure is applied. The program then resets the value to null whether a key has been pressed by the user (3215). The program then displays “Not sealed—Retry, Skip, Abort?” (3220). The program waits for the user to input whether to retry Procedure FormSeal, skip Procedure FormSeal, or abort the program altogether (3225). The program checks for input by the user. If the user enters “Retry” (3230), the program returns to step 3110 of Procedure FormSeal (as illustrated by off-page connector 3235 pointing to off-page connector 3105) to rerun the pressure ramp loop from its start. If the user inputs “Skip” (3240), the Procedure FormSeal ends (step 3245). Once Procedure FormSeal has run, as illustrated by the program overview of FIG. 25, the program next continues to step 2620 and runs Procedure BreakIn. If the user enters “Abort” (3250), the program stops executing and ends (3255). If no input has been received by step 3250, the program return to continue the input loop (as illustrated by connector 3260 pointing to connector 3265.

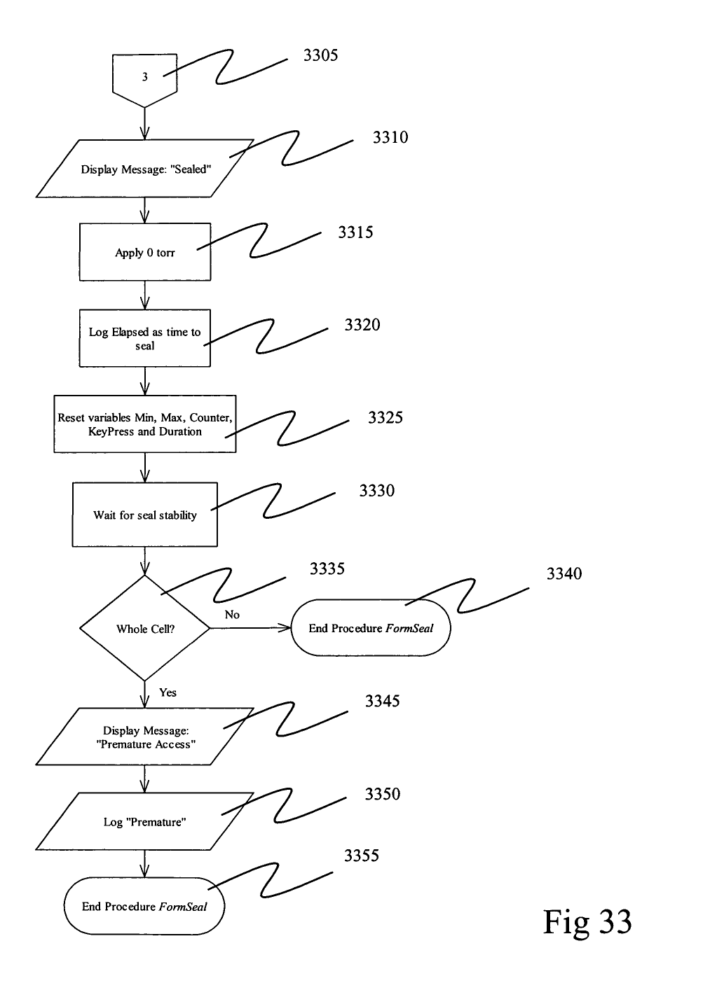

As illustrated by FIG. 33, Procedure FormSeal continues with step 3310 and displays the message “Sealed.” The program applies 0 torr pressure (3315), saves Elapsed time as time to seal in the logs (3320). The program then resets the values for Min, Max, Counter, KeyPress, and duration to null (3325). The program monitors the stability of the seal (3330), and continues once the seal is stable. If capacitance is not greater than 3.5 pF (“whole-cell”) (3335), Procedure FormSeal ends (3340), and as illustrated by the program overview of FIG. 26, the program next continues to step 2620 and runs Procedure BreakIn. If at step 3335 the program determines that capacitance is greater than 3.5 pF, the program displays “Premature Access” (3345), then writes this feature to the logs (3350) and Procedure FormSeal ends (3355). The program next continues to step 2620 and runs Procedure BreakIn.

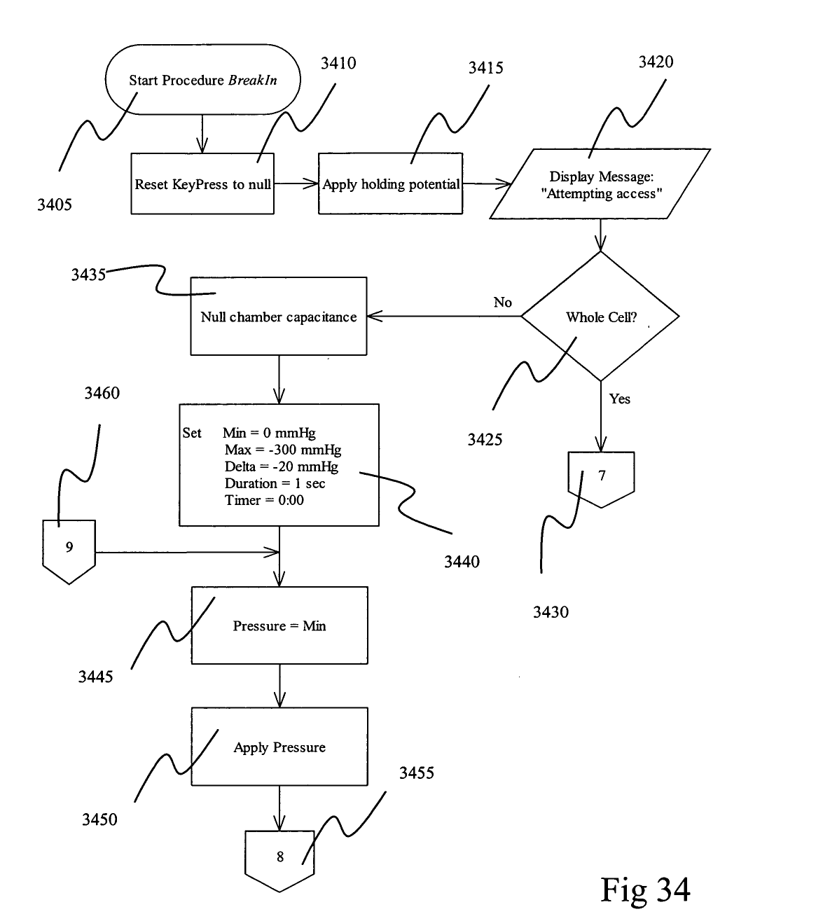

With reference to FIGS. 34, 35, 36, and 37, Procedure BreakIn is now described. The program runs Procedure BreakIn at step 2620. Procedure BreakIn starts, as illustrated by FIG. 34, at step 3405. The program resets the value for KeyPress to null (3410), then applies holding potential that is appropriate for the assay (3415). The program displays “Attempting access” (3420), then verifies whether whole-cell access has already been achieved (3425). If whole-cell has been achieved, Procedure BreakIn continues to step 3610 (as illustrated by off-page connector 3430 pointing to off-page connector 3605). If whole-cell has not been achieved at step 3425, the program nulls the chamber electrode capacitance (3435). The program then sets values for several variables (3440). Min is set to 0 torr, Max is set to −300 torr, Delta is set to −20 torr, Duration is set to 1 second, and Timer is set to 0:00. The program sets the value for Pressure to Min (3445), and then applies force equal to Pressure in the lower chamber (3450).

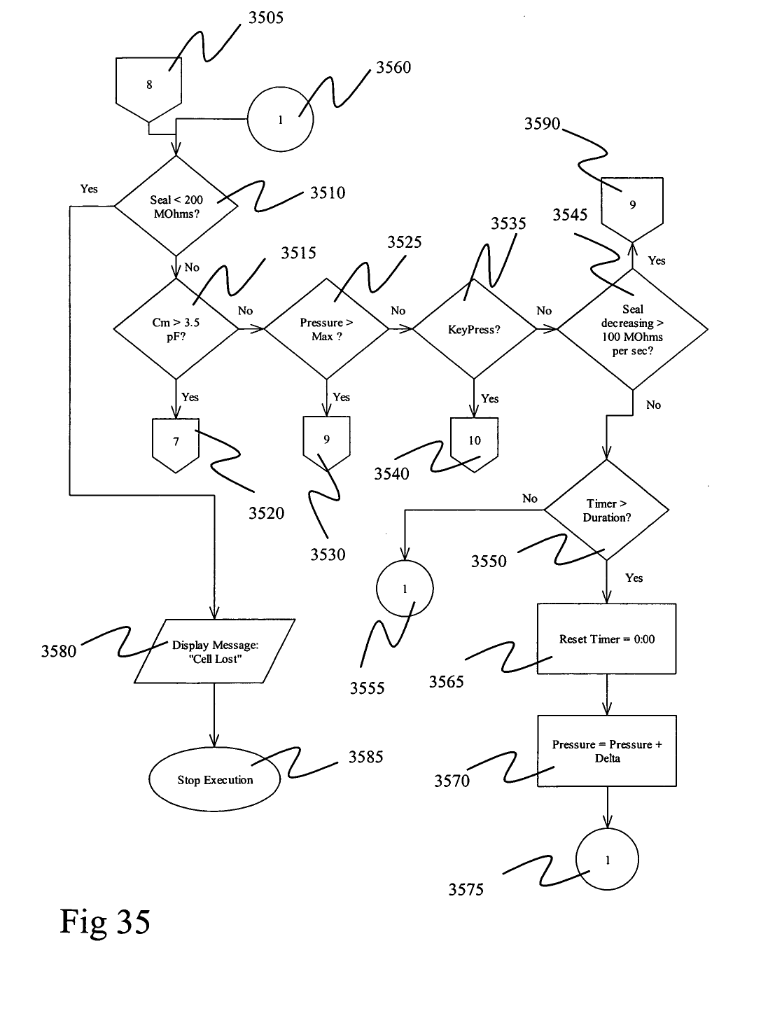

Procedure BreakIn continues at step 3510 as illustrated by FIG. 35, and as indicated by the illustrated off-page connector 3455 pointing to 3505. The program checks whether Seal is less than 200 megaOhms (3510). If yes, the program displays the message “Cell Lost” (3580), then stops execution of the program (3585). If at step 3510 the seal is not less than 200 megaOhms, the program checks if capacitance is greater than 3.5 pF (3515). If yes, Procedure BreakIn continues to step 3610 (as illustrated by off-page connector 3520 pointing to off-page connector 3605). If capacitance at step 3515 is not greater than 3.5 pF, the program checks whether Pressure is greater than Max (3525). If yes, Procedure BreakIn continues to step 3445 (as illustrated by off-page connector 3530 pointing to off-page connector 3460). If Pressure at step 3525 is not greater than Max, the program checks whether KeyPress has a value (3535). If yes, Procedure BreakIn continues to step 3710 (as illustrated by off-page connector 3540 pointing to off-page connector 3705). If no KeyPress value is found at step 3535, the program checks whether Seal is decreasing by greater than 200 megaOhms per second (3545). If yes, Procedure BreakIn continues to step 3445 (as illustrated by off-page connector 3590 pointing to off-page connector 3460). If at step 3545 Seal is not decreasing by greater than 200 megaOhms per second, the program checks whether Timer is greater than Duration (3550). If no, Procedure BreakIn goes to step 3510 (as illustrated by connector 3555 pointing to connector 3560). If at step 3550 Timer is greater than Duration, the program resets Timer to 0:00 (3565), then the program increments Pressure by Delta (3570). The Procedure then returns to step 3510 (as illustrated by connector 3575 pointing to connector 3560).

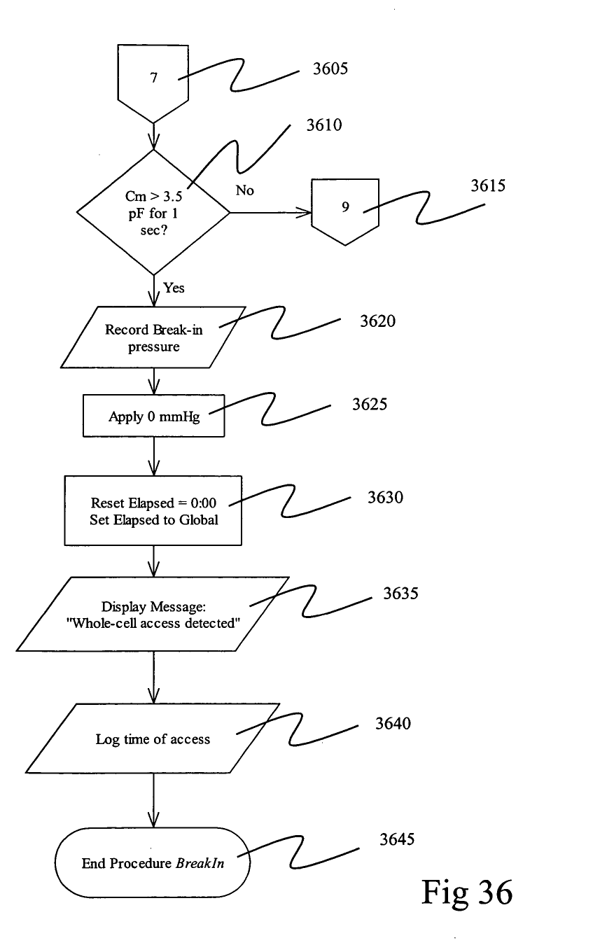

Procedure BreakIn continues as illustrated by FIG. 36. The program checks whether capacitance is greater than 3.5 pF for 1 second (3610). If no, Procedure BreakIn continues to step 3445 (as illustrated by off-page connector 3615 pointing to off-page connector 3460) to restart the pressure steps. If at step 3610, capacitance is greater than 3.5 pF for 1 second, the program records Break-in pressure to the log file (3620), and applies 0 torr of pressure (3625). The program then resets Elapsed to 0:00, then sets Elapsed to Global (3630). The whole cell access duration is set to the be a global variable. The program then displays the message “Whole-cell access detected” (3635), writes the time of access to the log (3640) and then Procedure BreakIn ends at step 3645. As illustrated by the program overview of FIG. 26, the program next continues to step 2625 and runs Procedure RaControl.

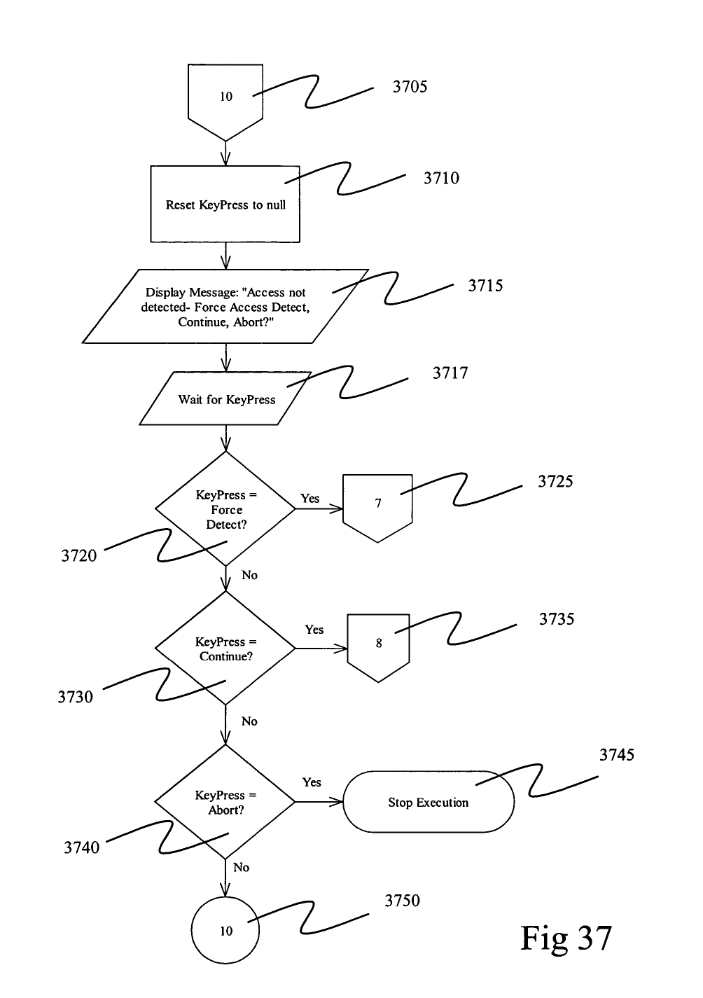

Procedure BreakIn continues as illustrated by FIG. 37. At step 3710, the program resets the value for KeyPress to null. Next, the program displays the message “Access not detected—Force access detect, Continue, Abort?” (3715) In step 3717, the program waits for the user to input whether to force access detect, continue or abort. The program checks for input by the user. If the users enters “Force access detect” (3720), Procedure BreakIn goes to step 3610 (as illustrated by off-page connector 3725 pointing to off-page connector 3605). If the user enters “Continue” (3730), Procedure BreakIn goes to step 3510 (illustrated by off-page connector pointing 3735 pointing to off-page connector 3505). If the user enters “Abort” (3740), the program stops executing (3745). If no input has been received by step 3740, the program returns to step 3705 and continues the input loop.

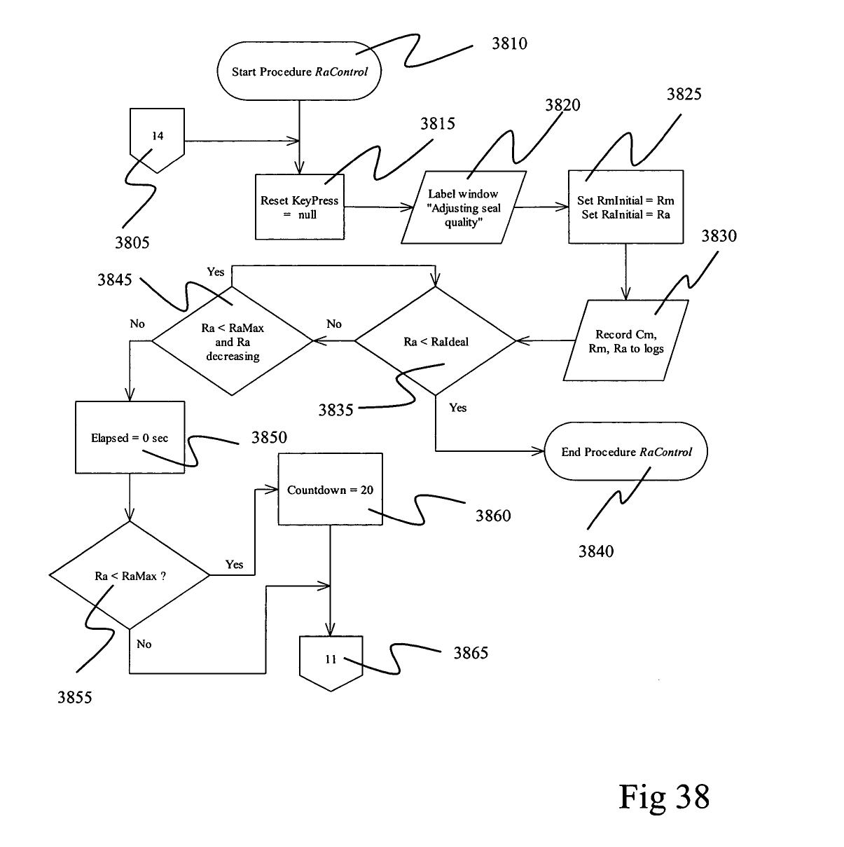

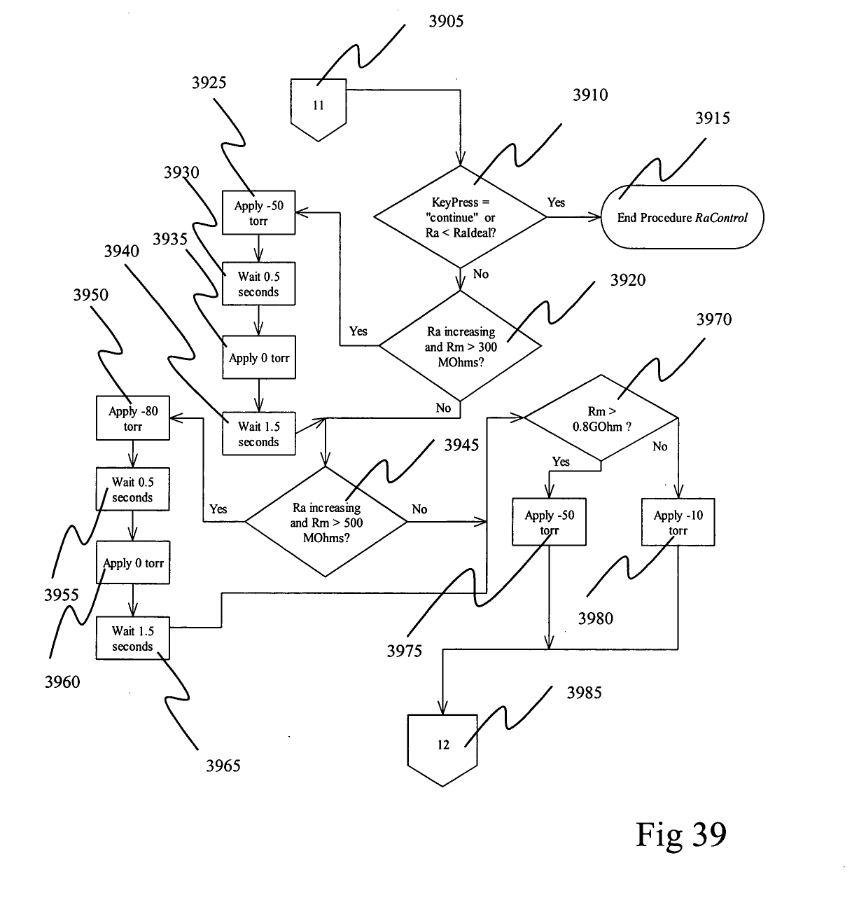

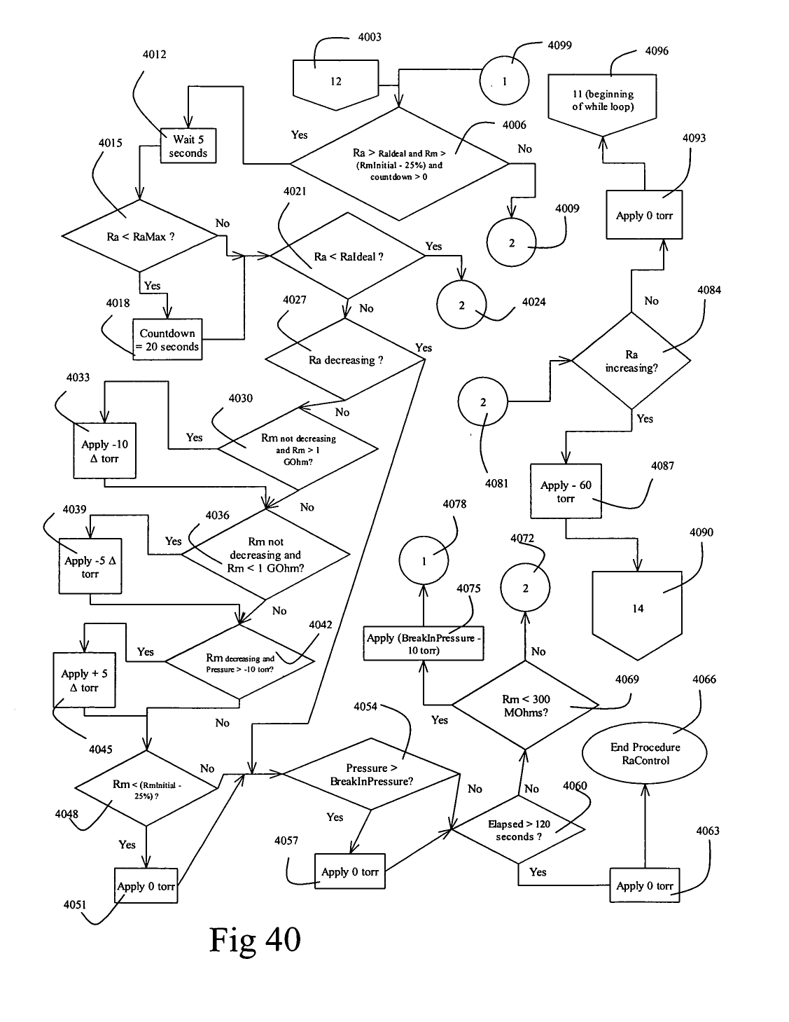

Procedure RaControl, as illustrated by FIGS. 38, 39, and 40, are now described. The program runs Procedure RaControl from step 2625. Procedure RaControl starts at step 3810. In step 3815, KeyPress is set to null. Next, the program displays the message “Adjusting seal quality” (3820). The program then assigns RmInitial the value of Rm, and assigns RaInitial the value of Ra (3825). The values for Cm, Rm, and Ra are recorded (3830). The program verifies if Ra is less than RaIdeal (3835). RaMax and RaIdeal are values that can be ascribed by the user beforehand. If yes, the procedure ends (3840). If Ra is not less than RaIdeal, then the program verifies if Ra is less than Ra Max and Ra is decreasing (3845). If yes, the program returns to step 3835. If the answer at 3845 is no, the program sets Elapsed to 0 seconds (3850), then the program verifies if Ra is less than RaMax (3855). If Ra is less than RaMax, then Countdown is set to 20 seconds (3860), and Procedure RaControl continues to step 3910 (as illustrated by off-page connector 3865 pointing to off-page connector 3905). If at step 3855 Ra is not less than RaMax, Procedure RaControl continues to step 3910 (as illustrated by off-page connector 3865 pointing to off-page connector 3905.

Procedure RaControl continues as illustrated by FIG. 39. At step 3910, the program checks whether the user has inputted “Continue” or whether Ra is less than RaIdeal. If yes, the procedure ends (3915). If the answer at step 3910 is no, the program goes to step 3920.

At step 3920, the program verifies if Ra is increasing and Rm is greater than 300 megaOhms. If no, the program continues to step 3945. If at step 3920 Ra is increasing and Rm is greater than 300 megaOhms, the program applies −50 torr of pressure (3925), waits 0.5 seconds (3930), applies 0 torr of pressure (3935), then waits 1.5 seconds (3940). The program then continues to step 3945. The program verifies if Ra is increasing and Rm is greater than 500 megaOhms (3945). If no, the program continues to step 3970. If at step 3945 Ra is increasing and Rm is greater than 500 megaOhms, the program applies −80 torr pressure (3950), waits 0.5 seconds (3955), applies 0 torr of pressure (3960), then waits 1.5 seconds (3965). The program then goes to step 3970.

At step 3970, the program checks if Rm is greater than 0.8 gigaOhm. If yes, it applies −50 torr of pressure (3975). If no, it applies −10 torr pressure (3980). From both steps 3975 and 3980, Procedure RaControl continues to step 4006 (as illustrated by off-page connector 3985 pointing to off-page connector 4003.

Procedure RaControl continues as illustrated by FIG. 40. The program checks, at step 4006, if Ra is greater than RaIdeal, if Rm is greater than (RmInitial−25%), and if countdown is greater than 0. If no, the program continues to step 4084 (as illustrated by connector 4009 pointing to connector 4081). If at step 4006 the answer is yes, then the program continues to step 4012 and waits 5 seconds. Then the program tests whether Ra is less than RaMax (4015). If yes, then the program sets Countdown to 20 seconds (4018), and will time down be seconds to zero and continues to step 4021. If at step 4015 Ra is not less than RaMax, the program continues to step 4021.

At step 4021, the program checks whether Ra is less than RaIdeal. If yes, the program continues to step 4084 (as illustrated by connector 4024 pointing to connector 4081). If at step 4021 Ra is not less than RaIdeal, the program checks whether Ra is decreasing (4027). If Ra is decreasing, the program continues to step 4054. If at step 4027 Ra is not decreasing, the program checks if Rm is not decreasing and Rm is greater than 1 gigaOhm (4030). If yes, −10 delta torr of pressure is applied (4033), and the program continues to step 4036. If at step 4030 the value is false, the program continues to step 4036. At step 4036, the program checks whether Rm is not decreasing and Rm is less than 1 gigaOhm. If yes, −5 delta torr of pressure is applied (4039) and the program continues to step 4042. If at step 4036 the answer is no, the program continues to step 4042. At step 4042 the program tests whether Rm is decreasing and Pressure is greater than −10 torr. If yes, +5 torr of pressure is applied (4045) and the program continues to step 4048. If at step 4042 the answer is no, the program continues to step 4048. At step 4048, the program checks whether Rm is less than (RmInitial−25%). If yes, 0 torr of pressure is applied (4051), and the program continues to step 4054. If at step 4048 the answer is no, the program continues to step 4054.

The program next checks whether Pressure is greater than BreakInPressure (4054). If yes, 0 torr of pressure is applied (4057), and the program continues to step 4060. If at step 4054 Pressure is not greater than BreakInPressure, the program continues to step 4060. The program checks whether Elapsed time is greater than 120 seconds (4060). If yes, 0 torr of pressure is applied (4063), and Procedure RaControl ends (4066). If at step 4060 Elapsed is not greater than 120 seconds, the program checks whether Rm is less than 300 megaOhms (4069). If no, the program continues to step 4084, as illustrated by connector 4072 pointing to connector 4081. If at step 4069 Rm is less than 300 megaOhms, pressure equal to (BreakInPressure less 10 torr) is applied (4075). The 5 program continues to step 4006, as illustrated by connector 4078 pointing to connector 4099.

At step 4084 the program checks whether Ra is increasing. If yes, −60 torr pressure is applied (4087) and the program continues to step 3815, as illustrated by off-page connector 4090 pointing to off-page connector 3805. If at step 4084 Ra is not increasing, 0 torr of pressure is applied (4093), and the program returns to the beginning of the loop at step 3910, as illustrated by off-page connector 4096 pointing to off-page connector 3905.

Once Procedure RaControl has ended, the program, in an unillustrated step, records and outputs the data, preferably to a database. These data can be recorded and outputted by a variety of means, including electronic storage media (hard disk or floppy disk), electronic transfer via a network (such as TCP/IP or Bluetooth), or optical storage media. Additionally, in an unillustrated step, the program may display the results on an output device, such as a LCD display or computer monitor screen. In another unillustrated step, the program may optionally generate a printout of the results and other collected data via a printing device such as a laser printer. The results gathered by the program may, in an unillustrated step, be collated, aggregated, or compared to other previous results, or control results. Depending upon the needs and requirements of the user of this present invention, the program can be configured to use one or more of the above-referenced output methods. Having completed these steps, and having outputted the results and/or data, the program stops execution (2630).

### EXAMPLES

### Example 1

### Device for Ion Transport Measurement Comprising Upper Chamber Piece and Biochip

An ion transport measuring device in the form of a cartridge known as the SEALCHIP™ (Aviva Biosciences, San Diego, Calif.) comprising an upper chamber piece and a chip comprising ion transport measuring holes was manufactured.

Upper chamber pieces with 16 wells having dimensions of 84.8 mm(long)×14 mm(wide)×7 mm(high) were injection molded with polycarbonate or modified polyphenylene oxide (NORYL®) material. The distance between centers of two adjacent wells was 4.5 mm. The well wall was slanted by 16 degrees on one side and 23 degrees and contoured on the other side to allow guidance for cell delivery. The well holes had a diameter of 2 mm.

A biochip with 16 laser-drilled recording apertures had dimensions of 82 mm (long)×4.3 mm (wide)×155 microns (thick). The distance between the first hole and a narrow edge is 7.25 mm. The holes were laser drilled to have two counterbores of 100 microns (diameter)×100 microns (deep) and 25 microns (diameter)×35 microns (deep), respectively. A final through-hole was drilled from the side of the counterbores and had a 7 to 9 micron entrance hole and a 2.0 micron exit hole with a total through-hole depth of 20 microns. Chemical treatment with acid and base was done as described in Example 3.

The treated chip was attached to the upper chamber using UV epoxy glue.

Devices produced using this methods had anRe of ˜2MOhm with standard ES and IS solutions, and an average Ra of ˜6.0MOhm using RBL cells with a standard pressure protocol described herein.

### Example 2

### A 52-Chip Bench Mark Study

We have conducted a bench mark study using 52 single-hole biochips tested using a CHO cell line expressing the Kv1.1 potassium channel. The result demonstrated a 75% success rate as determined by the following criteria: 1) achievement of sealing of at least one gigaohm (a “gigaseal”) within five minutes of cell landing on a hole, and 2) maintenance of Ra of less than 15MOhm, and Rm of greater than 200MOhm throughout 15 minutes of whole cell access time.

Chip Fabrication

Patch clamp chips were designed at Aviva Biosciences and fabricated using a laser-based technology (without an on-line laser measurement device). The K-type chips were made from ˜150 micron thick cover glass. The ion transport measuring hole structures had ˜140 micron double counterbores and final through-holes of ˜16.5±2 micron depth. The apertures on the recording surface had a diameter of 1.8±0.5 microns. The recording surface was further smoothed (polished) by laser.

Surface Treatment

Chips were received from FedEx overnight service and were inspected for integrity and cleanness. About 5% of the chips were excluded from further treatment in this process. Selected chips were then treated according to Example 3. Treated chips were stored in ddH₂O for 12 to 84 hours before the tests.

Batch QC for Chips

Chips were acid and base treated in batches of 20˜25. Four to six pieces of each batch were randomly picked for testing their patch clamp performance with CHO-Kv1.1 cells in terms of speed to seal and stability of the whole cell access. Batches with <75% success rate were excluded for the 50-chip tests.

Cell Passage

CHO-Kv1.1 cells (CHO cells expressing the Kv1.1 ion channel) between passage 47 and 54 were split daily at 1:10 or 1:15 for next-day experiments. Complete Iscove media (Gibco 21056-023) with 10% FCS, 1×P/S, 1×NEAA, 1×Gln, 1×HT with 0.5 mg/ml Geneticin was present in media used to passage cells and not present in media used to grow cells for next-day experiments.

Cell Preparation

Cells were isolated using the protocol for CHO cell preparation described in Example 6. After isolation, cells were resuspended in PBS complete media and passed through a 20 micron polyester filter into an ultra-low cluster plate (Costar 3473). The cells were used for the study between 30 minutes and 3 hour 30 minutes after the filtration.

Cell QC

Isolated cells were quality control tested with conventional pipette patch clamp recordings for their speed to seal, break-in pressure, and Rm and Ra stability. Freshly pulled pipettes were typically used within 3 hrs. Only cell preparations that passed the pipette quality control test were used for the 50-cell tests. About 50% of the preparations out of approximately 30 cell isolations passed and were used for this study.

Solutions

Intracellular solution was made according to the following formula: 8 mM NaCl; 20 mM KCl; 1 mM MgCl₂; 10 mM HEPES-Na; 110 mM K-Glt; 10 mM EGTA; 4 mM ATP-Mg; pH 7.25 (1M KOH3); 285 mOsm.

Aliquoted at 10 ml per 15 ml corning centrifuge tube, and stored at 4° C.

Extracellular solution (PBS complete) was DPBS (1×), with glucose, calcium and magnesium (Gibco cat #14287-080).

This solution contained:
0.9 mM CaCl₂, 2.67 mM KCl, 1.47 mM KH2PO4, 0.5 mM MgCl2, 138 mM NaCl, 8.1 mM Na2HPO4, 5.6 mM Glucose, 0.33 mM Na-pyruvate, pH 7.2-7.3, 295 mOsm.
   Chip Quality Control (QC)

For each recording, the chip was assembled into a two-piece cartridge, and the lower and upper chambers were filled with intracellular and extracellular solutions, respectively. The chip was further quality control tested by inspection under the microscope and seal-test resistance measurement. Chips that showed a dirty surface, visible cracks and/or had a seal test resistance greater than 2.1 MOhm were excluded.

Experiment Settings

Chips that passed quality control underwent electrode offset and the overall recordings were done with 4KHz bass filter. Cell landing was monitored on computer screen.

Criteria

A simple description of a positive result is: chips that achieved gigaseals and gave Ra<15MOhm and Rm>200MOhm throughout 15 min recording period.

Results

A total of 58 chips were tested, 6 of which were excluded from final analysis. Out of the 52 cells included, 39 (75%) passed the test criteria. 43 (83%) achieved at least 12 minutes of continuous high quality recordings (Ra<15MOhm; Rm>200MOhm); 47 (90%) achieved gigaseals.

Success Rate

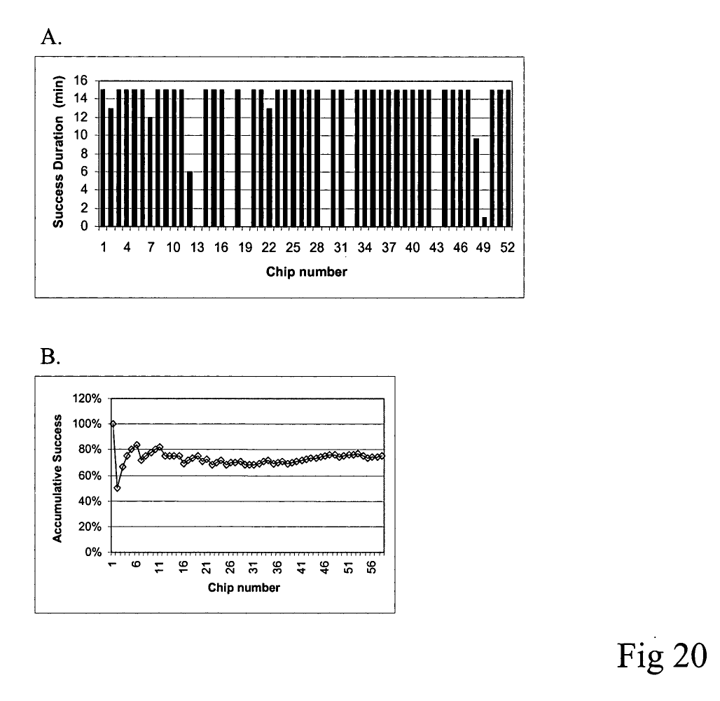

Success duration is plotted in FIG. 20A. Accumulative success rate is plotted in FIG. 20B. Success rate was consistent throughout the tests, which suggests that most of the critical experimental parameters were under control. 75% is a representative success rate under the current controlled conditions.

Electrode Resistance (Re)

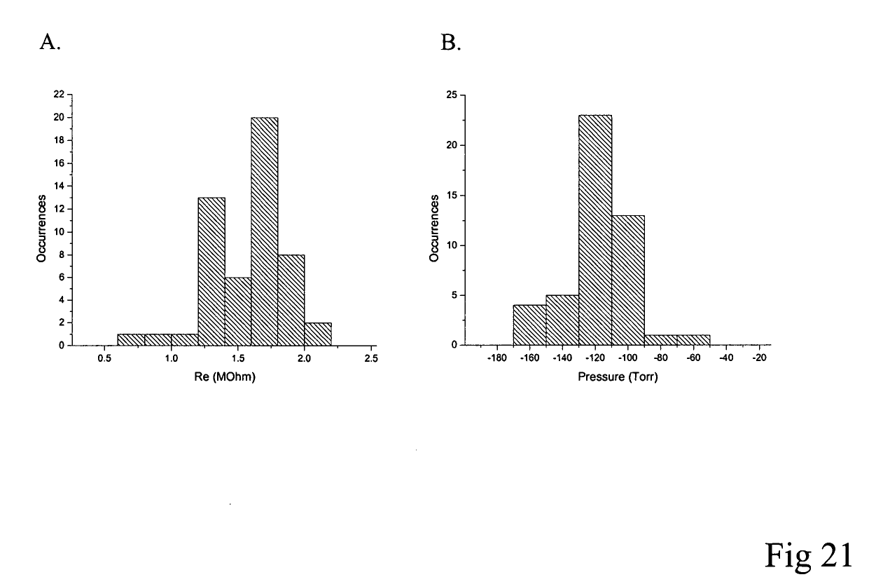

90% of the electrodes selected for the tests had Re between 1.3 to 2.0 MOhms (FIG. 21A). A total of 81 chips were mounted and tested. 23(28%) failed the quality control test, among which 15(18.5%) were due to Re>2.1 MOhms. 5(6%) chips were screened out because of their dirtiness of surface; 3(4%) had blocked or cracked holes. Chips were not screened at low Re values. The reason behind the 2.1 MOhm cut off is that historically chips with the current geometry (double counterbore) showed lower than 75% success rate in achieving the test criteria. Re is more or less normally distributed except for a slightly higher peak at ˜1.3MOhm.

Break-In Pressure

Break-in Pressure is an important parameter for cell condition. During the tests, break-in pressures were tightly distributed between −100 to −130 torrs (FIG. 21B). Our previous findings suggest that seals with more negative break-in pressure are likely to have higher and unstable Ra, while seals with lower break-in pressure are likely to have lower and unstable Rm.

Membrane Resistance (Rm)

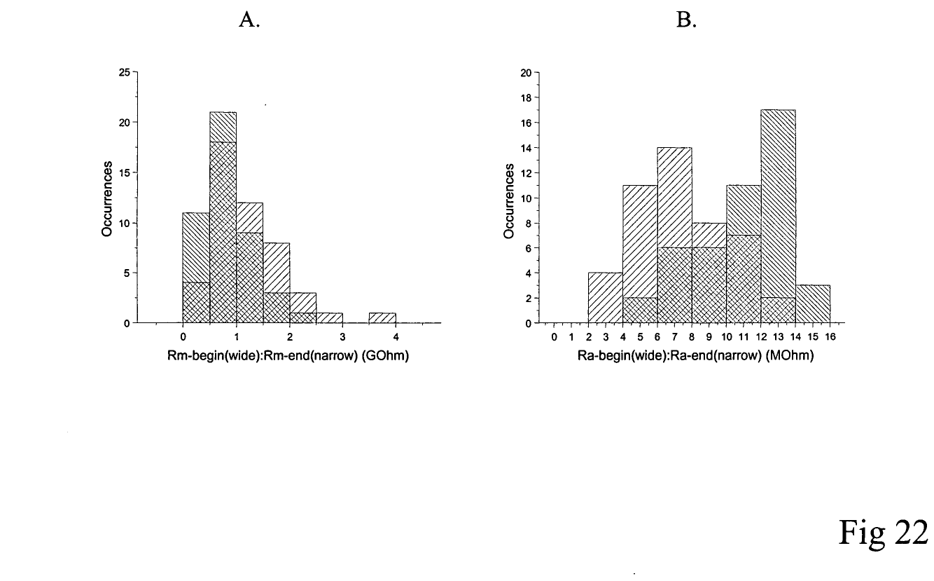

After break-in, Rm was mostly between 0.5 to 2MOhm (FIG. 22A). Ending Rm had a similar distribution, but more skewed to lower values. This is consistent with the deterioration of Rm over time. However, the amount of Rm deterioration was surprisingly small, which suggests that the seals were very stable during the 15 minutes test periods.

Access Resistance (Ra)

Initial Ra had a normal distribution centered at 7MOhm (FIG. 22B). 80% of the seals had Ra starting from below 10 MOhm. In most cases, Ra increased during the 15 minutes with an ending value near 11˜13MOhm. In order to minimize disruption of the seals, great effort was not made trying to maintain minimal possible Ra. It is not known what the ending Ra would be and what percentage of seals would lose Rm if such efforts were made.

Typical Recordings

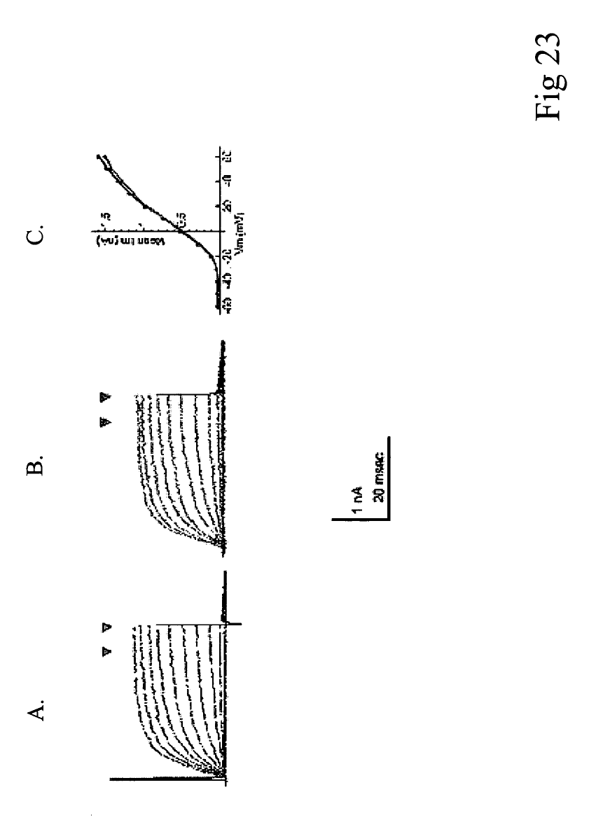

FIGS. 23-25 demonstrate sample data from one particular cell monitored during the 52-cell test referred to above. FIG. 23A demonstrates the whole-cell current record in response to a series of voltage steps from a holding potential of −80 mV to various potentials between −60 mV and +60 mV. FIG. 23B shows the potassium current, extracted from the whole-cell current by P/4 leak correction of the same currents, compensated for leak and capacitance. FIG. 23C illustrates the current-voltage relationship of the steady-state current averaged from data recorded at the time-points between the arrowhead indicators in FIG. 23A and FIG. 23B, showing the voltage-dependence of the potassium current expressed in this cell line. The larger currents were the uncompensated currents (from FIG. 23A) and the smaller currents were compensated (from FIG. 23B). The difference between the compensated and uncompensated currents represents the leak current, which was negligible in relation to total whole-cell current.

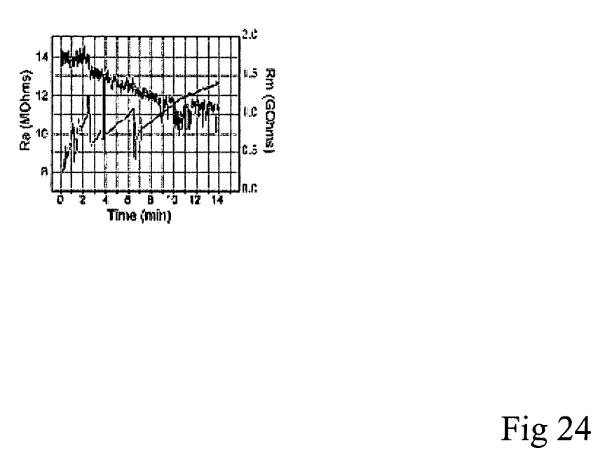

FIG. 24 shows data similar to those in FIG. 23 but is recorded at the end of a 15-minute recording period whereas data in was FIG. 23 recorded at the start of the recording period, where the duration of the recording period is relative to the time at which whole-cell access was achieved. FIG. 24A demonstrates the whole-cell current record in response to a series of voltage steps from a holding potential of −80 mV to various potentials between −60 mV and +60 mV. FIG. 24B shows the potassium current, extracted from the whole-cell current by P/4 leak correction of the same currents, compensated for leak and capacitance. FIG. 24C illustrates the current-voltage relationship of the steady-state current averaged from data recorded at the time-points between the arrowhead indicators in FIG. 24A and FIG. 24B, showing the voltage-dependence of the potassium current expressed in this cell line. Once again, in FIG. 24C, the leak current was still a small proportion of the whole-cell current.

FIG. 25 shows the time-course of the measured seal quality parameters during the same experiment that is represented in FIGS. 23 and 24. Over the 15 minute recording period, the membrane resistance (Rm) decreased (due to leak current) slightly from 1.4 GOhms to 1.0 GOhms, and access resistance (Ra) increased from 8 MOhms to 13 MOhms. The non-uniform time-profile of the traces is representative of the effect of the applied pressure control protocol used to control Ra during the experiment.

### Example 3

### Treatment of Ion Transport Measurement Chips to Enhance their Electrical Sealing Properties

Detailed Procedure: (referenced to step numbers below). All incubation processes were carried out in self-made Teflon or modified polyphenylene oxide (Noryl®) fixtures assembled in a glass tank while shaking (80 rpm, with C24 Incubator Shaker, Edison, N.J., USA). Water was always as fresh as practical from a water purification system (NANOpure Infinity UV/UF with Organic free cartridge). Nitric acid was ACS grade (EM Sciences NX0407-2, 69-70%). Sodium hydroxide was 10 N. meeting APHA requirements (VWR VWR3247-7). When necessary, chips were inspected for QC before and after treatment.

The protocol used was:
1. 3 hour shaking incubation in 6M nitric acid at 50 degrees C.
2. 6×2 minute rinses in DI water at room temperature.
3. 60 minute incubation in DI water (shaking)
4. 2 hour shaking incubation in 5M NaOH at 33 degrees C.
5. 6×2 minute rinses in DI water at room temperature.
6. 30 minute incubation in DI water (shaking) at 33 degrees C.
7. Chips were stored in DI water at room temperature. A vial used for storage was filled to the neck to minimize air space.

Chips treated according to this protocol demonstrated enhanced electrical sealing when tested in ion transport detection devices.

### Example 4

### Achieving Seals with Inverted Chips

A biochip was fabricated from Bellco D263 or Corning 211 glass of thickness of ˜155 micron. The 16 laser-drilled recording apertures on the chip had dimensions of 82 mm (long)×4.3 mm (wide)×155 microns (thick). The distance between the first hole and a narrow edge is 7.25 mm. The apertures were laser drilled to have one counterbore of 100 microns (diameter)×125 microns (deep). A final through-hole was drilled from the side of the counterbores and had a ˜10 micron entrance hole and 4.5 micron exit hole with a total through-hole depth of 30 microns. After standard chemical treatment as described in Example 3, the biochip was mounted to an upper chamber piece described in Example 1 in inverted configuration such that the counterbore side faced the upper chamber piece (where RBL cells were added). Recordings were done with a device adapted to Nikon microscope as described in Example 5. Typical voltage clamp quality parameters such as Rm and Ra over time are shown in FIG. 22.

### Example 5

### A Biochip Device Adapted to a Microscope and Having Flow-Through Lower Chambers

A device for ion transport measurement known as the “Tester” device having flow-through lower chambers was designed and constructed. The device has a lower chamber base piece that formed the bottom surfaces of the lower chambers and comprises conduits for the inflow and outflow of solutions, and a gasket that formed the walls of the lower chambers. The device also comprises a cartridge that provided upper chambers and a chip comprising holes. The device was adapted for a microscope, so that the bottom surfaces of the lower chambers are transparent, and the device was fitted to a baseplate adapted to a microscope stage. The following description of the design and manufacture of the device makes reference to FIGS. 3-8.

In this design, a biochip cartridge that has a chemically-treated glass chip sealed to an upper chamber piece can be assembled onto a microscope stage-mounted lower chamber base piece that allows simultaneous or sequential testing of all recording apertures while simultaneously observing the experiment's progression microscopically.

The Tester device includes a metallic base plate, in this case made of aluminum, shaped to insert onto a microscope stage, and sculpted to support and align a multi-well perfusion lower chamber base piece. The baseplate of the device (as shown in FIG. 4) was shaped to take advantage of an existing mounting point on the Nikon microscopes by positioning the device into an aperture within the microscope stage. It is round, with an edge intended to prevent it from falling through the hole on the stage. The depth of the device is intended to hold the functional portion of the biochips as well as the cells that are added to the biochip at testing time at a convenient focal point within the focal range of the microscopes, that is, at essentially the same level as the upper platform of the microscope stage.

To assemble the device, a gasket (as shown in FIG. 6) was inserted over the lower chamber base piece (301 in FIG. 3A) seated in a baseplate, then the cartridge, was clamped onto the gasket by compression via a clamp assembly (shown in FIGS. 7A and 7B) that bolted onto the base plate using four thumb-screws (73 in FIG. 7A). The lower chamber piece was made of plastic and contained an array of 16 conduits for inflow of intracellular solution, and another 16 conduits for outflow of same. The 32 conduits emerged on the top surface of the lower chamber base piece in alignment with the recording apertures of the biochip. The gasket was made of PDMS and was situated between the lower chamber piece and the chip, and contained slits, or holes (601 in FIG. 6), that aligned between the emerging holes of the perfusion conduits of the lower chamber piece and the recording apertures of the chip, such than intracellular “lower” chambers were formed within the array of slits or holes in the gasket. An electrode of silver-silver chloride was introduced into each of the 16 outflow conduits along one side of the base piece to function as recording electrodes.

With reference to FIG. 8A, the device was made up of 1) a metallic base plate (812), specifically, but not exclusively, stainless steel, 2) a transparent lower chamber piece (801), sometimes referred to as an “inner chamber array”, made from polycarbonate (but could be any other convenient transparent substance) 3) electrodes (not visible in Figure) inserted into the outflow conduits of the lower chamber piece, made from wires of silver or any other conductor capable of being used as a voltage sensing and current-delivering electrode, and attached to a connector on the outer side of the lower chamber piece, 4) inert tubing connectors (not visible in FIG. 8; 302 as seen in FIG. 3A) glued to the lower chamber base piece at the conduit openings (or any other means that may provide a connection for a fluid conveyance system) in this case made from glass, 5) a gasket (805) that provided a seal between the lower chamber base piece and the biochip cartridge, where the gasket (in this case made of PDMS) simultaneously comprised the inner chambers, 6) a biochip cartridge (804) mounted onto the test apparatus over the gasket, and held in place by a guidance system, in this case alignment pins inserted into the plastic bottom chamber array body in such a way as to restrict movement of the biochip while simultaneously guaranteeing alignment of the biochip's recording surface with the inner chambers, 7) a clamp (802) assembly intended to apply sufficient pressure onto the biochip cartridge so as to generate a seal between the bottom of the chip and the gasket, and 8) an array of electrodes (not visible in FIG. 8, 75 in FIG. 7B)attached to the clamp shaped and oriented so as to enter into the top wells of the biochip cartridge, all 16 at a time, and where all electrodes were connected together so as to provide a reference electrode in the upper chambers of the cartridge.

FIG. 5 shows the arrangement of parts installed in the baseplate (54) schematically. The clamp (53) holds the cartridge (51) down on the gasket (not visible) and lower chamber base piece (not visible). The clamp has attached electrode wires (55) that extend into the upper wells of the cartridge (51). This depiction also shows the lower chamber electrode array (52) of pin sockets (56) that connect to electrode wires that are threaded through conduits leading to lower chambers. The pin sockets (56) can be connected to the signal amplifier.

FIG. 8B showed the assembled device, in which the clamp (802) is screwed into the baseplate (812). The flow-through lower chamber base piece is not visible beneath the cartridge (804). Inflow tubing (809) is attached to one side of the lower chamber base piece and outflow tubing (808) is attached to the opposite side of the lower chamber base piece.

1) Metallic Base Plate:

This base plate serves multiple functions. First, the metallic body serves as an electrical noise shield for the bottom side of the test chamber. It completes a type of faraday cage that is contiguous with the grounded stage of the microscope. Secondly, the metal base was carved on the top side so as to catch any fluids that may leak or spill and prevent the contamination of the microscope with said fluids. To this end, the base plate was sealed, with silicone glue or with silicone grease (vacuum grease) or with any other such viscous immiscible substance (eg: Vaseline) to the transparent lower chamber piece described in 2) (below). Third, the base plate was shaped to optimize its use with a particular microscope. Specifically, in our case it was desirable for the base plate to be cut to fit onto the 107 mm circular cutout hole of a Nikon microscope. Fourth, the base plate was drilled and tapped so as to provide a mounting point for the lower chamber piece and for the clamp of the Tester. Its design was such that held the recording aperture of the cartridge within a few millimeters of the level of the top of the microscope stage so as to ensure that the chip function could be monitored within the focal range of the microscope. FIG. 4 illustrates the design of the base plate as adapted for the Nikon Microscope.

2) Transparent Lower Chamber Base Piece (Inner Chamber Array):

This design of a lower chamber base piece, shown as (301) in FIG. 3A may also be referred to as an inner chamber array, or an intracellular chamber array. For the convenience of being able to view under a microscope the progression of an experiment, it was made of a transparent material. Polycarbonate was chosen for its ease of machining. Its shape was designed to support a cartridge over it, and provide tubing connections along the long edges of either side the cartridge, as well as to provide connections to electrodes placed inside one of each pair of conduits (holes in the base piece material that function as such) supplying each recording aperture of the chip. The conduits drilled into each side provided a connection between the edge of the lower chamber base piece and somewhere near the center, then another conduit was drilled perpendicularly from the top surface to connect to each conduit coming from the edge. The emerging conduits at the top surface were located so as to provide for an inflow and an outflow of solution to and from each of the lower chambers. The lower chamber base piece did not comprise chambers, but instead the lower chambers were created by openings within the gasket material. As seen in FIG. 3B, the inflow and outflow conduit openings (304) in the areas (303) of the upper surface of the base piece that corresponded to the bottom surfaces of the lower chambers were separated from one another so as to leave an undisturbed area of surface that could be seen through with a microscope so as to visualize the recording aperture during experimentation. To this end, the top surface that was in opposition to the chip was untouched with the exception of the emerging inflow and outflow conduit openings and as well the bottom surface of the lower chamber base piece was left untouched so as to not disrupt transparency of the part. Each conduit leading to the edges of the base piece had a means (such as tubing connectors) for interfacing it to inflow tubing and outflow tubing (309 and 308 in FIG. 3B) (see also description of part 4) that provided for delivery of solutions, as well as for pneumatic pressure control. Tubing connectors (302) can be seen in FIG. 3A. One of the conduits going to the edge of the part was left longer so as to house an electrode (wire) that is introduced into the lumen of the conduit. The added length also allowed for a second segment to be glued onto the top surface so as to house the connectors for the electrodes. The top surface of this part was trimmed down around the periphery of area covered by the cartridge so as to provide an edge that functioned to hold the gasket in place during mounting and removal of the cartridge. Further, between each pair of inflow and outflow holes for each bottom well was a cut intended to prevent wetting of the gasket material to span from one bottom chamber to adjacent bottom chambers. This lower chamber base piece as a whole contained 6 pin holes 2 mm in diameter to hold 6 pins that functioned to keep the cartridge aligned during mounting. It also contained a further 4 holes to hold 4 spring-pins (307 of FIG. 3B) that functioned to provide an electrical connection for an early version of the cartridge. The present version of the cartridge does not require these contacts, however they were kept in place so as to prevent contact with the gasket before the clamp part is pressed down during the mounting. Finally, two more holes were present so as to use two screws to hold the part onto the base plate.

3) Inner Chamber Electrodes:

Each lower chamber contained an electrode, which in this case is a silver wire that was periodically chlorided. The wire was inserted into the lumen of the longer conduit of the base piece and bent upward into the electrode connector array (315 in FIG. 3B). The segment of wire was sufficiently long that it remained exposed within the lumen of the longer conduit after the inert tubing interface parts were glued into place, and the other end was soldered to a connector, in this case an array of 1 mm female pin-connector sockets inserted into holes in the part. The connector pin sockets (310) are seen in FIG. 3B.

4) Inert Tubing Interface:

Into each conduit of the base piece an inert tubing connector (in this case made from glass) was inserted that was fixed in place with epoxy glue. Epoxy was chosen only in so much as it is preferred for bonding glass to polycarbonate. The tubing segments were sufficiently long to butt against a countersunken segment of the conduit drilled into the lower chamber piece and stick out of the part enough to hold a segment of silicone tubing that was press-fit onto the glass segment. This junction should withstand a pressure greater than two atmospheres positive pressure, and greater than 700 mmHg vacuum pressure. It was determined that 3 to 5 mm insertion into the silicone tubing was sufficient to accomplish this requirement.

5) Gasket:

For convenience the flexible gasket was molded from curing PDMS. The gasket contained a raised edge on the bottom side that surrounded the chambers as a whole and was able to hug an edge present in the same periphery on the lower chamber piece so as to hold the gasket in place. As depicted in FIG. 6, the gasket had oblong holes (601) in it that aligned over the exit and entrance holes of the lower chamber piece for each chamber of the array. On the top surface of the gasket was a set of squared O-rings (602) that were part of the gasket but raised sufficiently to form a seal onto the cartridge when pressed against it with the clamp part.

5) Biochip

The fabrication of chips having holes for ion transport measurement has been described herein. In this device, the chip was made of glass and has 16 laser drilled holes. The chip was laser polished on the top surface, and treated in acid and base prior to attaching the chip in inverted orientation to an upper chamber piece with a UV adhesive.

6) Clamp Assembly:

A clamp was made from an inflexible material so as to not allow bowing of the cartridge during compression onto the gasket while mounted on the tester. In this case it was made of stainless steel for its inertness when wetted with physiological buffers. The clamp was shaped so as to fit snugly over the cartridge and was drilled so as to accommodate and be positioned by the guide-pins sticking out of the lower chamber piece. Four screws were finger-tightened to the base plate at each corner of the clamp assembly so as to press down the cartridge to seal it against the gasket. This part is shown in FIG. 7A and 7B.

7) Upper Chamber Electrodes:

In early development it was expected that compression pins would contact the bottom of the cartridge during testing to provide a connection to the reference electrodes built in to the cartridge. The present embodiment of the cartridge does not contain reference electrodes, therefore these electrodes were introduced into the top wells of the cartridge. To this end, periodically chlorided silver wires were used as electrodes. The electrodes were shaped to dip deep inside each well, and on the outside of the wells the wires were soldered to a wire running along the top of the clamp part (visible in FIG. 7B). At each end of this wire was a 1 mm female pin connector that was used to interface with the voltage clamp amplifier. The upper chamber electrode wires (55) are shown in FIG. 5.

Method:

Before use the device should be clean and dry.

A SealChip™ cartridge was removed from its carrier, and rinsed with a jet of deionized water of approximately 18 MOhms resistance. The product was them dried under a stream of pressurized dry air filtered through a 0.2 μm air filter to remove water from the recording apertures and their vicinity.

The clean cartridge was then placed with top-wells upward onto the pressure contact pins of the tester such that movement of the cartridge was limited by the six alignment dowels of the bottom chamber piece. Prior to clamping the cartridge to the gasket and lower chamber base piece, the cartridge should be supported above the gasket but without yet touching the gasket. The clamp was them placed over the cartridge such that the four mounting holes aligned with their threaded counterparts on the base plate. The four mounting screws were them used to press down the clamp uniformly thereby pressing the cartridge down onto the PDMS gasket with sufficient pressure to form a tight seal between the chip and the gasket and between the gasket and the lower chamber base piece. The recording aperture within each chamber of the cartridge should already be aligned with openings in the gasket that form the lower chambers.

The bottom chambers were then filled from one side with sufficient solution (analogous to intracellular solution) to fill the bottom chambers and fill enough of the tubing on the other side such that capacitative distension of the tubing on the filling side would not introduce air into the recording chamber, and would not introduce air into the area of the tubing that contained the bottom-chamber electrode. (For this purpose, it is best to fill the chamber starting from the side that does not contain the electrode since higher pressures will be used for vacuum pressure than for positive pressure, thereby ensuring that the electrode will remain in full contact with the solution at all times.) Once the bottom chamber was filled and was free of visible bubbles, the tubing was sealed off by a clamp (a valve or any means that ensures electrical isolation between the bottom chambers of the array can also be used). Sufficient positive pressure was applied to the free end of the inner chamber tubing so as to cause solution to be forced into the counterbore and through the hole of the recording aperture of the chip.

Once solution was seen emerging into the top chamber, the pressure was released, and immediately the top chamber was filled with sufficient solution (analogous to extracellular solution) so as to completely immerse the top side of the chip without bubbles remaining on the chip surface, and to fill the top well sufficiently to provide good contact with the electrode in the top well. (It is also of benefit to fill the top well sufficiently to avoid a strong meniscus effect (60 to 70 microliters with the present version of the SealChip™ product) whenever it is intended to view under an inverted microscope the progression of the experiment (for upright microscopes it is necessary to fill with more solution, ˜90 microliters, to allow good contact with a coverslip that must be placed over the well to enable a good view of the bottom of the well).)

The assembled tester, now ready for testing, was placed on the microscope (and connected to the voltage clamp amplifier(s) as well as to the pressure control device(s) for testing.

After the termination of the experiment, the tester was disconnected and removed from its testing location. The extracellular medium was suctioned from each well, and each well was rinsed once with deionized water to removed any leftover particulate (debris or cellular) material that may have been left over from the experiment. Both ends of the tubing of the bottom chambers were then opened and the solution was suctioned out of the bottom wells. Each well was well rinsed with clean deionized water, then dried completely with pressurized air. Finally the screws holding down the clamp were removed and the cartridge was disassembled from the tester. Any wetting at the gaskets was wicked away with a lint-free tissue. (If any liquid is pooled around the gasket, then the gasket should be removed, rinsed then dried, and the bottom chamber array should be likewise rinsed and dried, ensuring that the tubing is also rinsed and completely dried.)

Quality Control/Quality Assurance of SealChip™ product:

Internally to the company, the “tester unit” device described in this example has been used for QC/QA of the SealChip™ product before it is sent to a customer, and before it is used internally for further research. The success rate with a product that passes the QC has been as good as that with older testers that tested a single chamber at a time.

Quality Control/Quality Assurance of Cells:

Internally to the company, the tester unit device has been used to verify the quality of the cells used for QC/QA using known good SealChip™ product.

Research and Development:

The tester unit has been used by our company for testing variations to the SOP for the SealChip™ product. In the future it may be used for discovery and screening of compounds that require exchanging of solutions on the bottom well or where compounds or particles must be delivered to the cytosolic chamber after a seal is formed with the cell membrane.

A great number of results have been achieved on the microscope adapted device (“Tester Unit”) since its development. The tester unit has been the tool of choice for performing quality control experiments on the SealChip™ product. The following gives examples of the quality of data obtained from it. (The seal resistance is designated Rm; G refers to GigaOhms and M refers to MegaOhms.)

TABLE 1
SealChip ™ Data

| Chip Lot# | Hole ID | Cell Type | Re(G) | Rm(G) | Ra(M) | Seal Qlty | Note |
|---|---|---|---|---|---|---|---|
| S2N22-40 | C | RBL | 3.4 | 0.5 | 5.7 | +++ | |
| | G | | 3.3 | 5 | 6.7 | +++ | |
| | I | | 3.3 | 2 | 2 | +++ | |
| | M | | 3.2 | 0.25 | 8.8 | +++ | |
| | O | | 3.2 | 0.5 | 6.5 | +++ | |
| S2D18-114 | A | | 3.9 | 2.4 | 7.2 | ++ | |
| | C | | 3.9 | 2.2 | 18 | + | |
| | G | | 3.7 | 4 | 10.8 | ++ | |
| S2D20-28 | B | | 4.5 | 2.6 | 9.1 | ++ | |
| | C | | 4.2 | 1 | 13.3 | −S | |
| | D | | 4.4 | 0.6 | 10.5 | + | |
| | E | | 4.3 | 2.7 | 10 | ++ | |
| | F | | 4.3 | 1.6 | 10 | ++ | |
| | G | | 4.2 | 3.5 | 9.4 | ++ | |
| | H | | 4.3 | 3.3 | 8.8 | ++ | |
| S2D20-8 | A | | 4.1 | 1.7 | 12.2 | ++ | |
| | C | | 4.1 | 2.7 | 9.3 | ++ | |
| | G | | 4.2 | 1.7 | 8.4 | ++ | |
| | I | | 4.1 | 2 | 11 | + | |
| | M | | 4.1 | 1.6 | 11.7 | S | Debris landed before cell |
| | O | | 4.1 | 2.6 | 7.6 | ++ | |
| S2D20-50 | A | | 4.3 | 2.9 | 12.4 | + | |
| | B | | 4.3 | 7 | 10.7 | +++ | |
| | C | | 4.1 | 1.1 | 10 | +++ | |
| | D | | 4.3 | 2.1 | 8.8 | +++ | |
| | E | | 4.2 | 4.5 | 8 | ++ | |
| | F | | 4.4 | 4.9 | 7.1 | ++ | |
| | G | | 4.3 | 1.5 | 10 | ++ | |
| | H | | 4.3 | 6.9 | 8.3 | ++ | |
| | I | | 4.2 | 6.2 | 8.3 | +++ | |
| | J | | 4.2 | 0.6 | 8.1 | +++ | |
| | K | | 4.3 | 0.9 | 9.8 | ++ | |
| | L | | 4.4 | 6.5 | 7.4 | +++ | |
| | N | | 4 | 6 | 7.7 | +++ | |
| | O | | 4 | 5.6 | 7.8 | ++ | |
| | P | | 4.1 | 6.5 | 12.8 | +++ | |
| S2D219-21 | D | | 3.1 | 4.5 | 4.6 | +++ | |
| | E | | 3 | 1.5 | 11.6 | + | |
| | F | | 3 | 1.5 | 5.6 | ++ | |
| | G | | 3 | 2.8 | 5.8 | +++ | |
| | H | | 3 | 3.1 | 4.8 | +++ | |
| | I | | 3 | 3.2 | 8 | ++ | |
| | J | | 3.1 | 3 | 5.7 | ++ | |
| S2D18-191 | A | | 3.5 | 3.3 | 8.5 | ++ | |
| | C | | 3.5 | 2 | 13.9 | ++ | |
| | D | | 3.3 | 1.6 | 8.9 | ++ | |
| | E | | 3.6 | 2.5 | 9.2 | +++ | |
| | F | | 3.6 | 2 | 8 | +++ | |
| | G | | 3.5 | 0.4 | 7.7 | +++ | |
| | H | | 3.7 | 1.4 | 6 | ++ | |
| S2D18-206 | A | | 3.3 | 4.1 | 7 | +++ | |
| | C | | 3.2 | 2.1 | 6.2 | ++ | |
| | D | | 3.3 | 4.6 | 6.7 | ++ | |
| | E | | 3.4 | 3.4 | 5.2 | ++ | |
| | F | | 3.1 | 0.7 | 5.8 | +++ | |
| | H | | 3.4 | 0.6 | 11 | S | |
| S2D20-6 | B | | 4.1 | 1.5 | 8.8 | ++ | |
| | C | | 4.3 | 0.5 | 8.9 | ++ | |
| | D | | 4.1 | 3.2 | 8.9 | +++ | |
| | E | | 4.1 | 3.3 | 6.8 | +++ | |
| | G | | 4.3 | 3.8 | 7.8 | +++ | |
| | H | | 4.3 | 3.2 | 10.4 | +++ | |
| S2D20-133 | A | | 4.3 | 3.5 | 9.6 | S | |
| | B | | 4.5 | 4.4 | 7.5 | ++ | |
| | C | | 4.4 | 5 | 11.4 | ++ | |
| | D | | 4.5 | 3.1 | 10.8 | + | |
| | E | | 4.5 | 5.3 | 10 | +++ | |
| | F | | 4.4 | 5.1 | 8.8 | ++ | |
| | G | | 4.4 | 5.1 | 8.5 | ++ | |
| | H | | 4.3 | 1.1 | 10.5 | + | |
| S2D21-70 | A | | 4.2 | 2.1 | 22 | + | Spec near the hole |
| | B | | 4.2 | 2.7 | 8 | +++ | |
| | C | | 4.3 | 2.8 | 7.6 | +++ | |
| | D | | 4.3 | 1.3 | 12.3 | ++ | |
| | E | | 4 | 2.3 | 10.2 | ++ | |
| | F | | 4.2 | 0.5 | 7.2 | +++ | |
| S2D20-130 | A | | 3.2 | 0.8 | 7.6 | + | |
| | B | | 3 | 0.5 | 8.9 | ++ | |
| | E | | 3 | 1.3 | 11.1 | ++ | |
| | F | | 3.3 | 2 | 7.9 | +++ | |
| | G | | 3.3 | 0.5 | 11.9 | S | |
| | H | | 3.1 | 2.1 | 7.8 | ++ | |
| S2D20-194 | A | | 3.7 | 2.3 | 9.8 | +++ | |
| | C | | 3.6 | 3 | 7.9 | ++ | |
| | D | | 3.8 | 2.4 | 14 | S | |
| | E | | 3.6 | 2.4 | 5.9 | ++ | |
| | F | | 3.9 | 2.1 | 12.1 | ++ | |
| | G | | 3.7 | 2.1 | 6.7 | +++ | |
| | H | | 3.8 | 0.9 | 8.3 | ++ | |
| S2D18-81 | A | | 3 | 1.6 | 5.5 | ++ | |
| | C | | 3.1 | 2.1 | 6 | ++ | |
| | D | | 3.3 | 3.4 | 7.8 | ++ | |
| | E | | 3.3 | 2 | 6.6 | ++ | |
| | F | | 3.3 | 2.4 | 8.6 | ++ | |
| | G | | 3.4 | 2.9 | 8.6 | + | |
| | H | | 3.3 | 2.8 | 5.7 | +++ | |
| S2D20-171 | C | | 3.8 | 2.3 | 9.5 | ++ | |
| | E | | 3.8 | 2.7 | 8.3 | ++ | |
| | F | | 3.9 | 3.4 | 8.1 | ++ | |
| | G | | 3.7 | 3.3 | 6.2 | +++ | |
| | H | | 3.7 | 2.8 | 7.8 | +++ | |
| | I | | 3.7 | 2.8 | 12.7 | + | |
| | J | | 3.8 | 3.3 | 5.9 | +++ | |
| S2D16-26 | A | | 3.3 | 1.5 | 5.5 | ++ | |
| | C | | 3.5 | 1.9 | 7.5 | +++ | |
| | D | | 3.7 | 1.2 | 6.8 | ++ | |
| | E | | 3.5 | 1.7 | 7.5 | +++ | |
| | F | | 3.7 | 1.7 | 6.4 | +++ | |
| | H | | 3.7 | 1.7 | 8.8 | ++ | |
| S2D19-20 | A | | 2.5 | 1.4 | 5.7 | ++ | |
| | C | | 2.5 | 1.8 | 4.5 | +++ | |
| | D | | 2.5 | 1.5 | 5.8 | ++ | |
| | E | | 2.5 | 1.1 | 5 | ++ | |
| | F | | 2.4 | 1.8 | 1.6 | +++ | |
| | G | | 2.7 | 1.4 | 4.8 | ++ | |
| | H | | 2.8 | 1.6 | 5 | ++ | |
| S2D16-1 | B | | 3.2 | 1.2 | 10.3 | S | |
| | C | | 3.1 | 1.6 | 6.5 | + | |
| | D | | 3.1 | 0.6 | 17 | S | |
| | E | | 2.9 | 2.3 | 6.1 | ++ | |
| | F | | 3.1 | 2.7 | 6.1 | ++ | |
| | G | | 3.1 | 2.7 | 7.8 | +++ | |
| S3210-181 | A | Cho-Herg | 4.6 | 0.3 | 14 | +++ | |
| | B | | 4 | 0.5 | 11 | +++ | |
| | D | | 4 | 0.2 | 14 | ++ | |
| | E | | 4 | 1.3 | 17 | ++ | |
| | G | | 4 | 2.1 | 10 | +++ | |
| | H | | 4.1 | 0.6 | 12 | S | |
| S3214-60 | A | | 3.6 | 1.2 | 7 | ++ | |
| | B | | 2.9 | 1 | 7 | +++ | |
| | C | | 2.9 | 0.4 | 17 | −S | |
| | D | | 2.9 | 1.3 | 11 | + | |
| | G | | 3.1 | 1.7 | 10 | +++ | |
| | H | | 3 | 0.2 | 10 | ++ | |
| 031103-A1 | B | RBL | 3 | 1 | 4 | ++ | |
| | D | | 3.4 | 0.5 | 5.2 | ++ | |
| | F | | 3.1 | 1.1 | 4.1 | +++ | |
| | H | | 3 | 1.2 | 7 | ++ | |
| | N | | 3.2 | 0.4 | 4.4 | ++ | |
| | P | | 3.1 | 0.3 | 5.5 | + | |
| 031103-A2 | A | | 3.8 | 0.6 | 4.1 | ++ | |
| | C | | 4.3 | 2.1 | 4.1 | ++ | |
| | I | | 4 | 2.3 | 8.1 | ++ | |
| | K | | 4.4 | 2.1 | 5.3 | +++ | |
| | M | | 4.8 | 2.3 | 7.8 | ++ | |
| | O | | 4.4 | 2.7 | 9.9 | ++ | |
| 030703-A1 | A | | 3.6 | 1.9 | 4.9 | ++ | |
| | C | | 3.7 | 2.3 | 3.6 | +++ | |
| | E | | 3.8 | 2.2 | 5.8 | ++ | |
| | G | | 3.7 | 1.8 | 5.2 | ++ | |
| | I | | 3.4 | 1.7 | 4.1 | ++ | |
| | M | | 3.5 | 2.1 | 5.4 | ++ | |
| | O | | 3.7 | 1.7 | 4.6 | ++ | |
| 031103-A3 | A | | 4.8 | 2.5 | 5.2 | ++ | |
| | C | | 4.6 | 1.4 | 5.4 | ++ | |
| | E | | 4.8 | 1 | 4.6 | ++ | |
| | I | | 4.9 | 0.3 | 6.1 | ++ | |
| | D | | 4.9 | 1.6 | 6.1 | ++ | |
| | F | | 4.9 | 0.7 | 8.1 | ++ | |
| 030603-A2 | B | | 4.3 | 1.2 | 4.2 | +++ | |
| | C | | 4.3 | 4 | 9.2 | ++ | |
| | F | | 4.3 | 2 | 8.2 | ++ | |
| | H | | 4.4 | 2.2 | 7 | ++ | |
| | G | | 4.6 | 2 | 7.7 | +++ | |
| | I | | 4.2 | 2.2 | 7.2 | +++ | |
| | J | | 4.3 | 1.2 | 5.8 | ++ | |
| 030603-A1 | A | | 4.4 | 1.8 | 6.4 | + | |
| | B | | 4.4 | 1.2 | 8 | ++ | |
| | C | | 4.6 | 1.2 | 8 | + | |
| | F | | 4.8 | 1.5 | 8.5 | + | |
| | G | | 4.4 | 0.7 | 4.4 | ++ | |
| | H | | 4.3 | 1.4 | 5.9 | + | |
| | I | | 4.2 | 1.1 | 8.6 | ++ | |
| 030603-A3 | B | | 4 | 1.7 | 6.9 | +++ | |
| | D | | 4 | 0.28 | 6.9 | ++ | |
| | F | | 4.2 | 0.35 | 4.4 | ++ | |
| | H | | 4.3 | 0.27 | 6.9 | ++ | |
| | L | | 4.4 | 0.25 | 7.2 | ++ | |
| | N | | 4.5 | 0.85 | 7.2 | ++ | |

### Example 6

### Cell Preparation for Ion Transport Measurement

Part I. CHO wt. and CHO.Kv Cells

1. Use cells @ 50%˜70% confluency. (18 hrs after cells seeded 1:10˜1:15)

2. Remove medium and wash ×2 with X⁺⁺-free PBS (extra wash might be necessary if the final cell suspension has too much small debris)

3. Treat for 2′15″ with 1:10 trypsin-EDTA, at this time the supernatant might be a little turbid due to release of cells into the buffer.

4. Rock gently, aspirate to discard supernatant. Wait for 1′25″.

5. Add 1 volume of X⁺⁺-free DMEM complete with 10% FCS, NEAA, etc, rock gently to loosen and detach cells, and spin down (do not try to blow to remove the remaining cells sticking to the bottom)

6. Wash ×1 with PBS complete

7. Resuspend in PBS, triturate, and pass through 15˜20μm filter into non-stick plate.

Cells can be used after 10 minutes of recovery and should last for up to 4 hr

Part II. Transiently Transfected CHO Cells.

1. Remove medium and wash ×2 with X⁺⁺-free PBS

2. Treat for 1′ with 5 ml 1:10 trypsin-EDTA (0.5 ml 0.05% trysin 0.53 mM EDTA from GIBCO cat. No.25300-54 in 4.5 ml PBS)

3. Rock gently, aspirate to discard supernatant.

4. Add 0.5 ml fresh 1:1 trypsin-EDTA, Wait for 6 mins.

5. Add 5 ml of X⁺⁺-free DMEM complete with 10% FCS, NEAA, etc, rock gently to loosen and detach cells, leave cell at RT for 1 hour, and spin down (do not try to blow to remove the remaining cells sticking to the bottom)

6. Wash ×2 with 1 ml PBS complete

7. Resuspend in PBS, triturate, and pass through 15 to 20 micron filter into non-stick plate.

Part III. CHO-Herg Cells.

1. Use cells at 50%˜70% confluency in T-25 flasks (VWR, Cat. No. 29185-302).

2. Remove medium and wash ×2 with X⁺⁺-free PBS (extra wash might be necessary if the final cell suspension has too much small debris)

3. Treat for 1′ with 2 ml trypsin-EDTA(0.5 ml 0.05% trysin 0.53 mM EDTA from GIBCO cat. No.25300-54 in 1.5 ml PBS)

4. Rock gently, aspirate to discard supernatant. Wait for 2 mins.

5. Add 5 ml volume of X⁺⁺-free DMEM complete with 10% FCS, NEAA, etc, rock gently to loosen and detach cells, leave cell at RT for 30 min, and spin down (do not try to blow to remove the remaining cells sticking to the bottom)

6. Wash ×2 with 1 ml PBS complete

7. Resuspend in PBS, triturate, and pass through 15˜20 micron polyester filter into non-stick plate if cells still cluster together.

Part IV. Protocol for Isolation of CHO

1. Use cells at 70˜80% confluences in T-25 flasks (24 hrs after seeding).

2. Remove medium and wash ×2 with X⁺⁺-free PBS ((cell should not be leave in X⁺⁺-free PBS more than 10 mins, otherwise, the minimal digestion time will be decreased)

3. Wash once with 1:4 AccuMax (available from Innovative Cell Technologies, San Diego, Calif.) (wait about 20 second, rocking to removed the loose attached cell)

4. Treat at 37° C. w 4 ml volume of 1:4 Accumax (diluted with X⁺⁺-free PBS) for minimal time (cell dissociate from the flask and floated in the Accumax ) or 1.5 times minimal time.

CHO-KV

a. 1:4 AccuMax 5′ (1 ml AccuMax +3 ml X⁺⁺-free PBS) w/o rocking

b. 1:4 AccuMax 8′ (1 ml AccuMax +3 ml X⁺⁺-free PBS) w/o rocking

CHO-HERG

c. 1:4 AccuMax 8′ (1 ml AccuMax +3 ml X⁺⁺-free PBS) w/o rocking

d. 1:4 AccuMax 12′ (1 ml AccuMax +3 ml X⁺⁺-free PBS) w/o rocking

5. Add 5 ml volume of Ca⁺⁺-free DMEM with 10% FBS, into the flasks, and removed all cell suspension to a 15 ml centrifuge tube, spin down ˜300g×3 min (do not try to blow to remove the remaining cells sticking to the bottom).

6. Discard supernatant, add 1 ml 1:4 (PBSC:PBS), gently triturate to resuspend cell, centrifuge 2000 rpm×1 min in an micro centrifuge tube.

7. Discard the supernatant, add 800 μl to 1 ml 1:4 (PBSC*:PBS), triturate, and pass through 15˜20 micron filter into non-stick plate.

Part V. Protocol for Isolation of HEK

1. Use HEK-Na cells at 70˜80% confluences in T-75 flasks (16 hrs after seeding).

2. Remove medium and wash ×2 with X⁺⁺-free PBS

3. Add 6 ml X⁺⁺-free PBS, incubate at 37° C. for 5 mins, aspirate supernatant

4. Add 6 ml X⁺⁺-free PBS, incubate at 37° C. for 10 mins or until all cells dissociate from flask.

5. Add 2 ml Accumax directly into flask to finalize the Accumax concentration to 1:4, incubate cell at 37° C. for 4 mins

6. Add 6 ml volume of Ca⁺⁺-free DMEM with 10% FCS into the flasks to stop the digestion

7. Put cell mixture into a 15 ml tube, and spin down 300 g×3 min

8. Discard supernatant, gently suspend cell in 4 ml Ca⁺⁺ free DMEM with 10% FCS, incubate cell at 37° C. incubator at least 30 mins or until use it.

9. Carefully remove the supernatant, wash ×1 with PBS with 100 nM Cacl₂, 1 mM Mgcl₂

10. Triturate, resuspend cell in PBS with 100 nM Cacl₂, 1 mM Mgcl₂, filter cell mixture through 21 μm filter into non-stick plate.

### Example 7

### Program Logic and Pressure Control Profile

The following is a typical program logic for software pneumatic control. It includes procedures for cell landing, form seal, break-in, and Ra control.

```
#start of program
Count=0
Turn off compensations
Procedure Landing:
  Reset button_pressed
  Label window “Attempting Landing”
  Run washer # deliver clean ES to top chamber
  Wait 5 seconds
  Stop washer
  Repeat twice:
    Apply −300torr pressure # clear holes of any remaining debris after filling
    Wait 0.5 seconds
    Apply 0torr pressure
    Wait 2 seconds
  End repeat
  Zero junction potential
  Wait for stable reading
  Record average Re value
  Save Re to logs
  Initiate cell addition
  Wait until 0.5 seconds before cell delivery # before pipette touches ES
  Apply +10torr # before and during delivery
  Wait for pipette removal # from ES chamber
  Apply 0 torr
  Wait 3 seconds
  Apply −50torr
  Wait until Seal > 2Re for 0.5sec or elapsed=15 seconds
  If elapsed then
    Count=count+1
    If count >= 3 then abort test and write to log
    Apply +50torr
    Run proc Landing
  Endif
  Run FormSeal
End procedure
Reset elapsed
Procedure FormSeal
  Reset button_pressed
  Label window “Attempting Seal”
  Apply −80mV HP #negative holding immediately after landing
  Apply −50torr #this may not necessarily be the same as that used for landing
  While Seal increasing >20MOhms/second
    Wait until Seal >= 1Gohm or elapsed=10 seconds
  Endwhile
  Apply 0torr
  Wait 2 seconds
  While seal increasing >20MOhms/second and seal<1GOhm,
    Wait 1 second
  Endwhile
  #start ramping to attempt seal
  Unless seal>1GOhm, Apply ramp from 0torr to −50torr over 20 seconds
  Unless seal>1GOhm, Wait 5 seconds
  Unless seal>1GOhm, Apply 0torr
  Unless seal>1GOhm, wait 5 seconds
  Unless seal>1GOhm, Apply ramp from −30torr to −80torr over 30 seconds
  Unless seal>1GOhm, Wait 5 seconds
  Unless seal>1GOhm, Apply 0torr
  Unless seal>1GOhm, wait 5 seconds
  Unless seal>1GOhm, Apply ramp from −50torr to −100torr over 40 seconds
  Unless seal>1GOhm, Wait 5 seconds
  Unless seal>1GOhm, Apply 0torr
  Unless seal>1GOhm, wait 5 seconds
  Unless seal>1GOhm, Apply ramp from 0torr to −200torr over 120 seconds
  Unless seal>1GOhm, Wait 5 seconds
  Unless seal>1GOhm, Apply 0torr
  Unless seal>1GOhm, wait 5 seconds
  If not seal>1GOhm
      Check button_pressed
      If button_pressed = “continue” then abort test and write to log
      Run FormSeal
  Endif
  #Seal detected, now check stability
  Stop ramping and hold last pressure
  Wait 1 second # let seal stabilize
  If seal>1GOhm,
    Apply 0torr
    Record Seal value into Rseal, save to logs
    Unless Seal<(Rseal−200MOhms) or Seal decreasing >200MOhms/second
      Wait 5 seconds
    End unless
    If Seal<(Rseal−200MOhms) or Seal decreasing >200MOhms/second
      Check button_pressed
      If button_pressed = “continue”, goto Procedure BreakIn
      Run FormSeal
    Endif
    #cell sealed
  Endif
End Procedure
Procedure BreakIn:
  Reset button_pressed
  Label window “Attempting break-in”
  Null chamber capacitance
  Until capacitance > 3.5pF or Pressure>300torr or Seal<(Rseal−200MOhms) or Seal
decreasing >200MOhms/second
    Wait 1 second
    Apply −20 delta torr
  End until
  If capacitance > 3.5pF
    Record break-in pressure value
    Wait 0.5 seconds
    Apply 0torr
    Run procedure RaControl
  Endif
  If Pressure>300torr
    Apply 0torr
    Until capacitance > 3.5pF or Pressure>300torr or Seal<(Rseal−200MOhms) or
  Seal decreasing >200MOhms/second
      Wait 1 second
      Apply −20 delta torr
      Apply Zap
    End until
    If pressure>300torr then abort test and write to log
  Endif
  If capacitance > 3.5pF
    Record break-in pressure value
    Wait 0.5 seconds
    Apply 0torr
    Run procedure RaControl
  Endif
  If Seal<(Rseal−200MOhms) or Seal decreasing >200MOhms/second
    Check button_pressed
    If button_pressed = “continue”, goto Procedure BreakIn
    Run FormSeal
  Endif
End Procedure
Elapsed = 0
Procedure RaControl:
  Reset button_pressed
  Label window “Adjusting seal quality”
  Record Cm, Rm, Ra to logs
  Assign RmInitial = Rm, RaInitial = Ra
  If Ra < RaIdeal then end #RaIdeal does not need adjustment
  If Ra < RaMax and Ra decreasing then end #no need for adjustment
  If Ra < RaMax then countdown = 20 seconds else countdown = “true”
  While countdown
    Check button_pressed
    If button_pressed = “continue” then end
    If Ra increasing and Rm > 300MOhms
      Apply −50torr
      Wait 0.5seconds # max 2 seconds
      Apply 0torr
      Wait 1.5 seconds
    Endif
    If Ra increasing and Rm > 500MOhms
      Apply −80torr
      Wait 0.5seconds # max 2 seconds
      Apply 0torr
      Wait 1.5 seconds
    Endif
    If Rm>0.8GOhm then apply −50torr else apply −10torr
    While Ra>RaIdeal and Rm>(RmInitial−25%) and countdown
      Unless Ra<RaIdeal or Rm<(RmInitial−25%), wait 5 seconds
      If Ra<RaMax then countdown=20 seconds
      If Ra<RaIdeal then Endwhile
      If Ra not decreasing
        If Rm not decreasing and Rm>1GOhm then Apply −10 delta torr
        If Rm not decreasing and Rm<1GOhm then Apply −5 delta torr
        If Rm decreasing and Pressure>10torr then Apply +5 delta torr
        If Rm<(RmInitial−25%) then apply 0 torr
      Endif
      if pressure>BreakInPressure then apply 0torr
      If elapsed > 120 seconds then apply 0torr and end
      If Rm<300MOhms then apply (reakInPressure−10torr)
    Endwhile
    If −10torr>pressure>−50torr
      Apply 0torr
      If Ra increasing then apply −60torr
      If Ra increasing then run RaControl Procedure
    Endif
  Endwhile
End Procedure
```

### Example 8

### Achieving High Resistance Seals in 52-Cell Test

An operator using a syringe based pressure system employed a pressure control profile similar to that described in Example 7, except that it was performed manually rather than by computer automation. The 52-cell test described in Example 2 was performed using a syringe controlled by had while the operator viewed a pressure monitor.

The criteria for the test was the achievement of at least 75% success rate, with success defined as achieving a gigaohm seal to initiate a patch clamp, then during the patch clamp membrane maintaining resistance above 200 MOhms and maintaining access resistance (or series resistance) below 15 MOhms for at least 15 minutes. Table 2 demonstrates the conclusion from this experiment, showing that the goals of the 52-cell test were met.

FIGS. 23-25 give a sample of the time-course of an experiment where membrane resistance and access resistance values are kept within the acceptable parameters. At many locations in the recording there are deflections in the access resistance trace (FIG. 25). These deflections represent locations where the pressure protocol was applied to maintain the seal quality parameters. The success rate at achieving gigaohm seals is demonstrated in FIG. 20. This data is a graphical representation of the data identified in Table 2, where 90% of the chips produced a gigaohm seal with CHO cells. FIG. 22 shows a histogram of the parameters achievable with this pressure control protocol. Data shown with wide diagonal bars represents initial values for Ra and Rm, and values with narrow diagonal bars represent values for Ra and Rm after 15 minutes of continuous whole-cell access under voltage clamp conditions. These data demonstrated that overall, 75% of the cells achieved gigaohm seals, and then whole-cell access was attained with acceptable parameters that were well-controlled for at least 15 minutes.

TABLE 2
50-cell test that demonstrates the feasibility of the
pressure control protocol.
Success Rate Data

| | No. of Chips | Proportion |
|---|---|---|
| Total chips tested | 52 | 100% |
| Chips achieved gigaseals | 47 | 90% |
| Chips achieved >12′ | 43 | 83% |
| continuous recordings | | |
| Chips achieved >15′ | 39 | 75% |
| continuous recordings | | |

### Example 9

### Single Channel Recording Using a Biochip Comprising a Hole for Ion Transport Measurement

RBL cells were prepared for patch clamp recording by simple centrifugation. The cells were then delivered onto an ion transport measurement device with a single recording aperture. The biochip device was assembled according to Example 2. The biochip had been treated with acid and base to improve sealability. The upper chamber solution was PBS lacking calcium and magnesium. The lower chamber solution was: 150 mM KCl, 10 mM HEPES-K, 1 mM EGTA-Na, 1 mM ATP-Mg pH (KOH) 7.4, the upper chamber solution was:
8 mM NaCl, 20 mM KCl, 1 mM MgCl₂, 10 mM HEPES-Na, 125 mM K-Glu, 10 mM EGTA-K, 1 mM ATP-Mg pH (KOH) 7.2.

Seal formation was achieved as provided in the previous examples, but after gigaseal formation, no break-in step was performed. Single-channel recordings were obtained from a cell-attached membrane patch on an RBL cell. An inward rectifier IRK1 single channel was recorded in RBL cells. A low concentration of extracellular K⁺ which does not depolarize the cell and does not inactivate the channel was used. ATP was present in the internal solution, which prevents the rundown of the channel activity. The noise level of the recordings was reduced from 10 pA to 1 pA in order to observe single channel events, which have an amplitude of a few picoamps.

The devices and methods described herein can be combined to make additional embodiments which are also encompassed in the present invention.

All headings are for the convenience of the reader and should not be used to limit the meaning of the text that follows the heading, unless so specified.

All references cited herein, including patents, patent applications, and publications are incorporated by reference in their entireties.

1-164. (canceled)

165 A device for ion transport measurement, comprising:
an upper chamber piece that comprises at least one well, wherein said at least one well is open at its upper and lower ends; and
a chip that comprises at least one ion transport measuring means, wherein said chip has been treated to enhance the electrical sealing properties of said at least one ion transport measuring means;
wherein said chip is attached to the bottom of said upper chamber piece such that each of said at least one ion transport measuring means is in register with one of said at least one well.

166 The device of claim 165, wherein said chip has been treated to make said at least one ion transport measuring means more electronegative.

167 The device of claim 166, wherein at least a portion of said chip has been treated with at least one base.

168 The device of claim 165, wherein said at least one ion transport measuring means is at least one hole through said chip.

169 The device of claim 165, wherein said chip comprises glass, silicon, silicon dioxide, quartz, one or more plastics, one or more polymers, one or more waxes, one or more ceramics, polydimethylsiloxane (PDMS), or a combination thereof.

170 The device of claim 168, wherein said chip is able to form a seal with a cell or particle, wherein said seal has a resistance (R) of greater than 200 megaOhms.

171 The device of claim 170, wherein said chip is able to form a seal with a cell or particle, wherein said seal has a resistance (R) of greater than 500 MegaOhms.

172 The device of claim 171, wherein electrical access between said chip an the inside of said cell or particle, or between said chip and the outside of said cell or particle in the region of said hole has an access resistance that is less than the seal resistance (R).

173 The device of claim 172, wherein access resistance between said chip and said particle is less than 80 MegaOhms.

174 The device of claim 172, wherein access resistance between said chip and said particle is less than 30 MegaOhms.

175 The device of claim 172, wherein access resistance between said chip and said particle is less than 10 MegaOhms.

176 The device of claim 165, wherein said chip is attached to the bottom of said upper chamber piece in inverted orientation.

177 The device of claim 165, wherein said upper chamber piece comprises one or more plastics, one or more polymers, one or more ceramics, one or more waxes, silicon, or glass.

178 The device of claim 165, wherein said at least one well has an upper diameter of from about 0.05 millimeter to about 20 millimeters.

179 The device of claim 178, wherein said at least one well has a depth of from about 0.01 millimeter to about 25 millimeters.

180 The device of claim 165, wherein said at least one well tapers downward at an angle of from about 0.1 degree to about 89 degrees from vertical.

181 The device of claim 165, wherein said upper chamber piece comprises at least one electrode.

182 The device of claim 181, wherein said upper chamber piece comprises one electrode, further wherein said one electrode contacts each of said at least one well.

183 The device of claim 181, wherein said upper chamber piece comprises at least two wells and at least two electrodes, wherein each of said at least two electrodes contacts one of said at least two wells.

184 The device of claim 168, wherein said chip is attached to said upper chamber piece with one or more adhesives.

185 The device of claim 168, wherein said chip is attached to said upper chamber piece by pressure mounting.

186 The device of claim 168, further comprising a lower chamber piece attached to the bottom side of said chip that can form at least a portion of at least one lower chamber.

187 The device of claim 185, wherein said lower chamber piece comprises at least one gasket.

188 The device of claim 186, wherein said at least one lower chamber is a flow-through lower chamber.

189 The device of claim 188, wherein said device further comprises a lower chamber base piece comprising at least one inflow conduit and at least one outflow conduit.

190 The ion transport measuring device of claim 189, wherein said at least one well is at least two wells and said at least one ion transport measuring means is at least two ion transport measuring means.

191 The device of claim 190, comprising at least one lower chamber.

192 The device of claim 191, wherein each of said at least one lower chamber accesses one of said at least one well via said hole in said biochip.

193 The device of claim 192, wherein said device comprises two or more lower chambers, wherein at least two of said lower chambers access one of said at least two upper chambers via a hole in said biochip.

194 The device of claim 192, wherein each of said at least two wells comprises, contacts, or is in electrical communication with at least one electrode, further wherein each of said at least one lower chambers comprises, contacts, or is in electrical communication with at least one electrode.

195 A method of measuring at least one ion transport activity or property, comprising:
i) filling at least one lower chamber of the device of claim 194 with a measuring solution;
ii) adding a one or more cells or particles to one or more of at least one well of the device, wherein each of the one or more of the at least one well is connected to one of the at least one lower chambers that comprises measuring solution via a hole in the ion transport measuring chip;
iv) applying pressure to said at least one lower chamber or at least one well to create a high-resistance electrical seal between at least one cell or particle and said at least one hole; and
v) measuring at least one ion transport property or activity of the at least one cell.

196 The method of claim 195, wherein said at least one cell or at least one particle is at least one cell.

197 The method of claim 195, wherein said applying pressure to said at least one lower chamber or at least one well can be under automated control.
